(19) United States
(12) Patent Application Publication
(10) Pub. No.: US 2026/0176322 A1
(43) Pub. Date: Jun. 25, 2026

(54) COMPOUNDS

(71) Applicant: IP2IPO Innovations Limited, London (GB)

(72) Inventor: Stephen Robert BLOOM, London (GB)

(21) Appl. No.: 19/551,950

(22) Filed: Feb. 27, 2026

Related U.S. Application Data
(63) Continuation of application No. 18/266,571.

(30) Foreign Application Priority Data
Dec. 11, 2020 (GB) 2019588.9
Jul. 27, 2021 (GB) 2110809.7

(51) Int. Cl. C07K 14/605; A61K 38/26; A61P 3/04

(52) U.S. Cl. CPC C07K 14/605; A61K 38/26; A61P 3/04

(57) ABSTRACT

The disclosure provides novel compounds which are peptide hormone analogues, and which are useful in treating disorders such as diabetes and obesity. The compounds of the general sequence recited in the specification possess a tailored profile with regards to potency properties at the glucagon and GLP-1 receptors. With regard to in vivo properties, administration of example peptides of the invention have been shown, in animal models, to result in increased weight loss. Preferred compounds achieve this without reducing food intake significantly.

### CROSS-REFERENCE TO RELATED APPLICATIONS

The present application is a continuation of U.S. patent application Ser. No. 18/266,571, filed Jun. 9, 2023, which is the National Stage of International Application No. PCT/GB2021/053249, filed Dec. 10, 2021, which claims the benefit of and priority to Great Britain Patent Application No. 2019588.9, filed on Dec. 11, 2020, and Great Britain Patent Application No. 2110809.7, filed on Jul. 27, 2021, the contents of each of which are incorporated herein by reference in their entireties.

### REFERENCE TO THE ELECTRONIC SEQUENCE FILE

The contents of the electronic sequence listing (METS_019_04US_SeqList_ST26.xml; Size: 1,130,221 bytes; and Date of Creation: Feb. 25, 2026) are herein incorporated by reference in their entirety.

### FIELD OF THE INVENTION

The present disclosure relates to compounds which are peptide hormone analogues, and which are useful in treating disorders such as diabetes and obesity.

### BACKGROUND

According to the National Health and Nutrition Examination Survey (NHANES, 2009-2010), 33.0% of adults in the United States aged 20 and over are overweight, 35.7% are obese, and 6.3% are extremely obese. In addition, a large percentage of children in the United States are overweight or obese.

The cause of obesity is complex and multi-factorial. Increasing evidence suggests that obesity is not a simple problem of self-control but is a complex disorder involving appetite regulation and energy metabolism. In addition, obesity is associated with a variety of conditions associated with increased morbidity and mortality in a population. Although the etiology of obesity is not definitively established, genetic, metabolic, biochemical, cultural and psychosocial factors are believed to contribute. In general, obesity has been described as a condition in which excess body fat puts an individual at a health risk.

There is strong evidence that obesity is associated with increased morbidity and mortality. Disease risk, such as cardiovascular disease risk and type-2 diabetes disease risk, increases independently with increased body mass index (BMI). Indeed, this risk has been quantified as a five percent increase in the risk of cardiac disease for females, and a seven percent increase in the risk of cardiac disease for males, for each point of a BMI greater than 24.9 (see Kenchaiah et al., N. Engl. J. Med. 347:305, 2002; Massie, N. Engl. J. Med. 347:358, 2002).

Diabetes is a chronic syndrome of impaired carbohydrate, protein, and fat metabolism owing to insufficient secretion of insulin or to target tissue insulin resistance. It occurs in two major forms: insulin-dependent diabetes mellitus (type 1 diabetes) and non-insulin dependent diabetes mellitus (type 2 diabetes). Diabetes type 1, or insulin dependent diabetes mellitus (IDDM) is caused by the destruction of p cells, which results in insufficient levels of endogenous insulin. Diabetes type 2, or non-insulin dependent diabetes, results from a defect in both the body's sensitivity to insulin, and a relative deficiency in insulin production. According to the National Diabetes Statistics Report, 2014 around 28.9 million adults in the United States aged 20 and over have diabetes (2009-2012 National Health and Nutrition Examination Survey estimates applied to 2012 U.S. Census data). In adults 90 to 95% of the diabetes is type 2 diabetes.

There is substantial evidence that weight loss in obese persons reduces important disease risk factors. Even a small weight loss, such as 10% of the initial body weight in both overweight and obese adults has been associated with a decrease in risk factors such as hypertension, hyperlipidemia, and hyperglycemia. It has been shown that considerable weight loss can effectively cure type 2 diabetes (Lim et al., Diabetologia June 2011).

Although diet and exercise provide a simple process to decrease weight gain, overweight and obese individuals often cannot sufficiently control these factors to effectively lose weight. Pharmacotherapy is available; several weight loss drugs have been approved by the Food and Drug Administration that can be used as part of a comprehensive weight loss program. However, many of these drugs have serious adverse side effects. When less invasive methods have failed, and the patient is at high risk for obesity related morbidity or mortality, weight loss surgery is an option in carefully selected patients with clinically severe obesity. However, these treatments are high-risk, and suitable for use in only a limited number of patients. It is not only obese subjects who wish to lose weight. People with weight within the recommended range, for example, in the upper part of the recommended range, may wish to reduce their weight, to bring it closer to the ideal weight. Thus, a need remains for agents that can be used to effect weight loss in overweight and obese subjects as well as in subjects who are of normal weight.

A number of approaches to the development of agents useful in effecting weight loss have involved gastrointestinal peptide hormones and their analogues. For example, derivatives of peptides deriving from the preproglucagon molecule have been proposed for use in treatment of obesity and/or diabetes. Preproglucagon is a precursor peptide of glucagon-like peptide 1 (GLP-1), as well as other hormones including glucagon GLP-1 is produced in vivo in the intestinal L cell in response to the presence of nutrients in the lumen of the gut. GLP-1 possesses a number of physiological functions including increasing insulin secretion from the pancreas in a glucose-dependent manner, decreasing glucagon secretion from the pancreas, inhibiting gastric emptying and decreasing food intake by increasing satiety. Increased insulin secretion leads to a decrease in circulating glucose concentration.

Examples of research into analogues of such peptides are described in, for example, WO2013/004983 which describes peptide molecules containing sequence from both the GLP-1 and glucagon peptides. WO2015/132599 also discloses peptide hormone analogues, which are derivable from preproglucagon and which are useful in the therapy of disorders such as obesity and diabetes. WO2017/178829 discloses compounds that are analogues of exendin-4, GLP-1 and oxyntomodulin which have a modified ligand bias and therapeutically useful characteristics. Another example is the peptide liraglutide, a GLP-1 agonist which has the sequence of GLP-1(7-37) with an arginine residue substituted for the native lysine at position 34, and with the sidechain of the lysine residue at position 26 being acylated by a hexadecanoyl group(palmitic acid) attached to the lysine though a glutamic acid spacer. Liraglutide has been developed for use as a once daily injectable drug for the treatment of type II diabetes. The same active ingredient as in liraglutide is marketed as Saxenda for the treatment of obesity (once daily injection). Semaglutide, a once weekly injectable analogue of GLP-1, has two amino acid substitutions compared to human GLP-1 (AIB8, Arg34) and is derivatized at lysine 26 with a linker and a C18 diacid fatty acid.

However, despite significant advances, the process of identifying substances useful as drugs remains a complex and, in many cases, unpredictable field. In order to be useful as therapeutic agents, compounds must possess a suitable range of properties. In addition to having good efficacy at the biological target of interest, compounds must have good in vivo pharmacokinetic properties, low toxicity and an acceptable side effect profile. For example, even with commercial agents such as liraglutide, side effects can include nausea and vomiting, and concerns have also been raised with regard to thyroid cancer and pancreatitis.

Thus, there remains a need for further compounds which are useful for the treatment of disorders and diseases such as diabetes and obesity. For example, it would be desirable to identify peptides having beneficial properties such as an improved activity profile, and/or which have reduced side effects. For example, it would be desirable for a peptide to be identified that reduces appetite and/or reduces food intake. Alternatively, or additionally, it would be desirable for a peptide to be identified that has these and other biological effects (for example the therapeutically useful biological effects described herein) for a sustained period. A compound that has a longer period of activity can be administered less frequently and at lower dose, which contributes to improved convenience for the subject, to fewer side effects and to lower cost.

### SUMMARY OF THE INVENTION

In a first aspect there is provided a compound of Formula (I):

(I)
[SEQ ID NO: 528]
Xaa1-AIB2-Xaa3-Gly4-Thr5-Phe6-Thr7-Ser8-Asp9-
Xaa10-Ser11-Lys12-Gln13-Leu14-Glu15-Glu16-Xaa17-
Xaa18-Val19-Xaa20-Xaa21-Phe22-Ile23-Glu24-Trp25-
Leu26-Lys27-Xaa28-Xaa29-Gly30-Pro31-Ser32-Xaa33-
Gly34-Xaa35-Xaa36-Pro37-Pro38-Xaa39-Xaa40-Xaa41-
Lys42

wherein:
Xaa1 is His, Tyr or Phe;
Xaa3 is His, Gln or Glu;
Xaa10 is Leu, Tyr or Val;
Xaa17 is Glu or Lys;
Xaa18 is Ala or Arg;
Xaa20 is Arg, Gln, Lys or His;
Xaa21 is Leu, Ala, Arg, Lys or Glu;
Xaa28 is Ala, Asn, Gln or His;
Xaa29 is Gly or Ala;
Xaa33 is Ser, His or Gln;
Xaa35 is Lys, Pro, His, Arg, Asn, Gln, Glu, Ser or Ala;
Xaa36 is Phe, His, Gln, Glu, Lys, Ser, Arg or absent
Xaa39 is Pro or Gly;
Xaa40 is Gly or absent;
Xaa41 is Lys or absent;
and the lysine residue at position 42 is substituted at its F-amino group with a group Y and Y is:
Z-Xaa43-Xaa44-
wherein:
Xaa43 is Asn or absent;
Xaa44 is His or absent;
and Z is a group of formula:

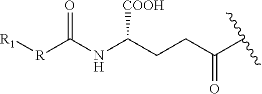

wherein R is a C₁₆-C₁₈ alkylene or alkenylene group; and
   R¹ is CO₂H;
      or a derivative of the compound; or a salt or solvate of the compound or the derivative.

Also provided herein is a composition comprising a compound, derivative, salt or solvate of the invention together with a pharmaceutically acceptable carrier and optionally a further therapeutic agent.

Also provided herein is a compound, derivative, salt or solvate of the invention, or a composition comprising such a compound, derivative, salt or solvate and a pharmaceutically acceptable carrier, for use as a medicament, e.g. for use in the prevention or treatment of diabetes, obesity, heart disease, stroke or non-alcoholic fatty liver disease, improving insulin release in a subject, improving carbohydrate metabolism in a subject, improving the lipid profile of a subject, reducing appetite, reducing food intake, reducing calorie intake, improving carbohydrate tolerance in a subject, and/or for use as a cytoprotective agent, for example in the prevention or treatment of Parkinsonism, Alzheimer's disease and other types of neural and cellular degeneration.

Also provided herein is a method of treating or preventing a disease or disorder or other non-desired physiological state in a subject comprising administration of a therapeutically effective amount of a compound, derivative, salt or solvate of the invention, or of a composition comprising such a compound, derivative, salt or solvate and a pharmaceutically acceptable carrier, e.g. in a method of treating or preventing diabetes, obesity, heart disease, stroke or non-alcoholic fatty liver disease in a subject, improving insulin release in a subject, improving carbohydrate metabolism in a subject, improving the lipid profile of a subject, improving carbohydrate tolerance in a subject, reducing appetite, reducing food intake, reducing calorie intake, and/or providing cytoprotection in a subject.

Also provided herein is a use of a compound, derivative, salt or solvate of the invention for the manufacture of a medicament for the prevention or treatment of diabetes, obesity, heart disease, stroke or non-alcoholic fatty liver disease, improving insulin release in a subject, improving carbohydrate metabolism in a subject, improving the lipid profile of a subject, improving carbohydrate tolerance in a subject, reducing appetite, reducing food intake, reducing calorie intake, and/or for use as a cytoprotective agent, for example in the prevention or treatment of Parkinsonism, Alzheimer's disease and other types of neural and cellular degeneration.

Also provided herein is a method of causing weight loss or preventing weight gain in a subject for cosmetic purposes comprising administration of an effective amount of a compound, derivative, salt or solvate of the invention.

### BRIEF DESCRIPTION OF THE DRAWINGS

FIG. 1 shows the amino acid sequences of example compounds of the invention. The compounds are presented with the N-terminal residue at the left-hand side of the table (signified by the column titled “1”). The amino acid sequence for each compound is set out over two horizontal pages. Results of feeding and receptor binding experiments are shown in the far right hand columns.

### SEQUENCES

The amino acid sequences herein are shown with the N-terminus to the left, and where sequences are set out across multiple lines, the N-terminus is to the top left. Unless indicated otherwise, the amino acid residues in the sequences are L-amino acids.

The amino acid sequences listed in the application are shown using standard letter abbreviations for amino acids. The unnatural amino acid 2-aminoisobutyric acid has its usual abbreviation ‘AIB’.

The specific sequences given herein relate to specific embodiments of the invention.

### DETAILED DESCRIPTION

In order to facilitate review of the various embodiments of this disclosure, the following explanations of specific terms are provided:

Animal: Living multi-cellular vertebrate organisms, a category that includes, for example, mammals and birds. The term mammal includes both human and non-human mammals. Similarly, the term “subject” includes both human and veterinary subjects. In preferred embodiments of the invention, the subject is a human subject.

Appetite: A natural desire, or longing for food. In one embodiment, appetite is measured by a survey to assess the desire for food. Increased appetite generally leads to increased feeding behaviour.

Appetite Suppressants: Compounds that decrease the desire for food. Commercially available appetite suppressants include, but are not limited to, amfepramone (diethylpropion), phentermine, mazindol and phenylpropanolamine fenfluramine, dexfenfluramine, and fluoxetine.

Body Mass Index (BMI): A mathematical formula for measuring body mass, also sometimes called Quetelet's Index. BMI is calculated by dividing weight (in kg) by height² (in metres²). The current standards for both men and women accepted as “normal” are a BMI of 20-24.9 kg/m². In one embodiment, a BMI of greater than 25 kg/m² can be used to identify an obese subject. Grade I obesity (which is sometimes referred to as being “overweight” rather than obesity) corresponds to a BMI of 25-29.9 kg/m². Grade II obesity corresponds to a BMI of 30-40 kg/m²; and Grade III obesity corresponds to a BMI greater than 40 kg/m² (Jequier, Am. J Clin. Nutr. 45:1035-47, 1987). Ideal body weight will vary among species and individuals based on height, body build, bone structure, and sex.

Cardioprotection refers to the protection of cardiac cells (and especially the myocardial cells) from apoptosis, necrotic cell death or degeneration (loss of function). Cardioprotection is most often required following myocardial infarction, but may also be used in subjects suffering from ischemic heart disease (for example angina)

Cytoprotection refers to the protection of cells from apoptosis, necrotic cell death or degeneration (loss of function).

Diabetes: A failure of cells to transport endogenous glucose across their membranes either because of an endogenous deficiency of insulin and/or a defect in insulin sensitivity. Diabetes is a chronic syndrome of impaired carbohydrate, protein, and fat metabolism owing to insufficient secretion of insulin or to target tissue insulin resistance. It occurs in two major forms: insulin-dependent diabetes mellitus (IDDM, type I) and non-insulin dependent diabetes mellitus (NIDDM, type II) which differ in etiology, pathology, genetics, age of onset, and treatment.

The two major forms of diabetes are both characterized by an inability to deliver insulin in an amount and with the precise timing that is needed for control of glucose homeostasis. Diabetes type I, or insulin dependent diabetes mellitus (IDDM) is caused by the destruction of p cells, which results in insufficient levels of endogenous insulin. Diabetes type II, or non-insulin dependent diabetes, results from a defect in both the body's sensitivity to insulin, and a relative deficiency in insulin production.

Energy Metabolism: The body has to expend a certain amount of energy to maintain normal metabolism. In civilized man this is often set at about 2,800 Calories daily. If food consumption does not provide this, weight loss results. However, energy metabolism is also regulated, and, for example, administration of glucagon is thought to increase the metabolic rate so that a greater food intake is required to achieve energy balance and maintain weight. Thus, if food intake is maintained at the usual level, but energy metabolism is increased, weight loss will result.

Food intake: The amount of food consumed by an individual. Food intake can be measured by volume or by weight. For example, food intake may be the total amount of food consumed by an individual. In a feeding experiment, ‘Food Intake’ is the weight of a standardised chow consumed by an animal in a 24 hour period. Or food intake may be the amount of proteins, fat, carbohydrates, cholesterol, vitamins, minerals, or any other food component, of the individual. “Protein intake” refers to the amount of protein consumed by an individual. Similarly, “fat intake,” “carbohydrate intake,” “cholesterol intake,” “vitamin intake,” and “mineral intake” refer to the amount of proteins, fat, carbohydrates, cholesterol, vitamins, or minerals consumed by an individual.

GLP-1: Glucagon-like peptide 1 (GLP-1) is derived from the transcription product of the proglucagon gene. The biologically active forms of GLP-1 are truncated forms known as GLP-1(7-37) and GLP-1(7-36)-NH₂ (the designation —NH₂ designates an amino acid sequence in which the C-terminal amino acid has a —C(O)NH₂ group in place of a carboxylic acid group).

The sequence of human GLP-1(7-37) is His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly. [SEQ ID NO: 529]

The sequence of human GLP-1(7-36)-NH₂ is His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-CONH₂. [SEQ ID NO: 530]

Glucagon: Glucagon is a peptide derived from the proglucagon gene. It is a 29-amino acid polypeptide in humans and has the sequence:

[SEQ ID NO: 531]
His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Met-Asn-Thr

Neuroprotection refers to the protection of neurons within the nervous system (preferably within the central nervous system) from apoptosis, necrotic cell death or degeneration (loss of function). Neuroprotective treatments, including those relating to various aspects of the present invention may be required following a brain injury (for example those following physical trauma or non-traumatic injury such as stroke, brain tumours, infection, poisoning, hypoxia, ischemia, encephalopathy or substance abuse). Neuroprotective treatments, including those relating to various aspects of the present invention may also be indicated in subjects having a chronic neurodegenerative disease such as Alzheimer's disease, Parkinson's disease, Gehrig's disease or Huntington's disease.

Normal Daily Diet: The average food intake for an individual of a given species. A normal daily diet can be expressed in terms of caloric intake, protein intake, carbohydrate intake, and/or fat intake. A normal daily diet in humans generally comprises the following: about 2,000, about 2,400, or about 2,800 to significantly more calories. In addition, a normal daily diet in humans generally includes about 12 g to about 45 g of protein, about 120 g to about 610 g of carbohydrate, and about 11 g to about 90 g of fat. A low calorie diet would be no more than about 85%, and preferably no more than about 70%, of the normal caloric intake of a human individual.

In animals, the caloric and nutrient requirements vary depending on the species and size of the animal. For example, in cats, the total caloric intake per pound, as well as the percent distribution of protein, carbohydrate and fat varies with the age of the cat and the reproductive state. A general guideline for cats, however, is 40 cal/lb/day (18.2 cal/kg/day). About 30% to about 40% should be protein, about 7% to about 10% should be from carbohydrate, and about 50% to about 62.5% should be derived from fat intake. One of skill in the art can readily identify the normal daily diet of an individual of any species.

Obesity: A condition in which excess body fat may put a person at health risk (see Barlow and Dietz, Pediatrics 102:E29, 1998; National Institutes of Health, National Heart, Lung, and Blood Institute (NHLBI), Obes. Res. 6 (suppl. 2):51S-209S, 1998). Excess body fat is a result of an imbalance of energy intake and energy expenditure. For example, the Body Mass Index (BMI) may be used to assess obesity. In one commonly used convention, a BMI of 25.0 kg/m² to 29.9 kg/m² is overweight, while a BMI of 30 kg/m² or greater is obese.

In another convention, waist circumference is used to assess obesity. In this convention, in men a waist circumference of 102 cm or more is considered obese, while in women a waist circumference of 89 cm or more is considered obese. Strong evidence shows that obesity affects both the morbidity and mortality of individuals. For example, an obese individual is at increased risk for heart disease, non-insulin dependent (type 2) diabetes, hypertension, stroke, cancer (e.g. endometrial, breast, prostate, and colon cancer), dyslipidemia, gall bladder disease, sleep apnea, reduced fertility, and osteoarthritis, amongst others (see Lyznicki et al., Am. Fam. Phys. 63:2185, 2001).

Overweight: An individual who weighs more than their ideal body weight. An overweight individual can be obese but is not necessarily obese. For example, an overweight individual is any individual who desires to decrease their weight. In one convention, an overweight individual is an individual with a BMI of 25.0 kg/m² to 29.9 kg/m².

Oxyntomodulin (OXM): Oxyntomodulin is a 37 amino acid peptide member of the glucagon superfamily comprising the entire 29 amino acid sequence of glucagon, with an eight amino acid carboxy terminal extension, resulting from the tissue-specific processing of the pre-pro-glucagon precursor in the brain and gut. The human OXM sequence is as follows:

[SEQ ID NO: 532]
His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Met-Asn-Thr-Lys-Arg-Asn-Arg-Asn-Asn-Ile-
Ala

PEGylation and PEGylated: PEGylation refers to the process of reacting a poly(alkylene glycol), preferably an activated poly(alkylene glycol) to form a covalent bond. A facilitator may be used, for example an amino acid, e.g. lysine. Although “PEGylation” is often carried out using poly(ethylene glycol) or derivatives thereof, such as methoxy poly(ethylene glycol), the term is not limited herein to the use of methoxy poly(ethylene glycol) but also includes the use of any other useful poly(alkylene glycol), for example poly(propylene glycol). The term PEGylated refers to a compound containing such a poly(alkylene glycol) group.

pI: pI is an abbreviation for isoelectric point. An alternative abbreviation sometimes used is IEP. It is the pH at which a particular molecule carries no net electric charge. At a pH below its pI a protein or peptide carries a net positive charge. At a pH above its pI a protein or peptide carries a net negative charge. Proteins and peptides can be separated according to their isoelectric points using a technique called isoelectric focusing which is an electrophoretic method that utilises a pH gradient contained within a polyacrylamide gel.

Peripheral administration: Administration outside of the central nervous system. Peripheral administration does not include direct administration to the brain. Peripheral administration includes, but is not limited to intravascular, intramuscular, subcutaneous, inhalation, oral, rectal, transdermal or intra-nasal administration.

Polypeptide: A polymer in which the monomers are amino acid residues which are joined together through amide bonds. Unless dictated otherwise by context, the terms “polypeptide”, “peptide”, or “protein” as used herein encompass any amino acid sequence and include modified sequences such as glycoproteins. The term “polypeptide” covers naturally occurring proteins, as well as those which are recombinantly or synthetically produced. The term “polypeptide fragment” refers to a portion of a polypeptide, for example a fragment which exhibits at least one useful sequence in binding a receptor. The term “functional fragments of a polypeptide” refers to all fragments of a polypeptide that retain an activity of the polypeptide. Biologically functional peptides can also include fusion proteins, in which the peptide of interest has been fused to another peptide that does not decrease its desired activity.

Subcutaneous administration: Subcutaneous administration is administration of a substance to the subcutaneous layer of fat which is found between the dermis of the skin and the underlying tissue. Subcutaneous administration may be by an injection using a hypodermic needle fitted, for example, to a syringe or a “pen” type injection device. Other administration methods may be used for example microneedles. Injection with a hypodermic needle typically involves a degree of pain on behalf of the recipient. Such pain may be masked by use of a local anaesthetic or analgesic. However, the usual method used to reduce the perceived pain of injections is to merely distract the subject immediately prior to and during the injection. Pain may be minimised by using a relatively small gauge hypodermic needle, by injecting a relatively small volume of substance and by avoiding excessively acidic or alkali compositions which may cause the subject to experience a “stinging” sensation at the injection site. Compositions having a pH of between pH 4 and pH 10 are usually regarded as tolerably comfortable.

Therapeutically effective amount: A dose sufficient to prevent advancement, or to cause regression of a disorder, or which is capable of relieving a sign or symptom of a disorder, or which is capable of achieving a desired result. In some embodiments, a therapeutically effective amount of a compound of the invention is an amount sufficient to inhibit or halt weight gain, or an amount sufficient to decrease appetite.

### Compounds of the Invention

The present inventors have found that example compounds of the invention have properties including causing weight loss in vivo. The compounds also have a long half-life in the blood, meaning that they can be administered at a conveniently low frequency. In these respects, the compounds of the invention have an improved profile in comparison to the known compound semaglutide.

Compared with GLP-1 and derivatives of GLP-1 that have gone before, the compounds of the invention are extended at the C-terminus with particular residues, including that there is a lysine residue at position 42 and including that that lysine residue is functionalised in a particular way, to include the group R—R¹ where R¹ is CO₂H. These are functionalisations that have not previously been investigated and the beneficial properties found by the current inventors have not previously been seen.

As described above, compounds of the invention have Formula (I):

(I)
[SEQ ID NO: 528]
Xaa1-AIB2-Xaa3-Gly4-Thr5-Phe6-Thr7-Ser8-Asp9-
Xaa10-Ser11-Lys12-Gln13-Leu14-Glu15-Glu16-
Xaa17-Xaa18-Val19-Xaa20-Xaa21-Phe22-Ile23-Glu24-
Trp25-Leu26-Lys27-Xaa28-Xaa29-Gly30-Pro31-Ser32-
Xaa33-Gly34-Xaa35-Xaa36-Pro37-Pro38-Xaa39-Xaa40-
Xaa41-Lys42;

wherein
Xaa1 is His, Tyr or Phe;
Xaa3 is His, Gln or Glu;
Xaa10 is Leu, Tyr or Val;
Xaa17 is Glu or Lys;
Xaa18 is Ala or Arg;
Xaa20 is Arg, Gln, Lys or His;
Xaa21 is Leu, Ala, Arg, Lys or Glu;
Xaa28 is Ala, Asn, Gln or His;
Xaa29 is Gly or Ala;
Xaa33 is Ser, His or Gln;
Xaa35 is Lys, Pro, His, Arg, Asn, Gln, Glu, Ser or Ala;
Xaa36 is Phe, His, Gln, Glu, Lys, Ser, Arg or absent
Xaa39 is Pro or Gly;
Xaa40 is Gly or absent;
Xaa41 is Lys or absent;
and the lysine residue at position 42 is substituted at its F-amino group with a group Y and Y is:
Z-Xaa43-Xaa44-
wherein:
Xaa43 is Asn or absent;
Xaa44 is His or absent;
and Z is a group of formula:

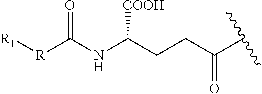

wherein R is a C₁₆-C₁₈ alkylene or alkenylene group; and
   R¹ is CO₂H.

Within the first portion of the molecule (Xaa1-Lys42), it is preferred that:
Xaa17 is Lys;
Xaa18 is Ala or Arg;
Xaa20 is Arg or His;
Xaa21 is Leu or Glu;
Xaa28 is Ala, Gln or His;
Xaa29 is Gly;
Xaa33 is Ser
Xaa35 is Lys, Glu, His, Gln or Asn;
Xaa36 is absent;
Xaa39 is Pro; and
Xaa41 is Lys.

In a more preferred embodiment, in the first portion of the molecule (Xaa1-Lys42):
Xaa1 is His or Phe;
Xaa3 is His or Glu;
Xaa10 is Tyr or Val;
Xaa17 is Lys;
Xaa18 is Ala or Arg;
Xaa20 is Arg or His;
Xaa21 is Glu;
Xaa28 is Ala or Gln;
Xaa29 is Gly;
Xaa33 is Ser
Xaa35 is Lys;
Xaa36 is absent;
Xaa39 is Pro;
Xaa40 is Gly; and
Xaa41 is Lys.

Considering the residues in turn, Xaa1 is selected from His, Tyr and Phe. For example, Xaa1 is selected from His and Tyr, or alternatively Xaa1 is selected from His and Phe. For example, Xaa1 is His. Alternatively, Xaa1 is Phe. In an especially preferred embodiment, Xaa1 is Phe. It has been found that compounds of the invention with Phe at the N-terminus (in particular ones that also have Xaa3=Glu) have a GLP-1 receptor bias. Compounds with Phe at the N-terminus have been seen to fully activate the intracellular cAMP system, in a manner similar to native GLP1, whilst being weaker than GLP1 at activating the intracellular beta arrestin messenger system, so there is a bias at the GLP1 receptor. In some circumstances, this GLP1 receptor bias is an advantage in prolonging the action at the GLP1 receptor and producing relative enhancement of the action on the beta cell in releasing insulin while not enhancing other dose limiting effects such as inhibition of food intake and food aversion.

Xaa3 is selected from His, Gln and Glu. Preferably, Xaa3 is selected from His and Glu. For example, Xaa3 is His. Alternatively, Xaa3 is Glu. In an especially preferred embodiment, Xaa3 is Glu.

Xaa10 is selected from Leu, Tyr and Val. Preferably, Xaa10 is Tyr or Val, for example Xaa10 is Tyr. Alternatively, Xaa10 is Val.

Xaa17 is selected from Glu or Lys. Preferably, Xaa17 is Lys.

Xaa18 is selected from Ala or Arg. For example, Xaa18 is Ala. Alternatively, Xaa18 is Arg. In an especially preferred embodiment, Xaa18 is Arg.

Xaa20 is selected from Arg, Gln, Lys and His. Preferably, Xaa20 is Arg or His, for example Xaa20 is Arg. Alternatively, Xaa20 is His. In an especially preferred embodiment, Xaa20 is Arg.

Xaa21 is selected from Leu, Ala, Arg, Lys and Glu. Preferably, Xaa21 is Leu or Glu, for example, Xaa21 is Glu. Alternatively, Xaa21 is Leu. In an especially preferred embodiment, Xaa21 is Glu.

Xaa28 is selected from Ala, Asn, Gln and His. Preferably, Xaa28 is selected from Ala, Gln and His. In an especially preferred embodiment, Xaa28 is Ala or Gln, for example Xaa28 is Ala. Alternatively, Xaa28 is Gln.

Xaa29 is selected from Gly and Ala. Preferably, Xaa29 is Gly.

Xaa33 is selected from Ser, His and Gln. For example, Xaa33 is selected from Ser and His, or alternatively Xaa33 is selected from Ser and Gln. Preferably, Xaa33 is Ser.

Xaa35 is selected from Lys, Pro, His, Arg, Asn, Gln, Glu, Ser and Ala. Preferably, Xaa35 is selected from Lys, Gln, Glu, His and Asn. In an especially preferred embodiment, Xaa35 is Lys.

Xaa36 is selected from Phe, His, Gln, Glu, Lys, Ser, Arg, or Xaa36 is absent. Preferably, Xaa36 is absent.

Xaa39 is selected from Pro or Gly. Preferably, Xaa39 is Pro.

Xaa40 is Gly, or Xaa40 is absent. Preferably, Xaa40 is Gly.

Xaa41 is Lys, or Xaa41 is absent. Preferably, Xaa41 is Lys.

In a preferred embodiment, Xaa36 is absent, and Xaa37-Xaa41 is Pro37-Pro38-Pro39-Gly40-Lys41 [SEQ ID NO: 533]. In an alternative preferred embodiment, Xaa36 is absent, Xaa40 is absent and Xaa37-Xaa41 is Pro37-Pro38-Pro39-Lys41 [SEQ ID NO: 534].

### Group Y

In the compounds of the invention, the F-amino group on Lys42 is attached to the acid group of the Xaa44 residue of the Y portion of the molecule, such that the bond is an amide bond. If Xaa44 or Xaa43 is absent, then the F-amino group on Lys42 is attached to the acid group of the next residue that is present: the acid group of the Z group.

In a lysine residue, the F-amino group is the amino group that is attached to the 6-carbon. Following standard IUPAC nomenclature, the atoms in lysine are numbered as follows, indicating the carbon atom numbering and also the a to F positions:

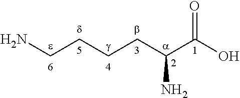

The F-amino group on Lys42 that is referred to herein is the amino group on the C-6 carbon atom as indicated.

Within the Y portion of the molecule:
Xaa43 may be Asn or absent; and
Xaa44 may be His or absent.

For example, Xaa43 is Asn and Xaa44 is His; or Xaa43 is Asn and Xaa44 is absent; or Xaa43 is absent and Xaa44 is His; or both Xaa43 and Xaa44 are absent. In a preferred embodiment, Xaa43 is absent and Xaa44 is absent.

For example, Y is:
Z-Asn-His-;
Z-Asn-;
Z-His-; or
Z—.

### Group Z

Within the Z portion of the molecule, group R is an alkylene or alkenylene chain, which is linked at one end to residue Xaa43 (or, if Xaa43 is absent, Xaa44, or if both Xaa43 and Xaa44 are absent, Lys42) by the Glu residue through an amide bond. At its other end, the R alkylene or alkenylene chain is linked to the R¹ acid group (CO₂H).

Generally, R has an even number of carbon atoms. For example, R can be an alkylene or alkenylene chain that is found in naturally-occurring fatty acids. The root fatty acid has a chain length two higher than the number of carbon atoms in the R an alkylene or alkenylene chain.

R is a C₁₆-C₁₈ alkylene or alkenylene group. For example, R is straight chain alkylene or alkenylene group. For example, R is a C₁₆ or C₁₈ straight chain alkylene group. For example, when R is a C₁₆, group, it can be provided by an octadecanedioic acid moiety. For example, when R is a Cis, group, it can be provided by an eicosanedioic acid moiety.

In a preferred embodiment, R is a C₁₈ alkylene group.

In an embodiment, it is preferred that:
Xaa17 is Lys;
Xaa20 is Arg or His;
Xaa21 is Leu or Glu;
Xaa28 is Ala, Gln or His;
Xaa29 is Gly;
Xaa33 is Ser
Xaa35 is Lys, Glu, His, Gln or Asn;
Xaa36 is absent;
Xaa39 is Pro;
Xaa41 is Lys;
Xaa43 is absent;
Xaa44 is absent; and
R is a C₁₈ alkylene group.

In a more preferred embodiment:
Xaa1 is His or Phe;
Xaa3 is His or Glu;
Xaa10 is Tyr or Val;
Xaa17 is Lys;
Xaa18 is Ala or Arg;
Xaa20 is Arg or His;
Xaa21 is Glu;
Xaa28 is Ala or Gln;
Xaa35 is Lys; and
Xaa40 is Gly.

For example, in such an embodiment:
Xaa1 is Phe;
Xaa3 is Glu;
Xaa10 is Val;
Xaa18 is Arg;
Xaa20 is Arg; and
Xaa28 is Gln.

For example, such a compound can be a compound of SEQ ID No: 502.

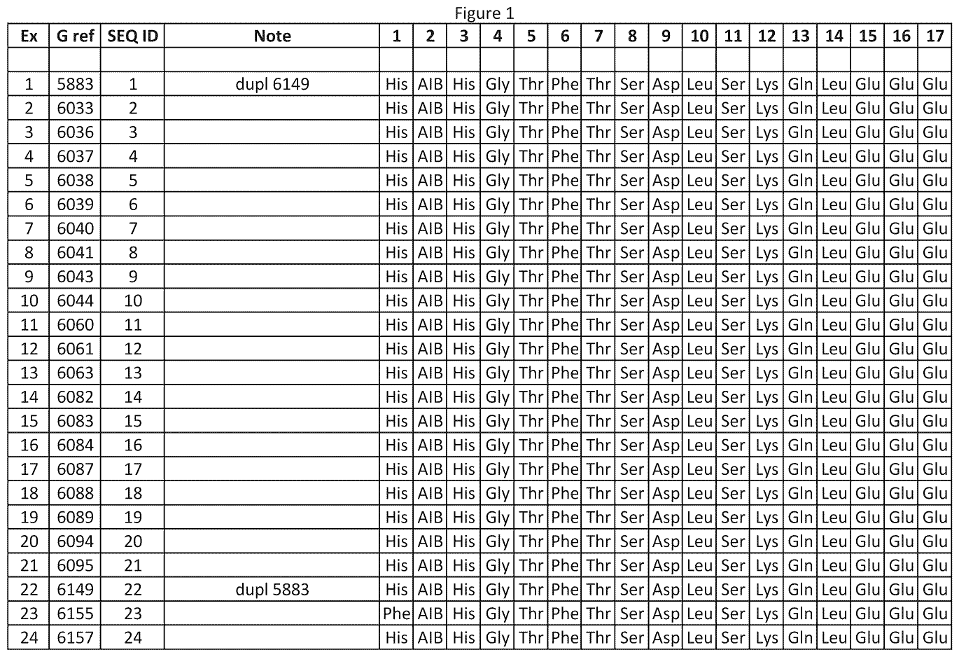

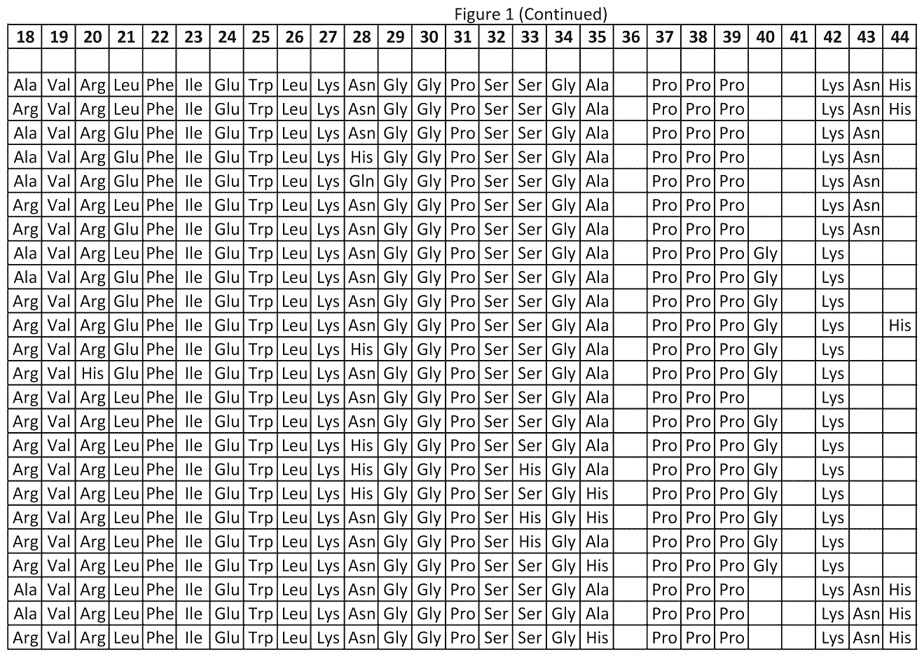

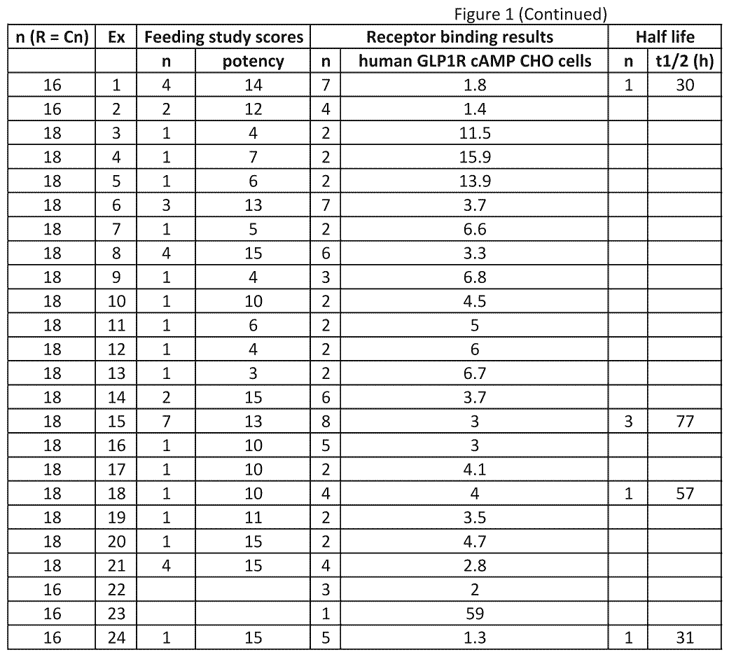

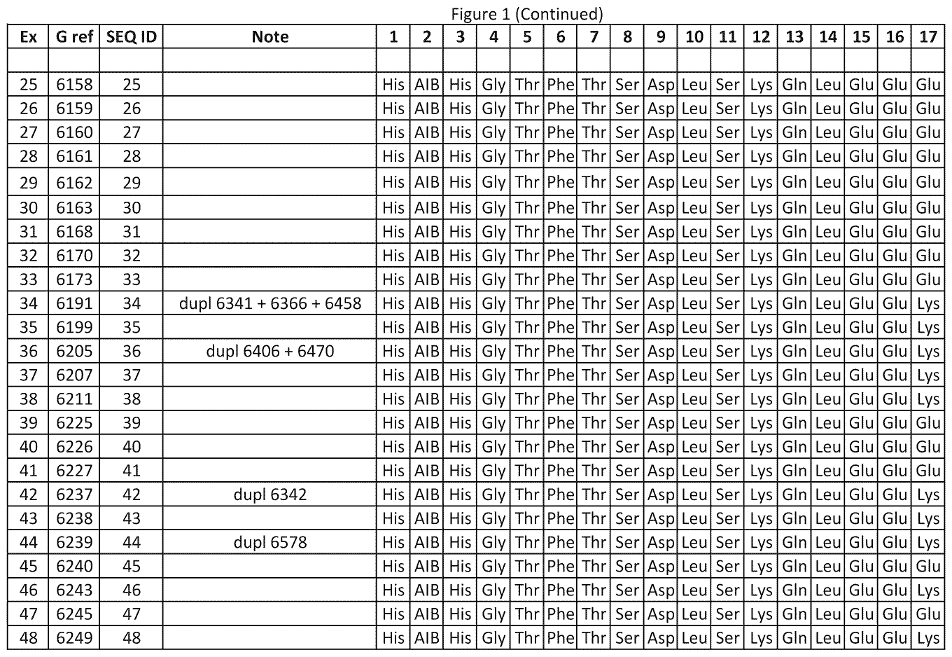

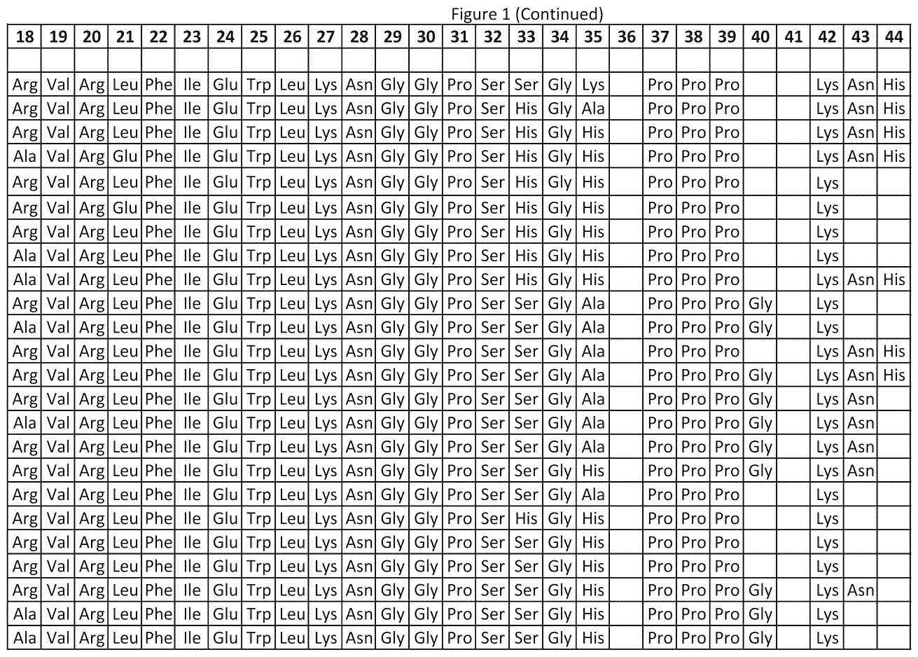

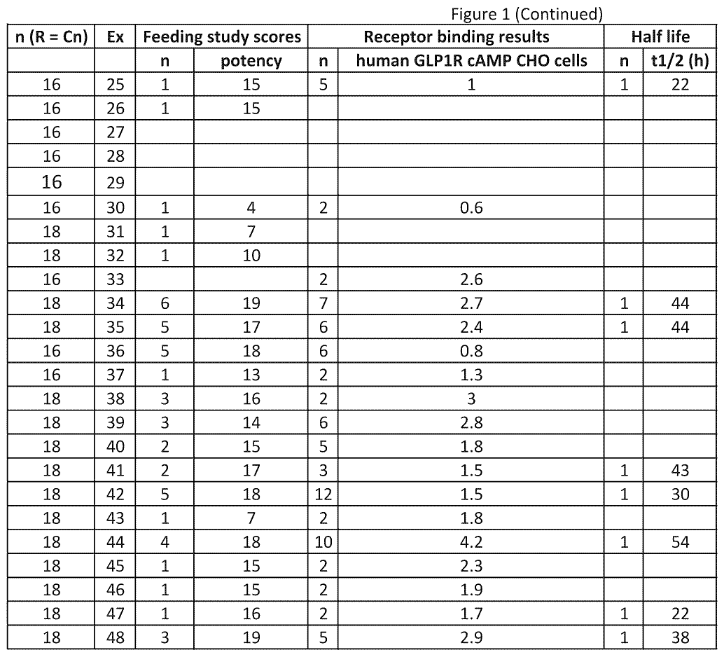

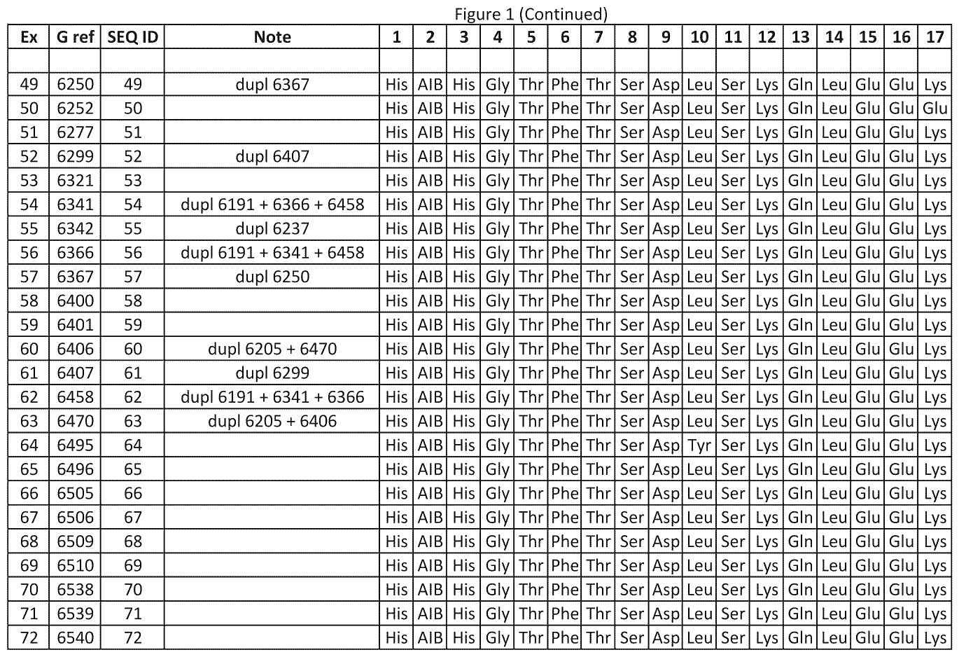

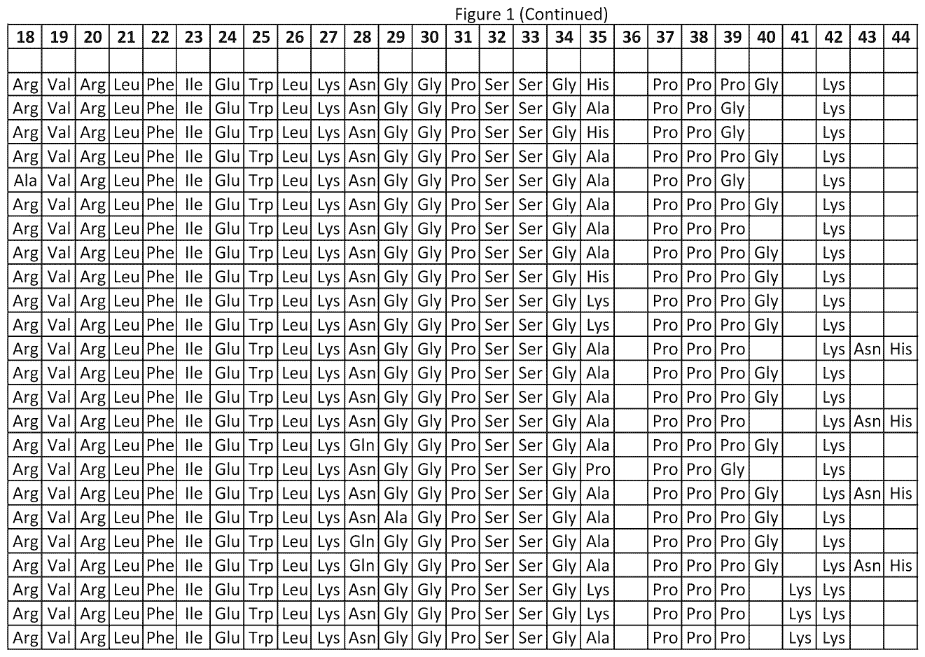

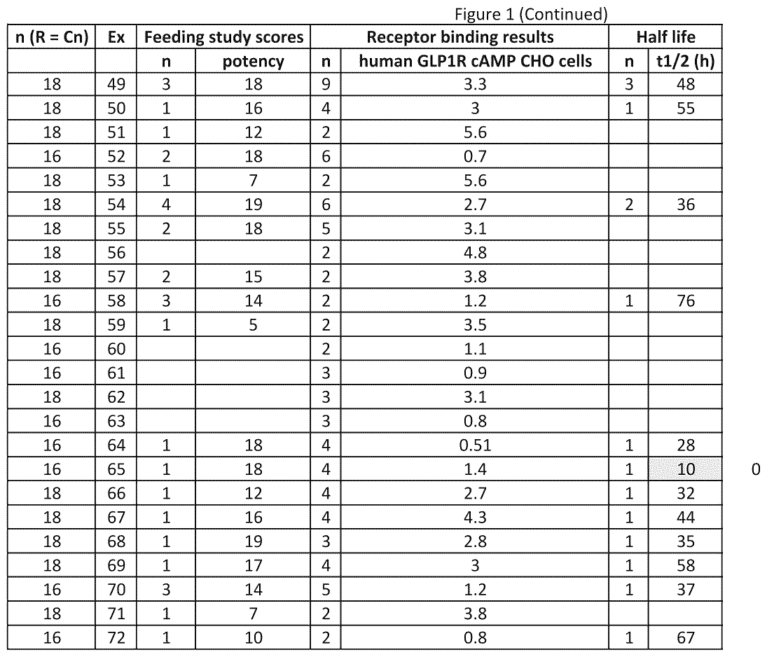

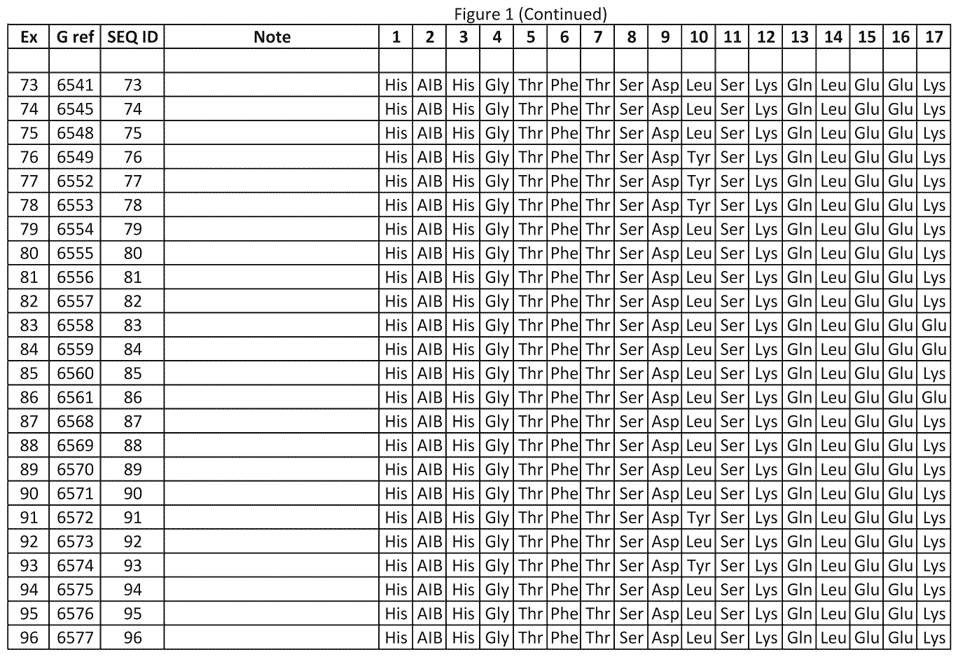

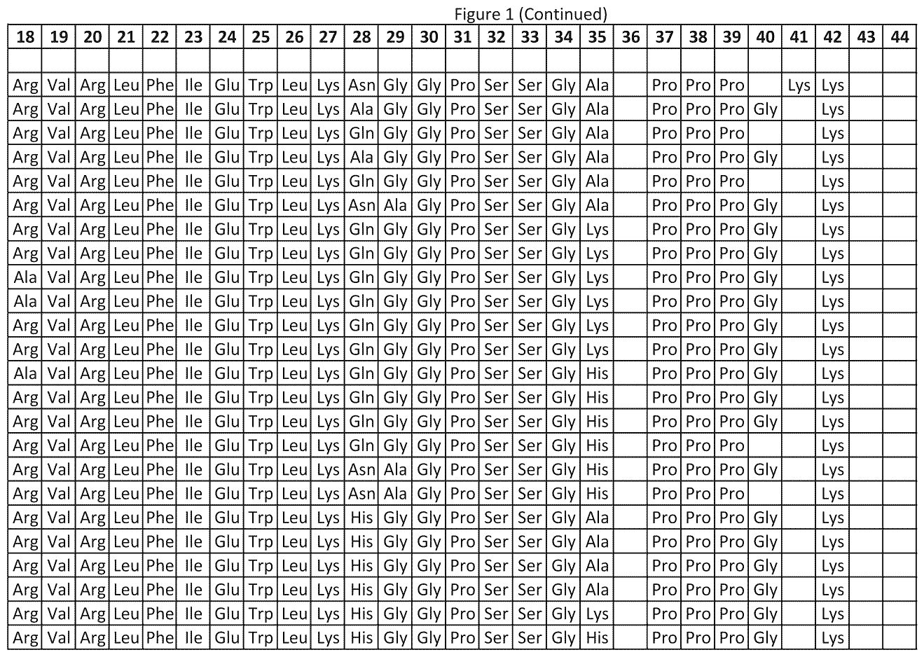

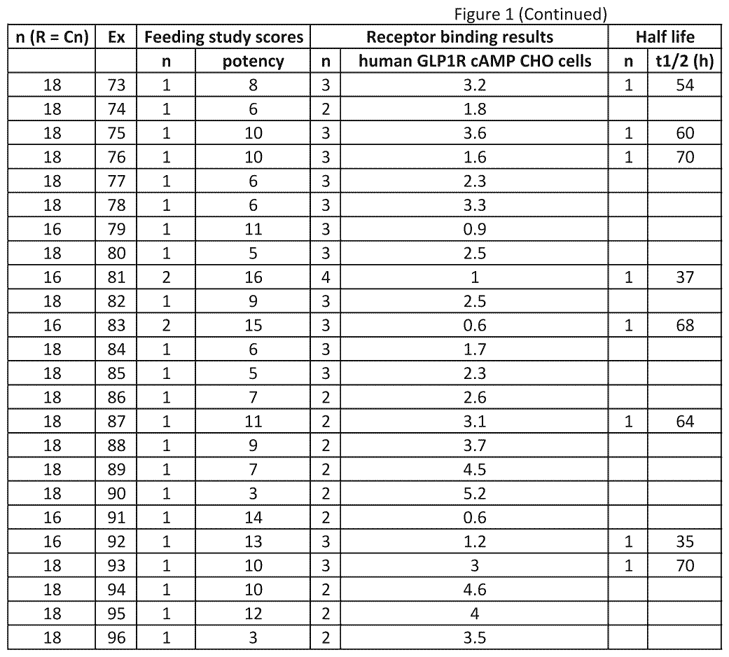

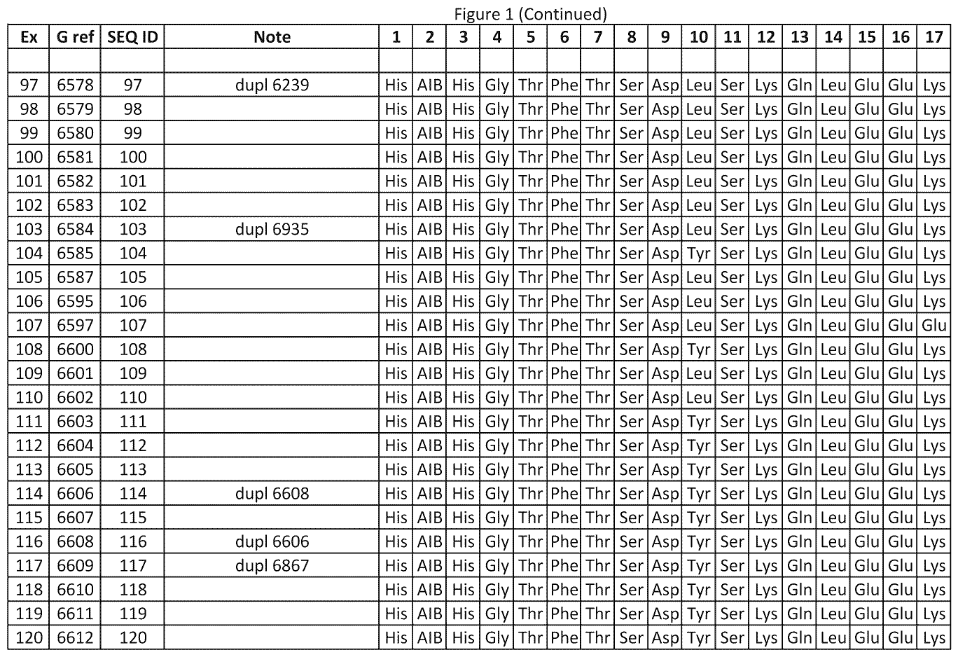

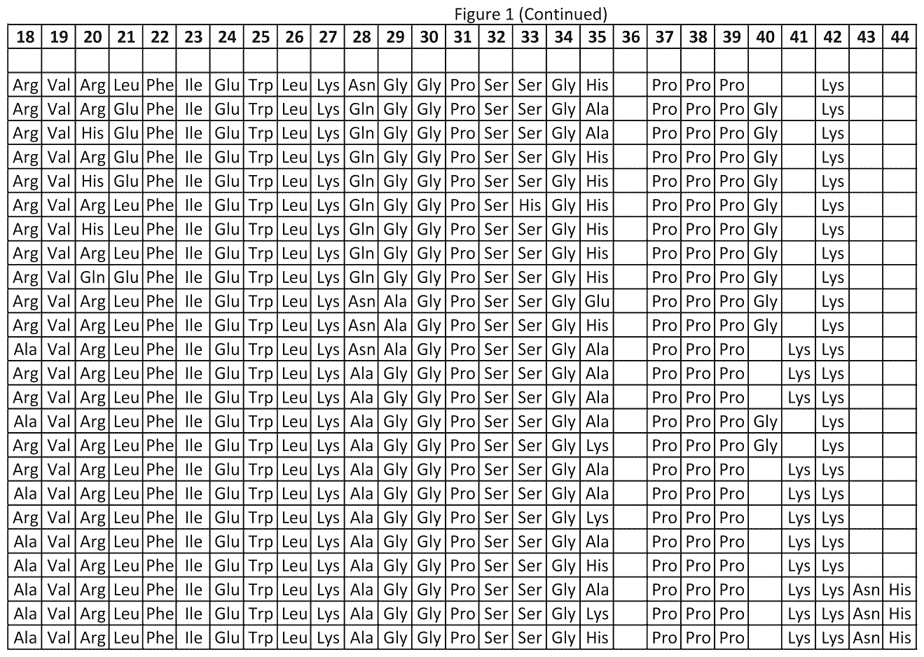

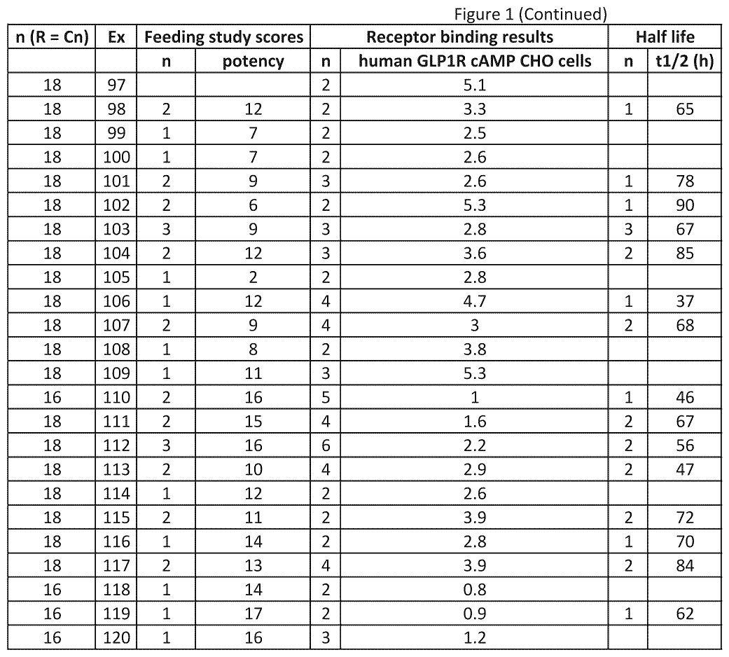

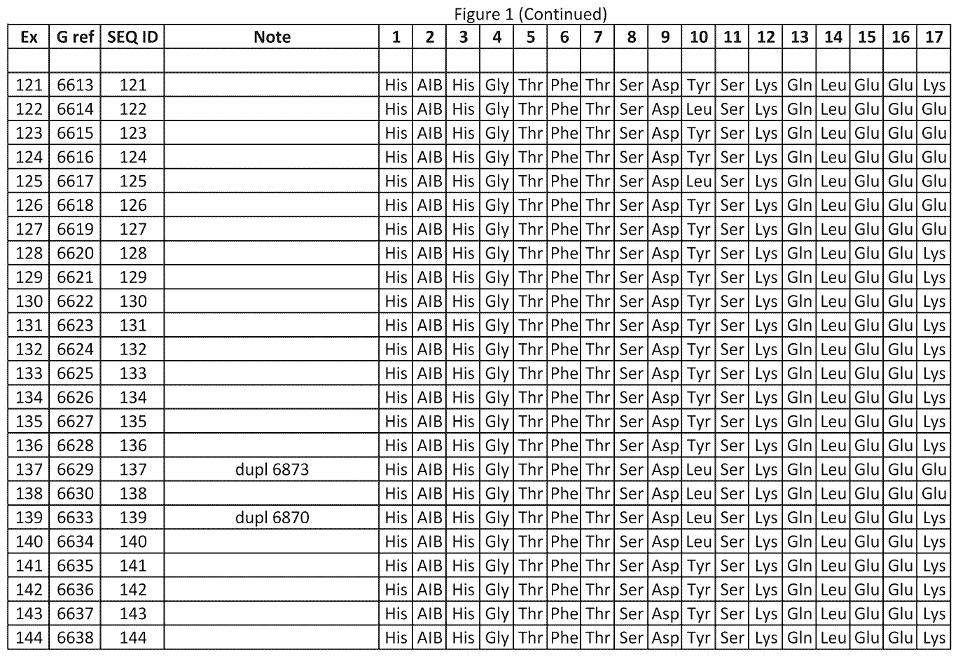

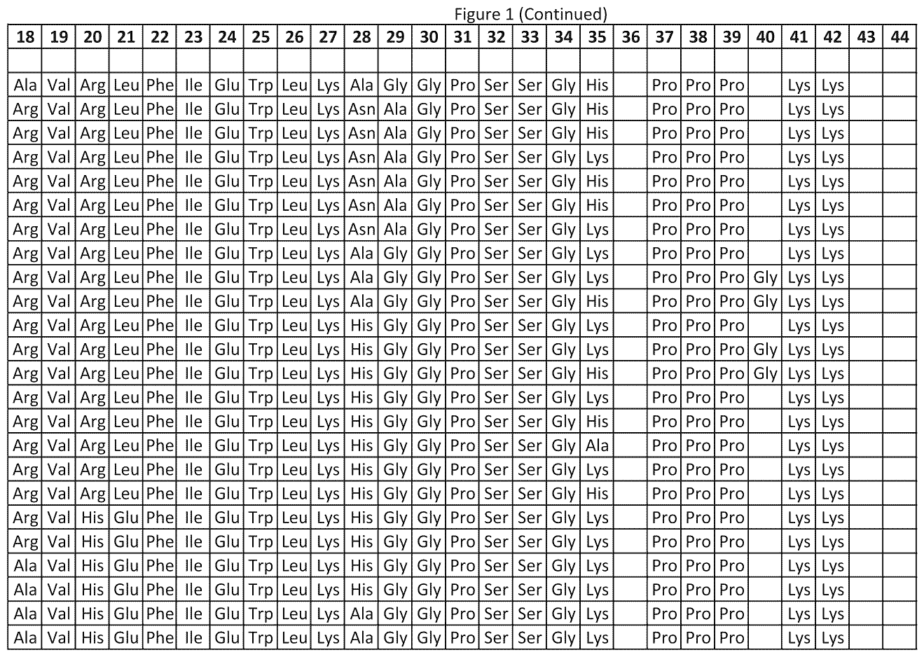

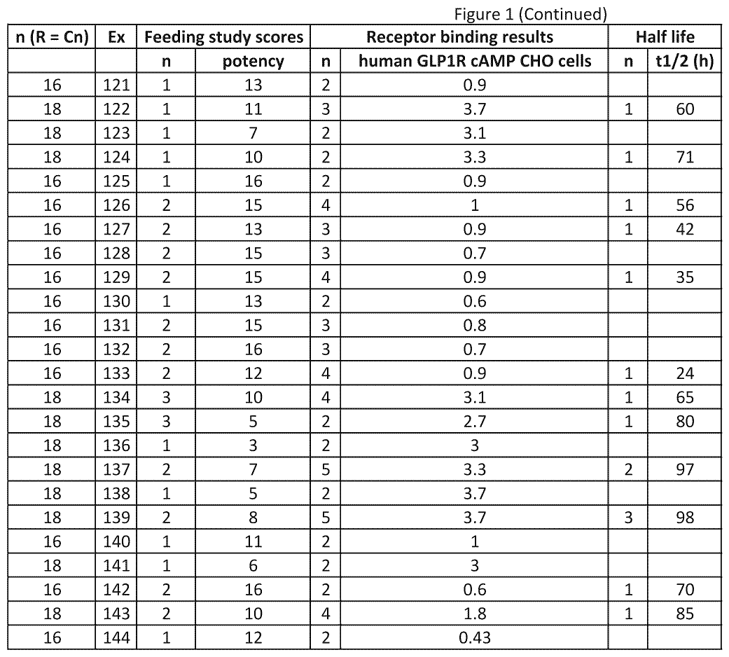

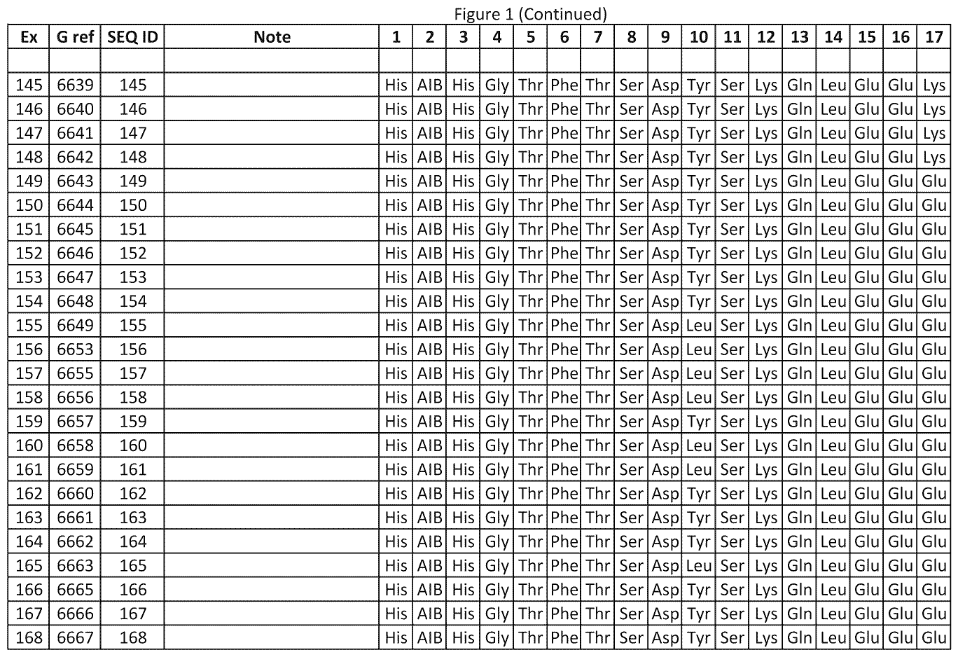

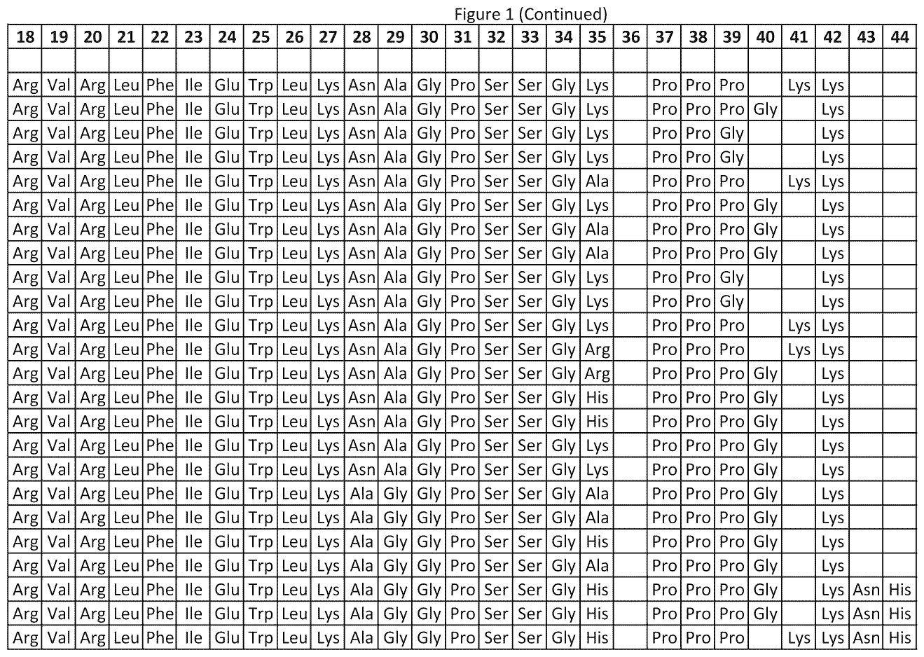

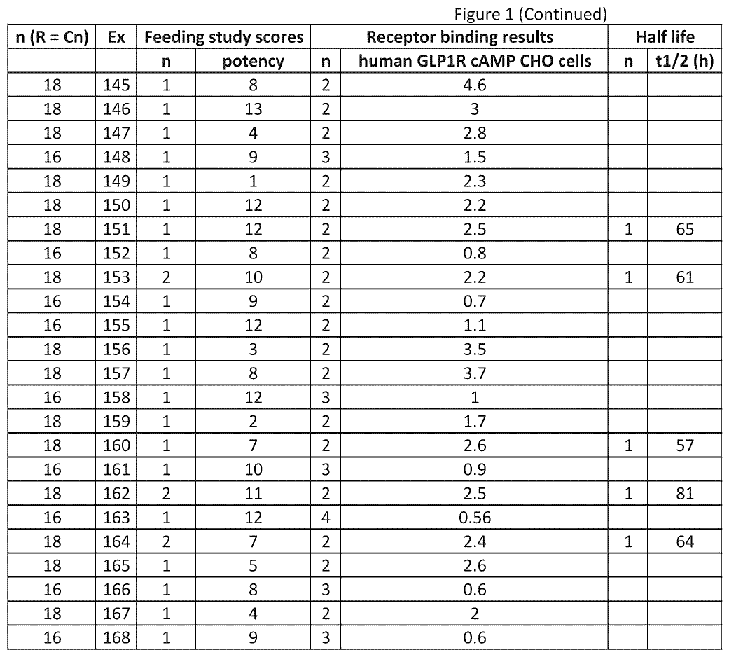

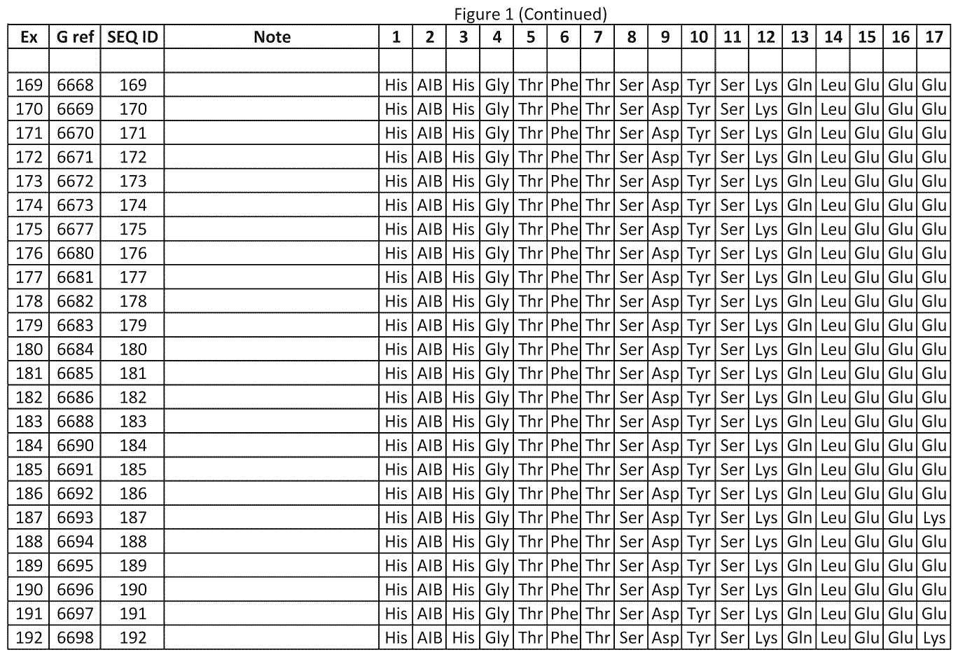

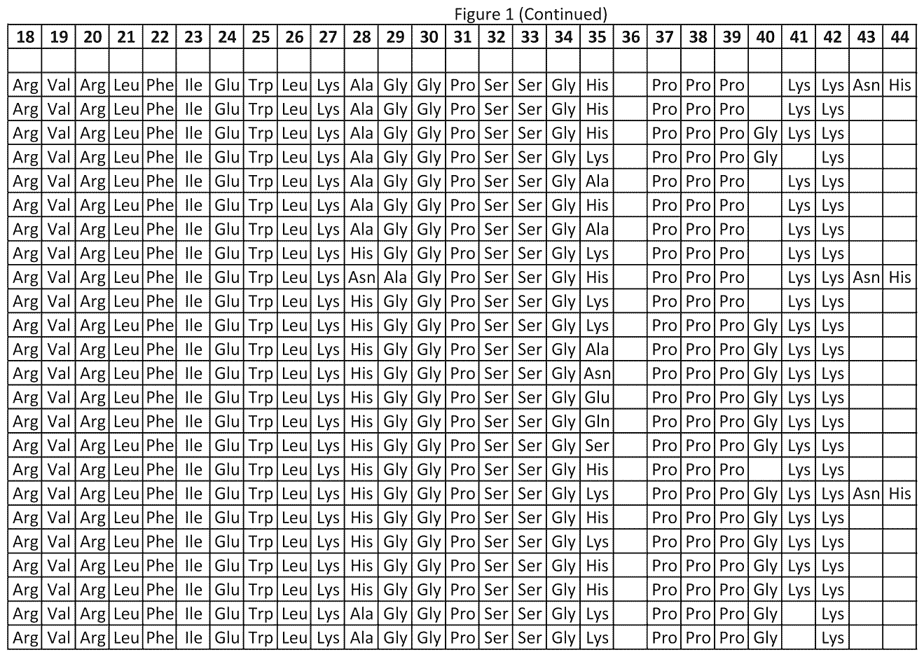

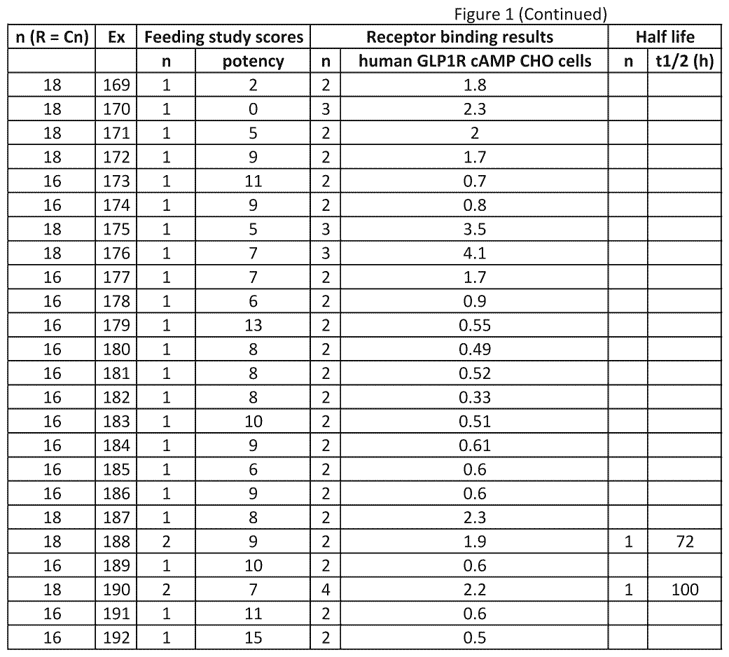

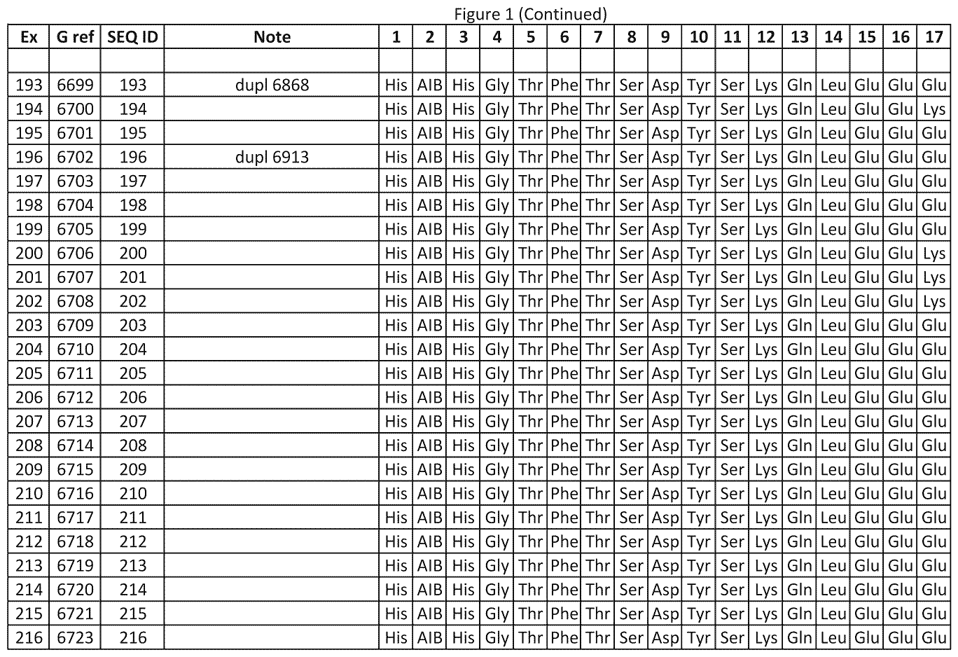

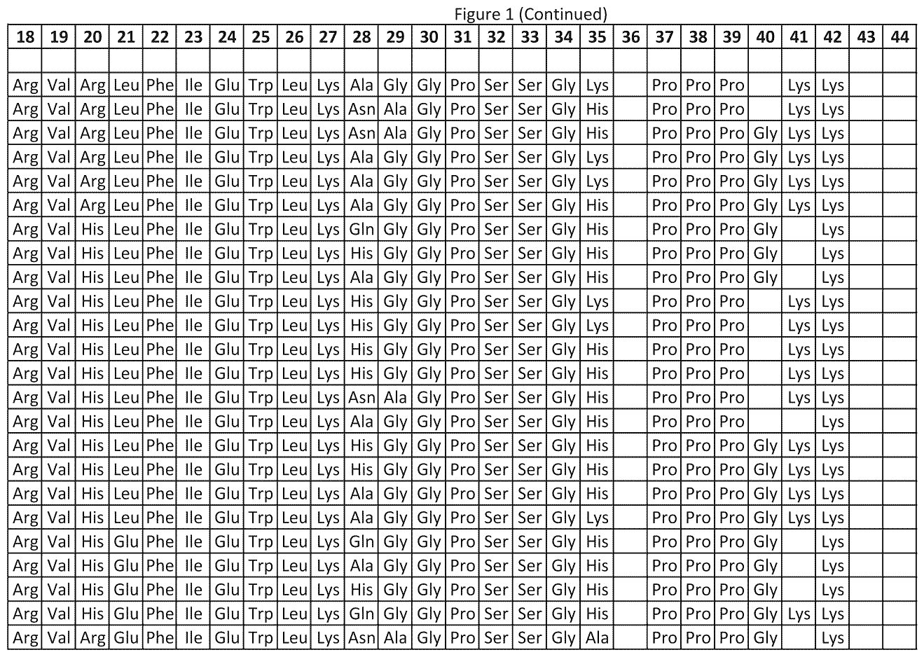

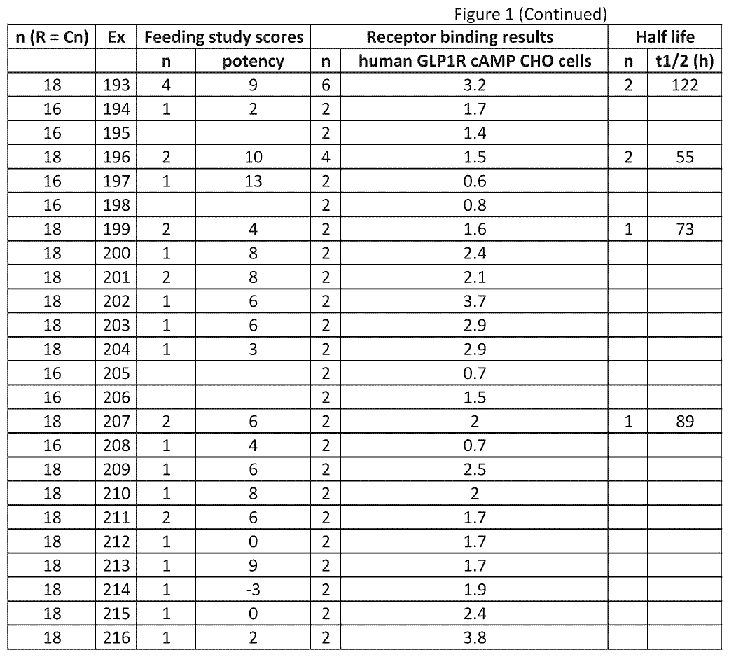

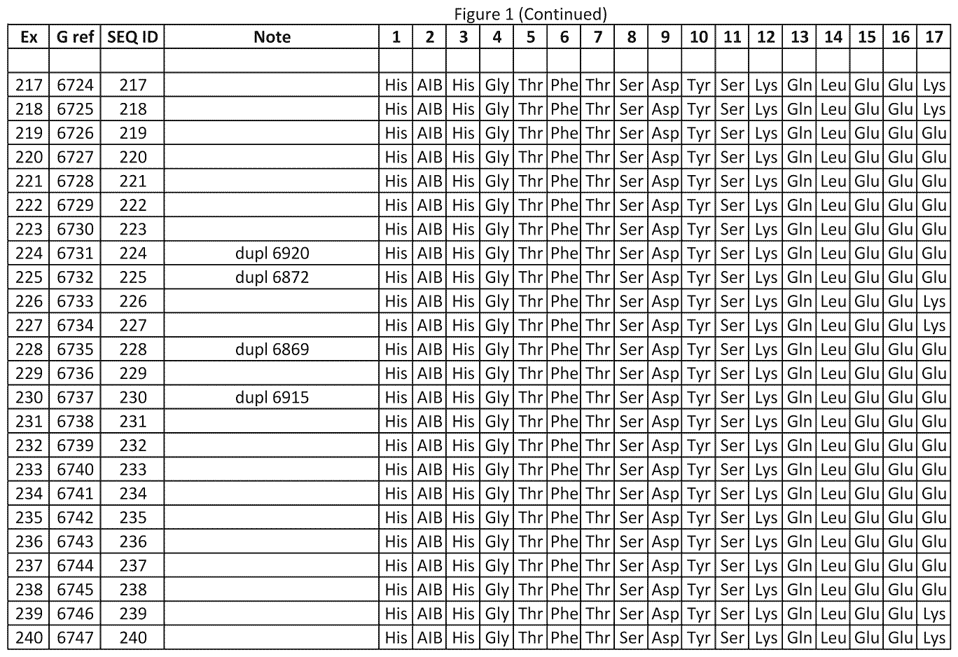

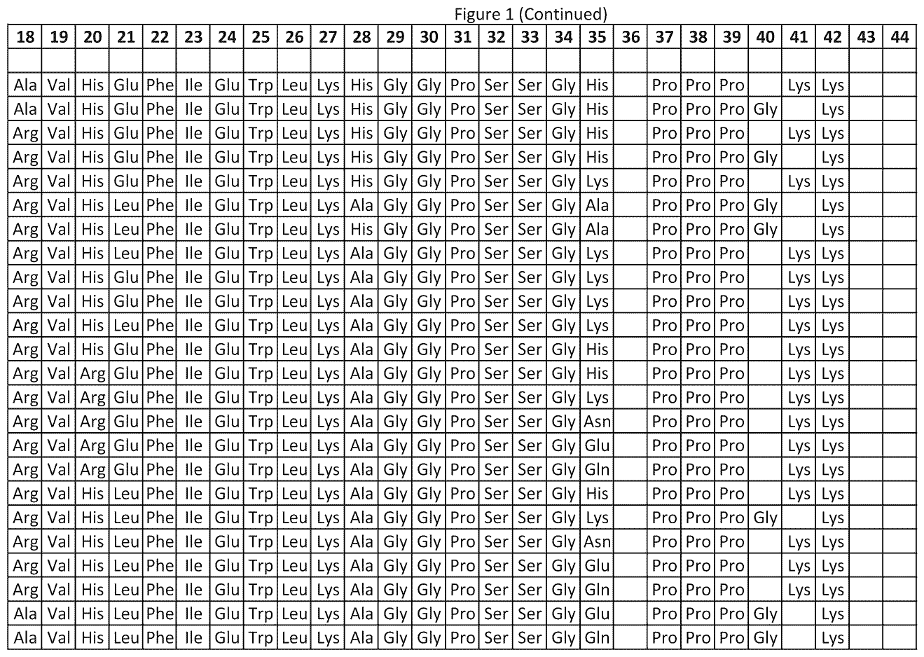

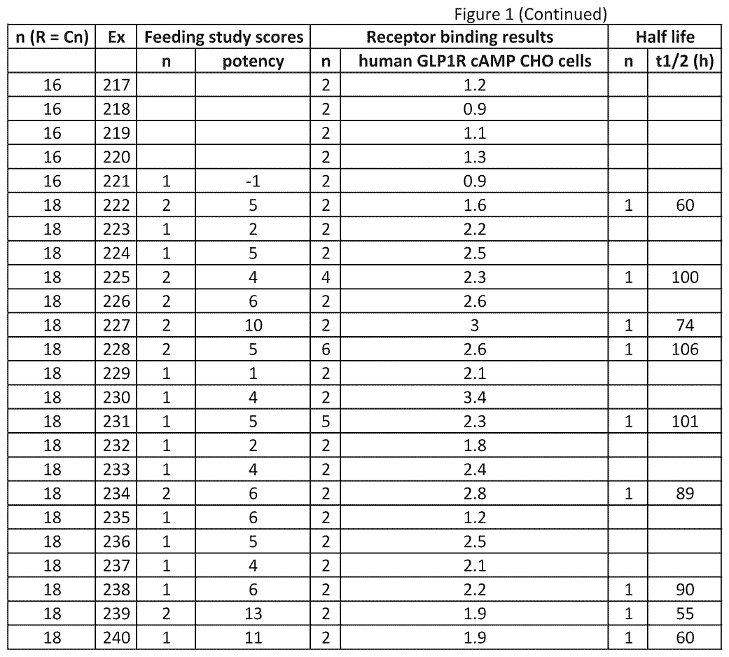

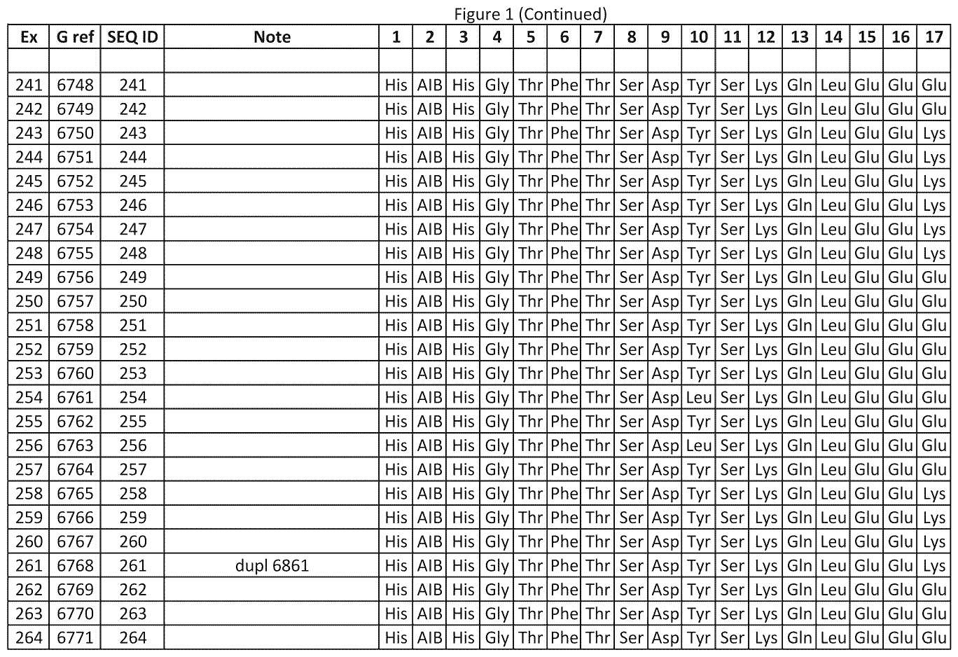

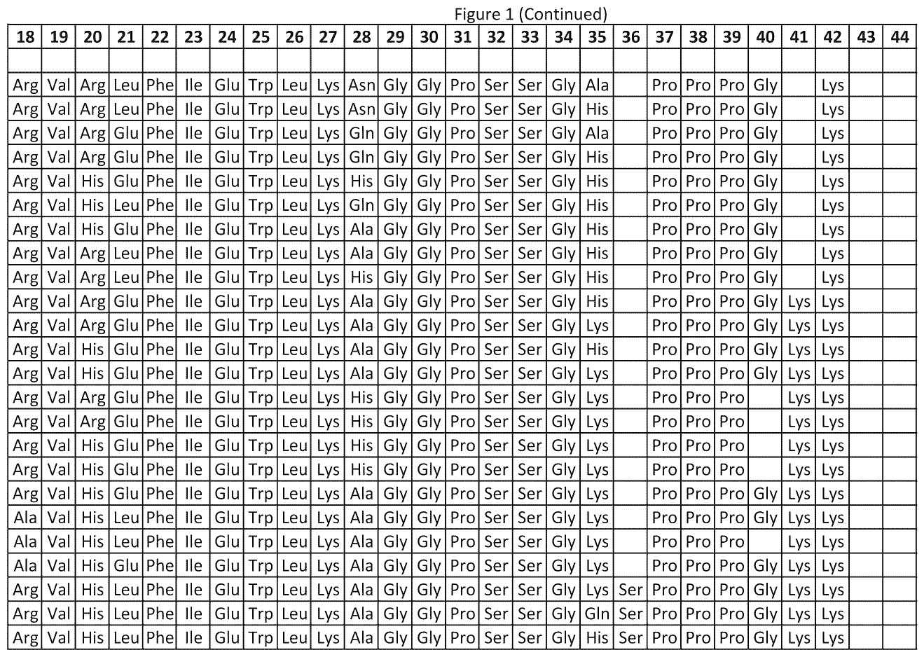

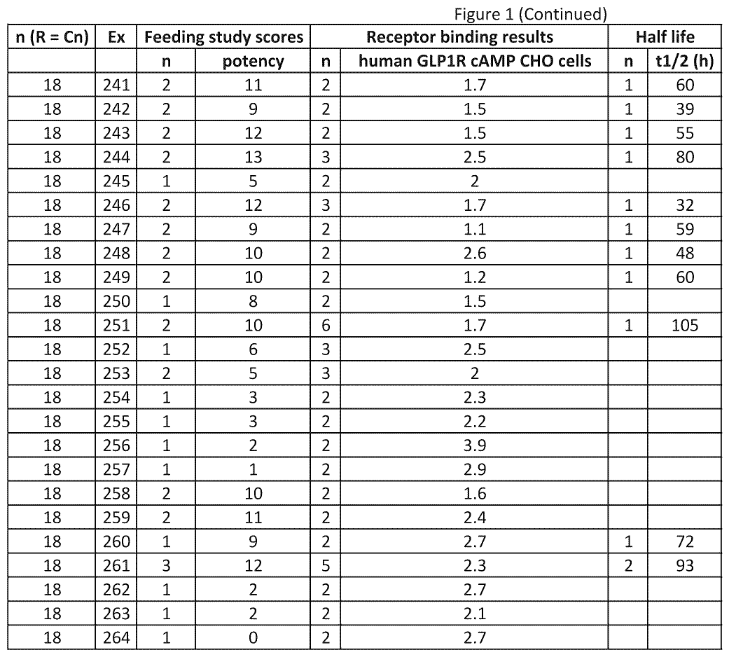

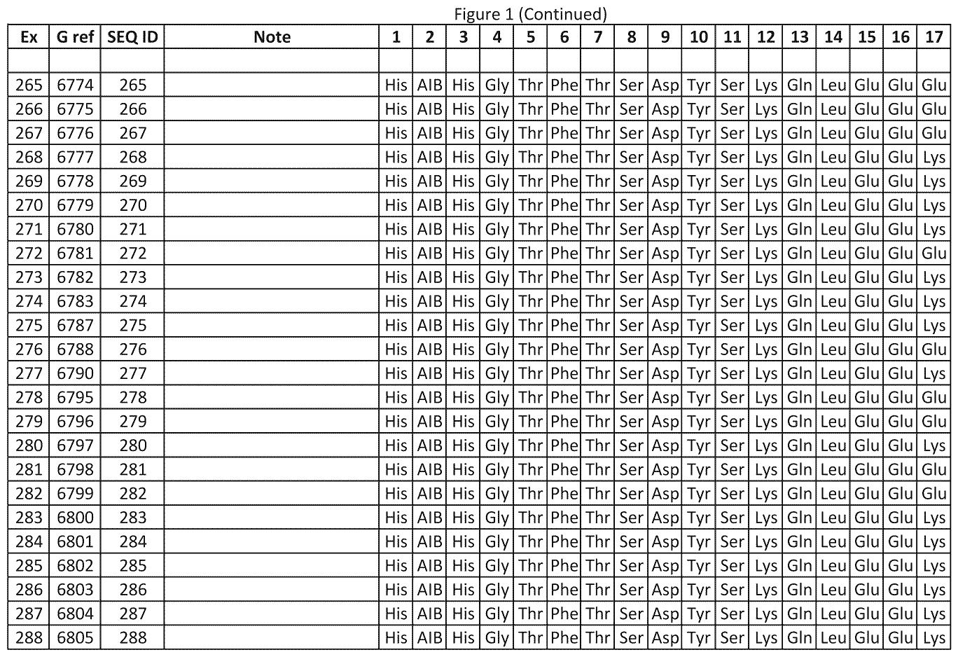

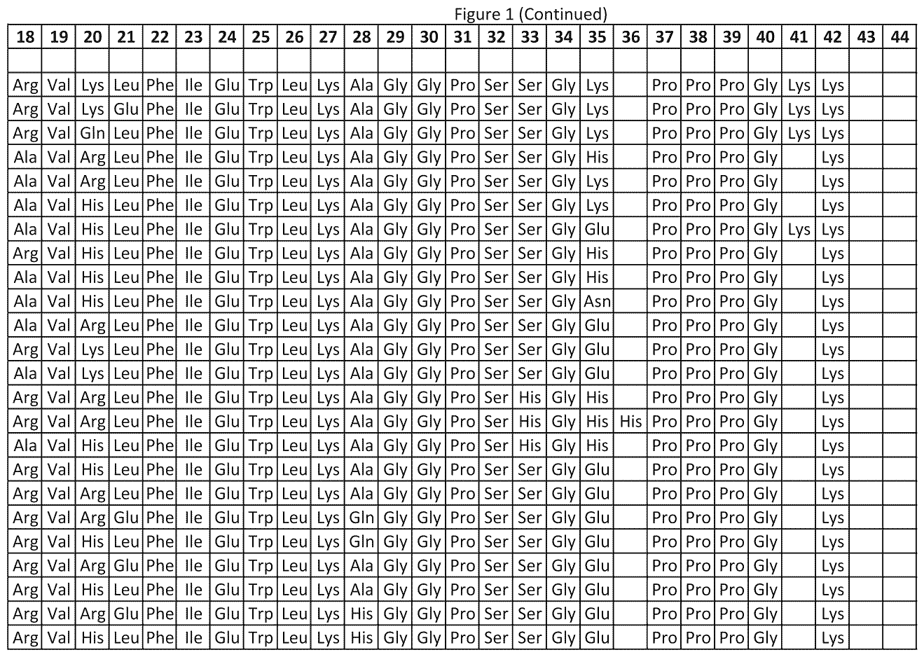

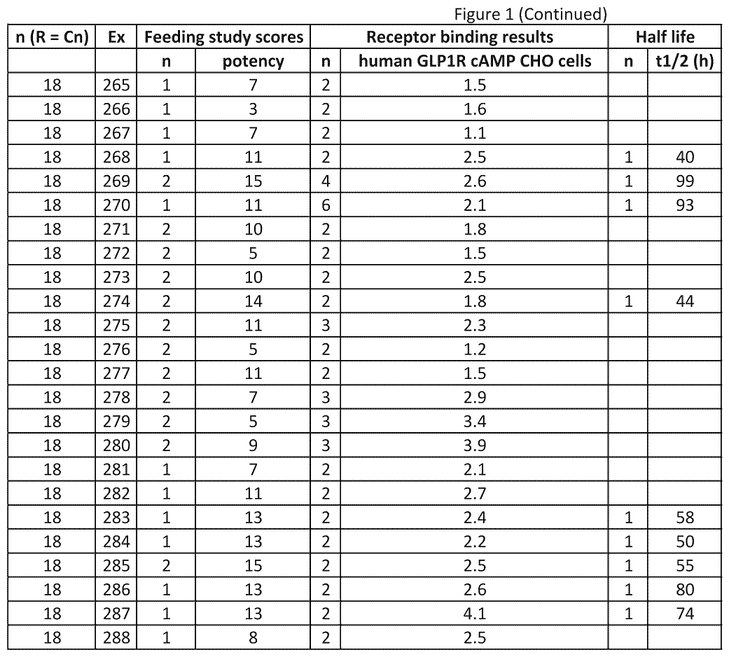

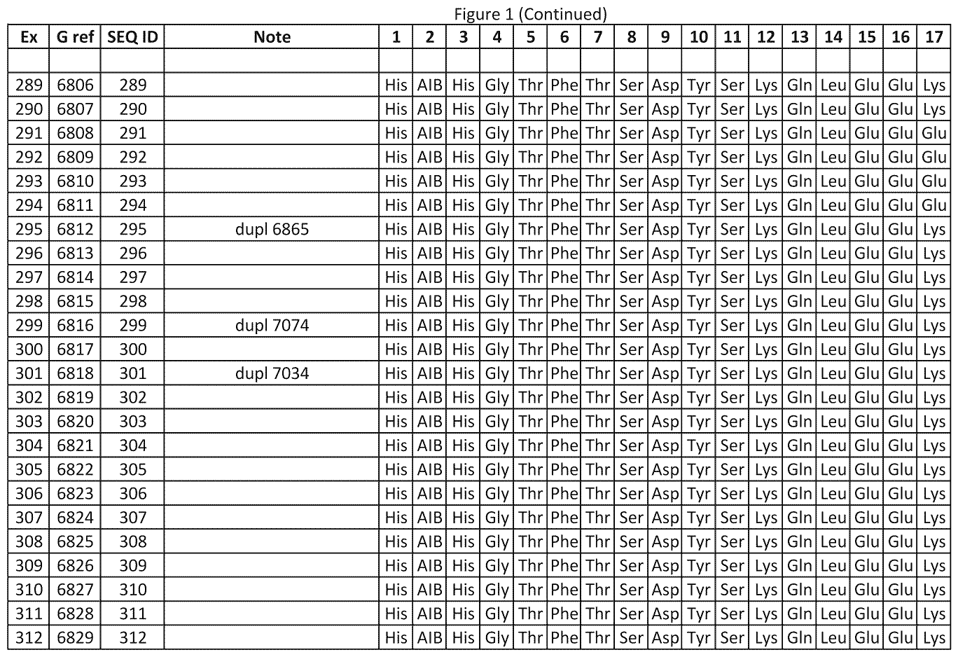

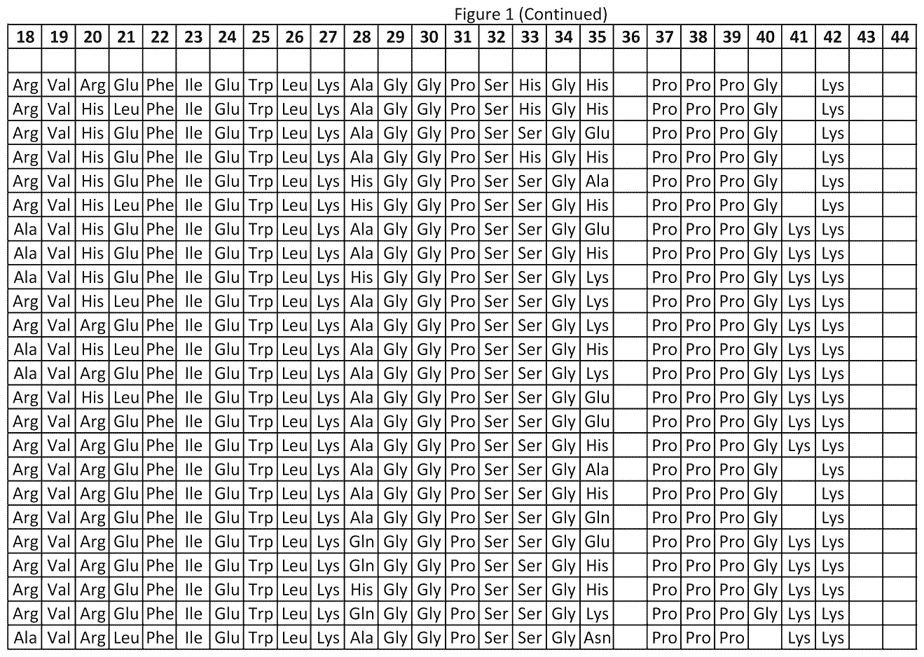

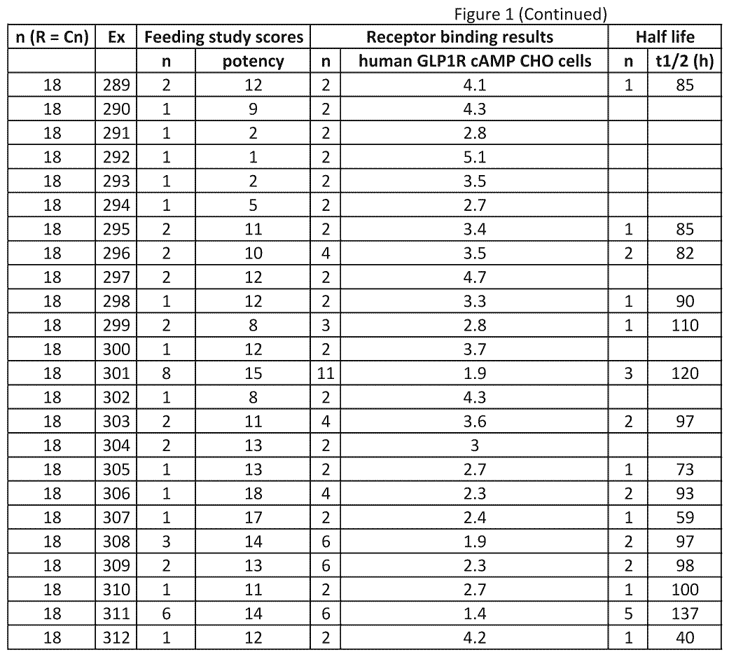

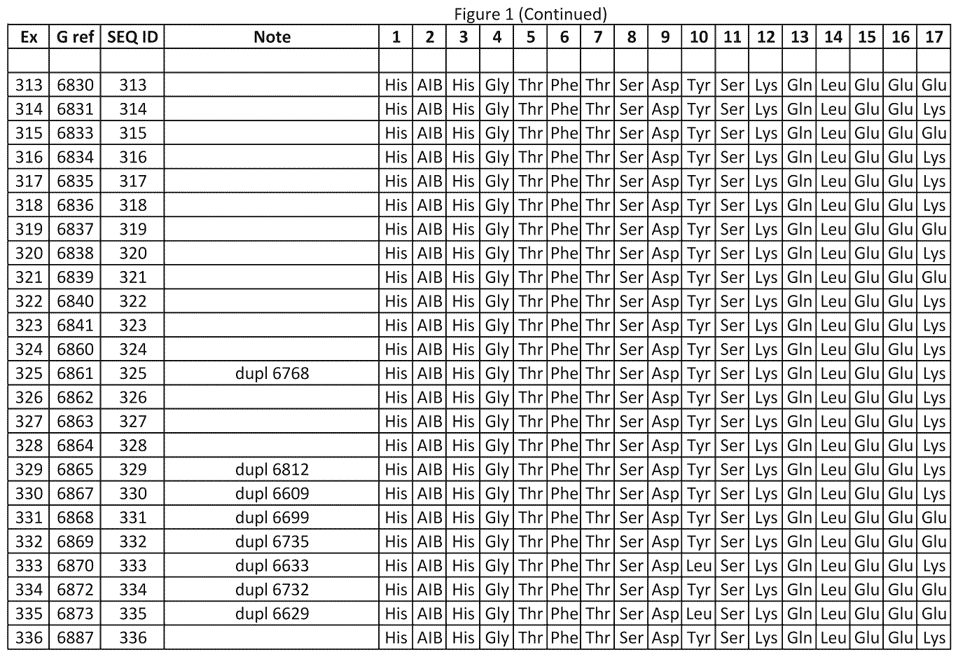

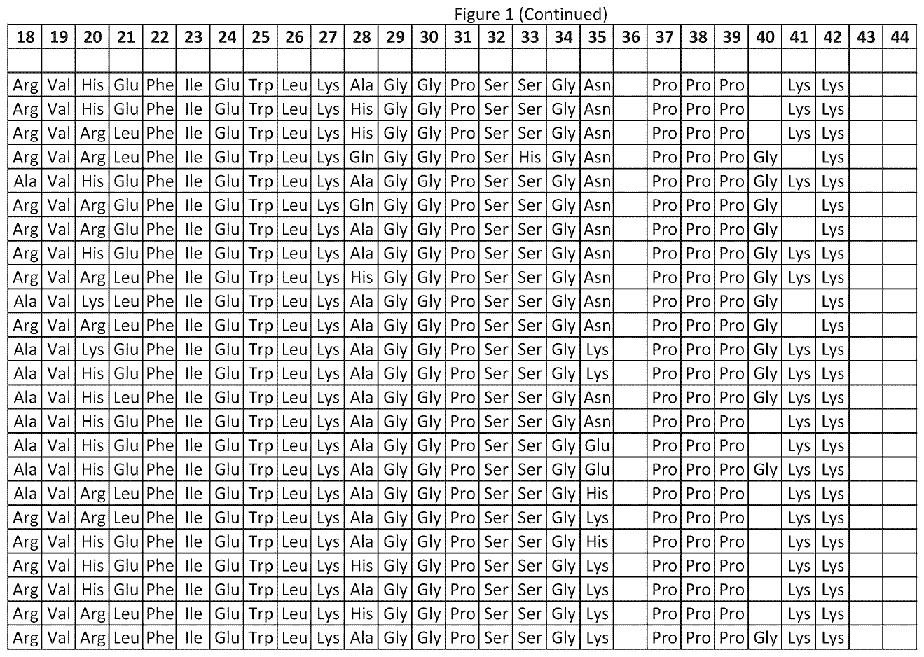

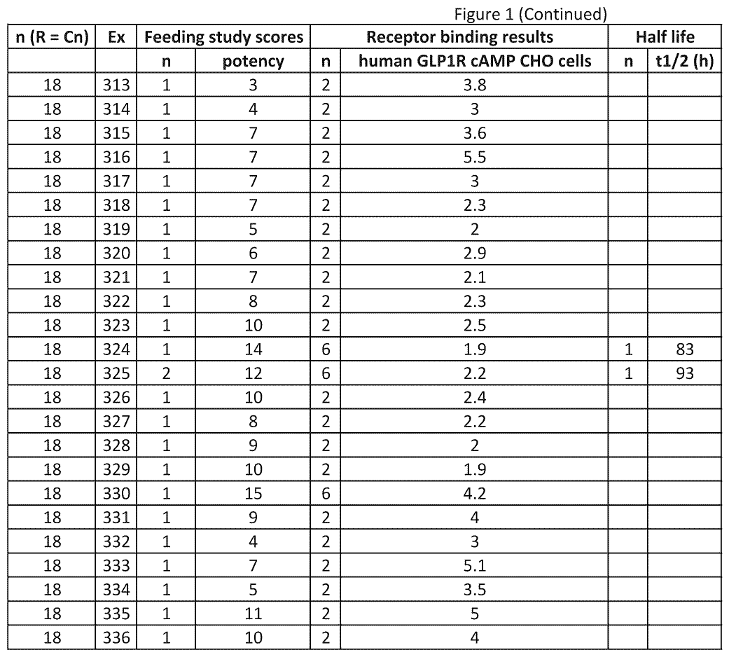

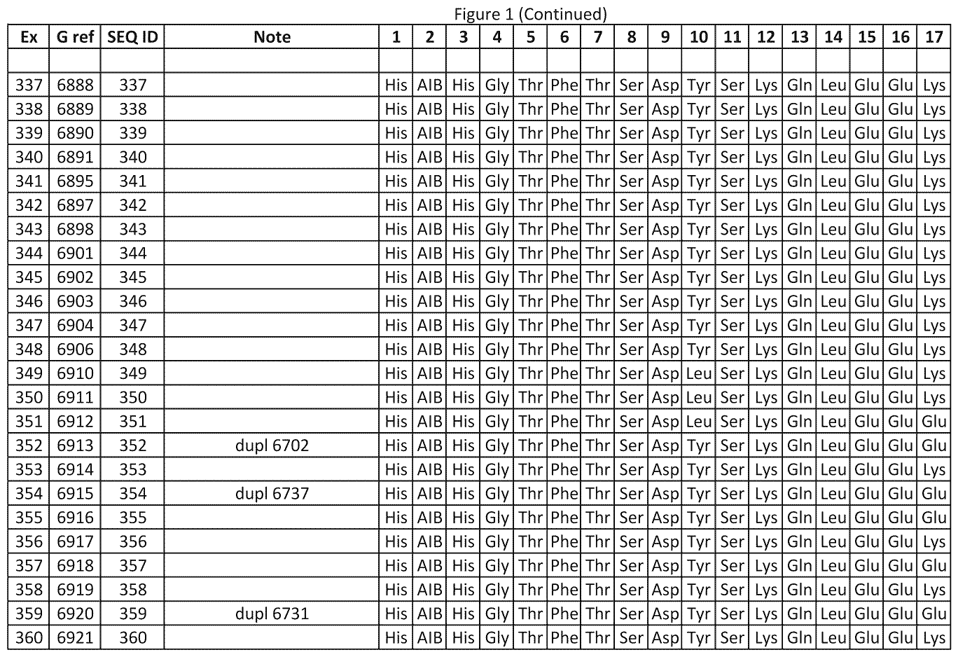

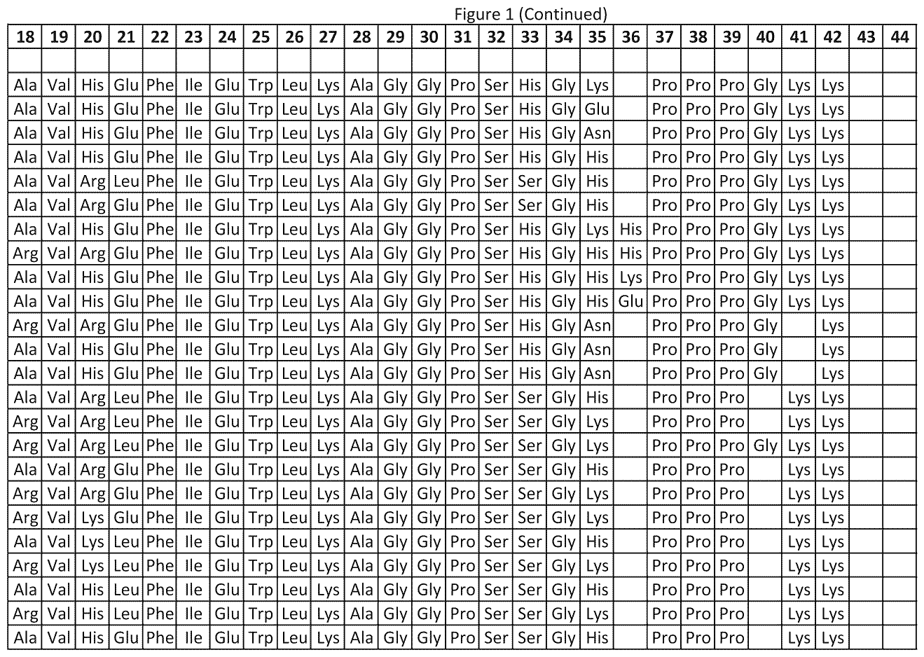

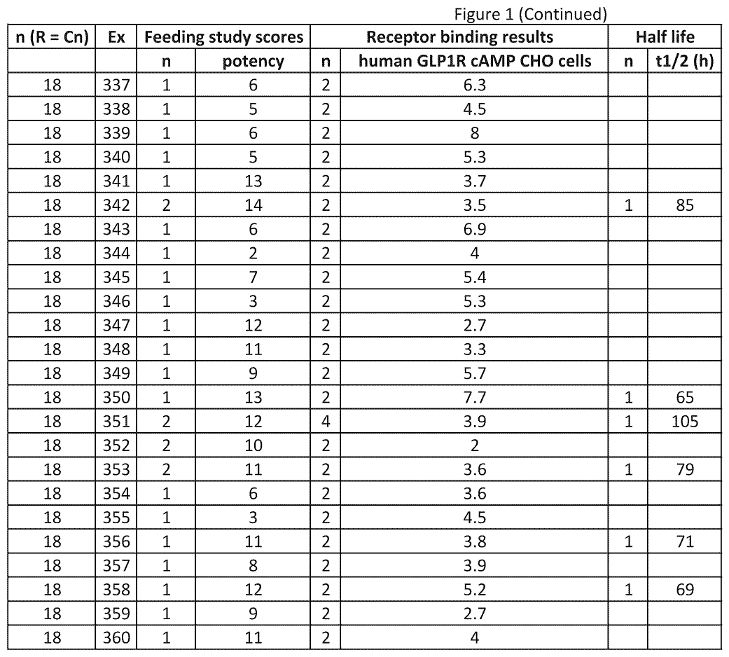

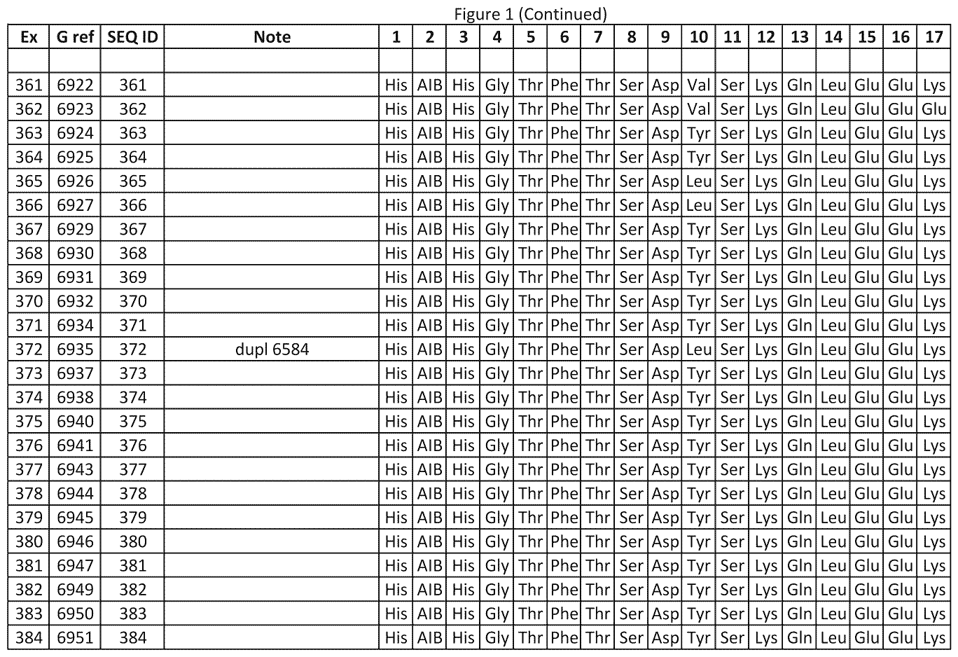

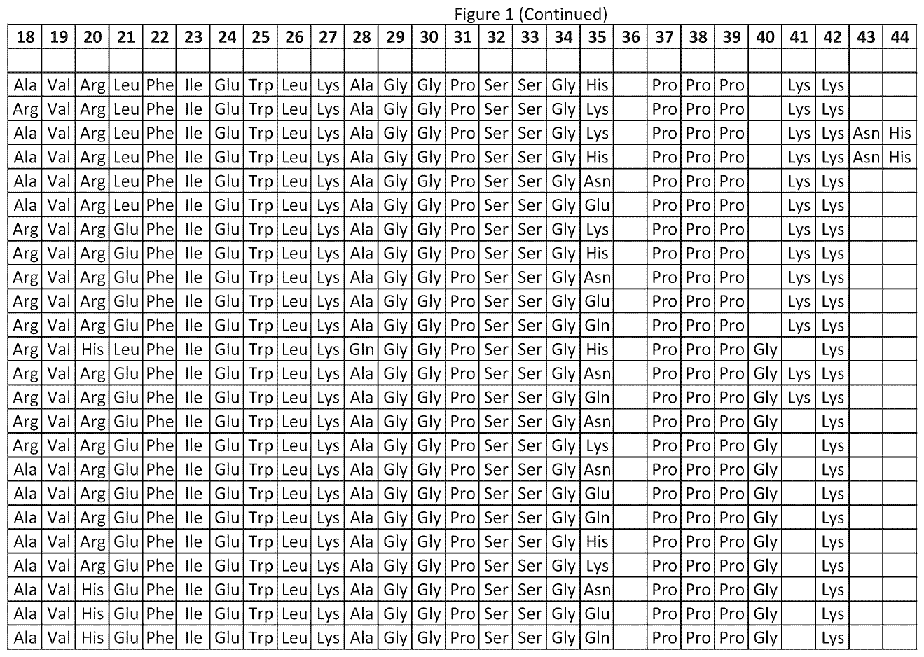

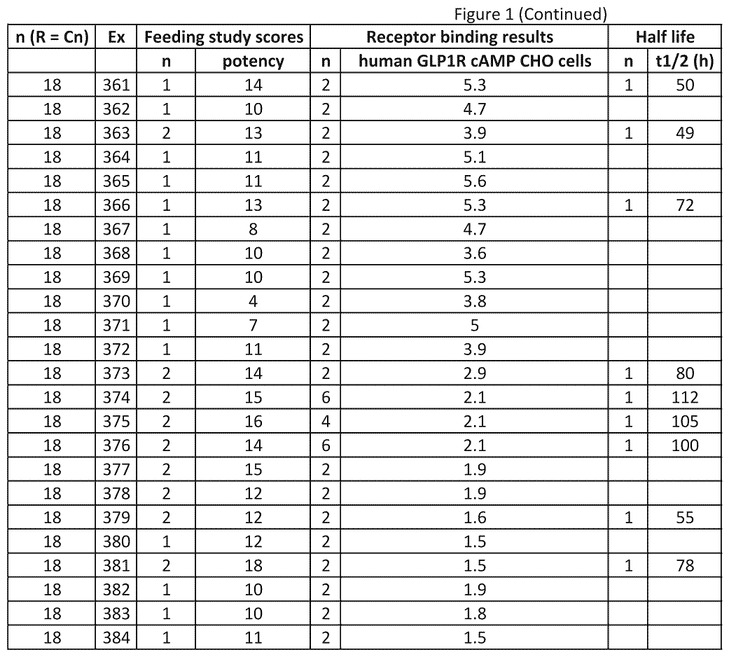

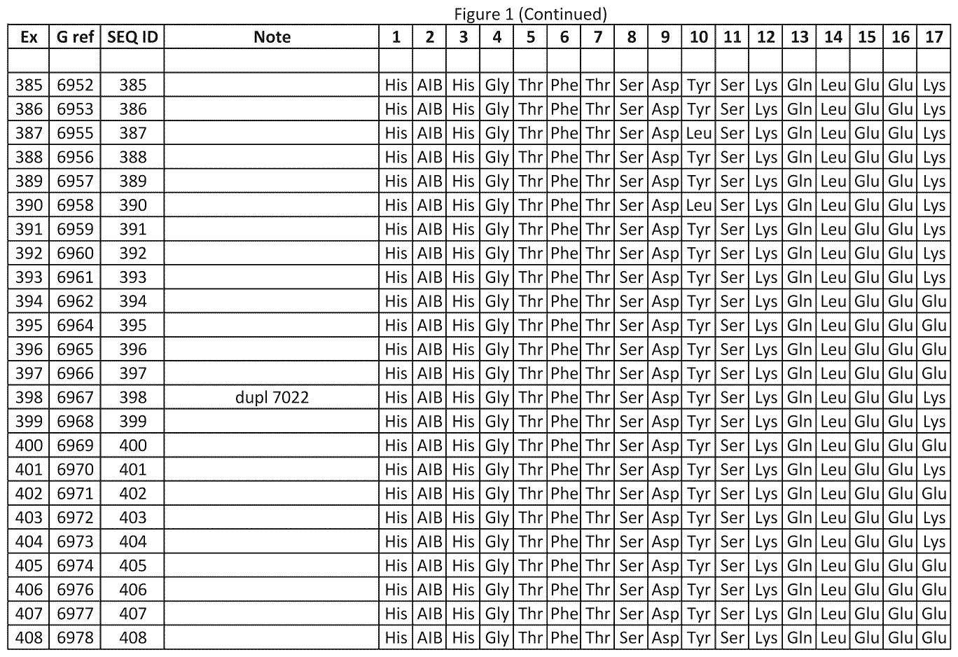

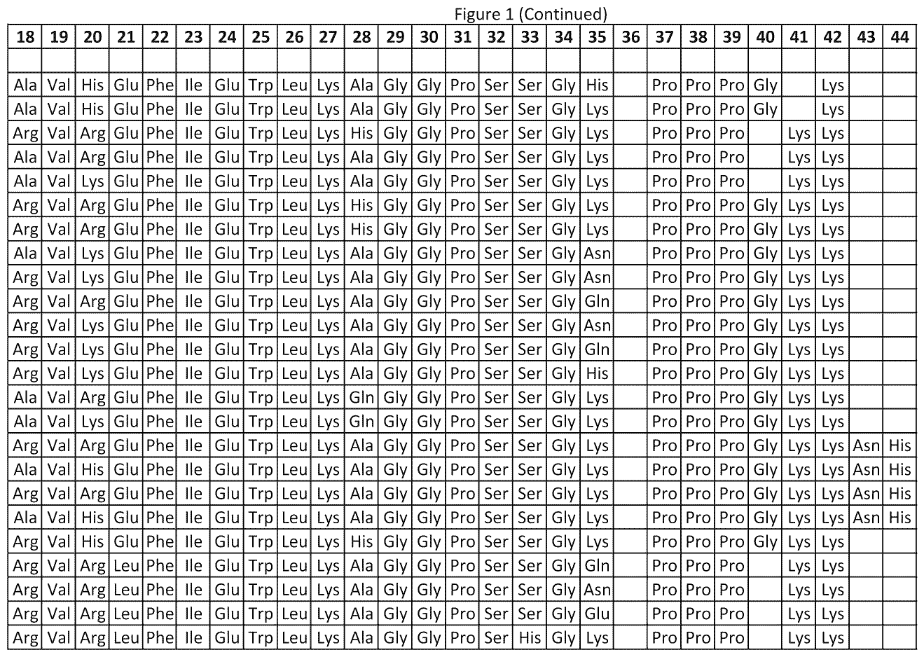

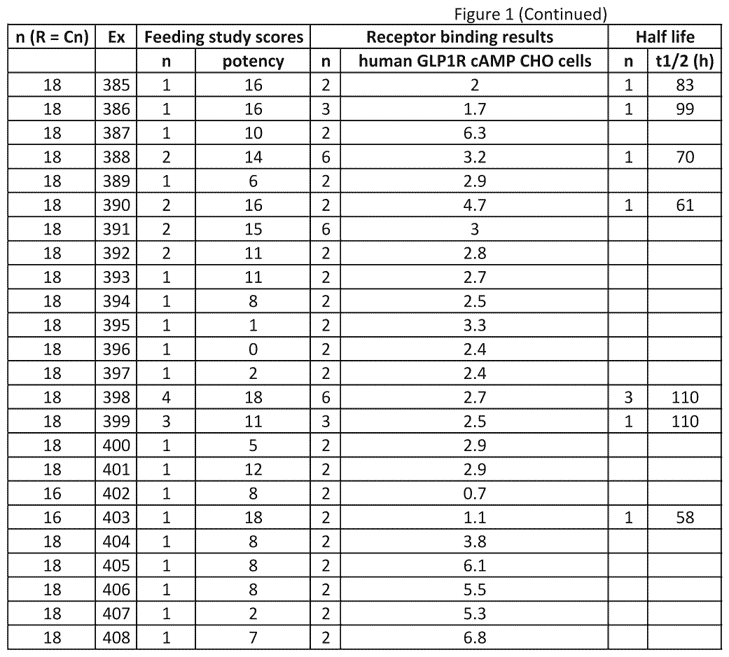

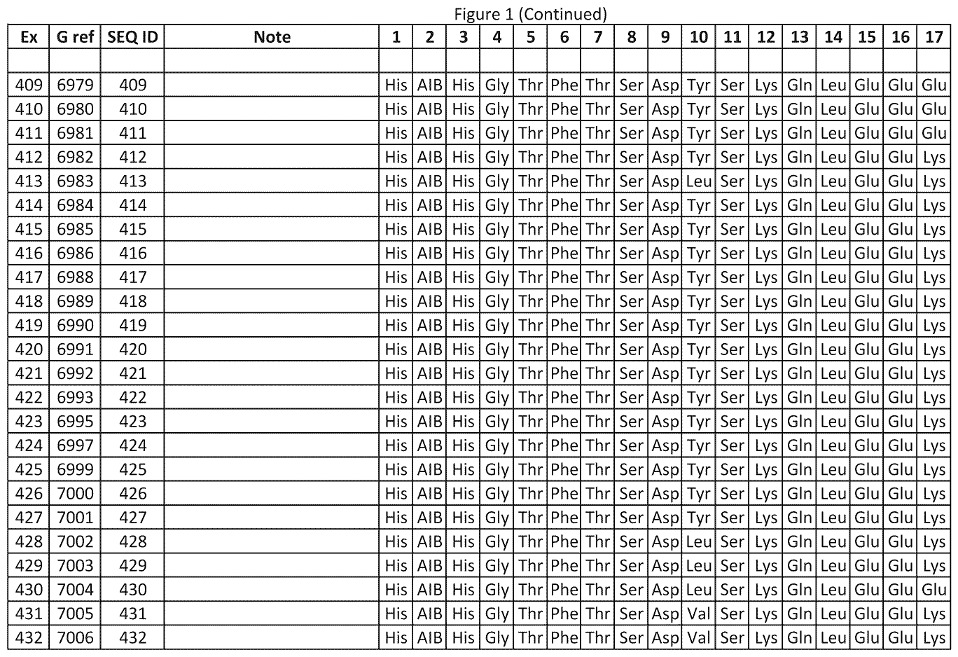

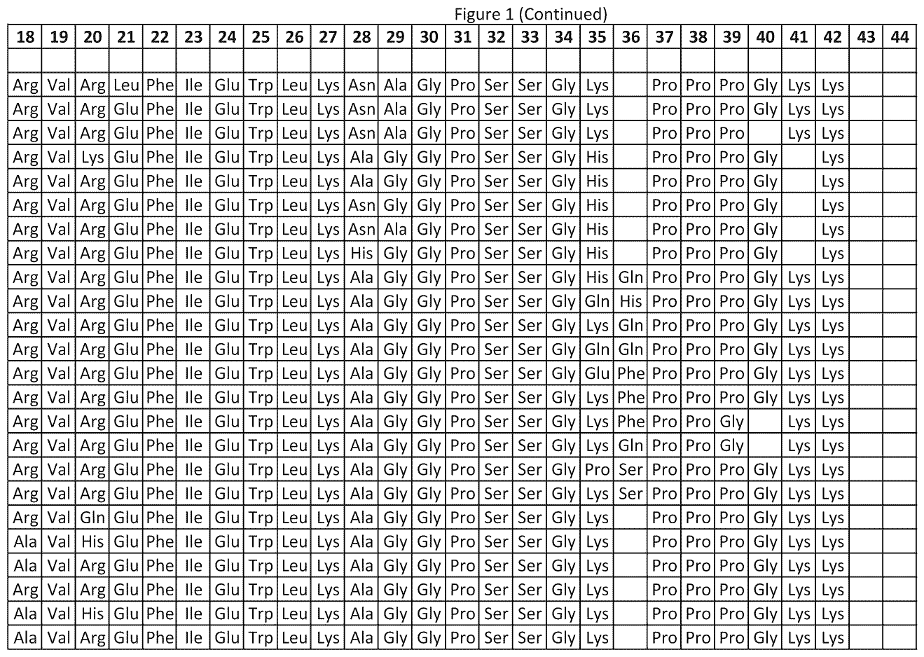

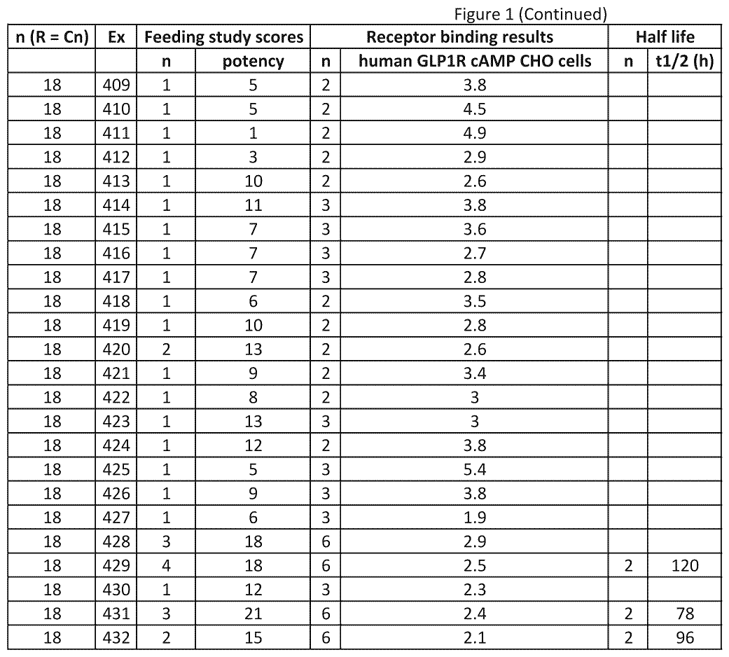

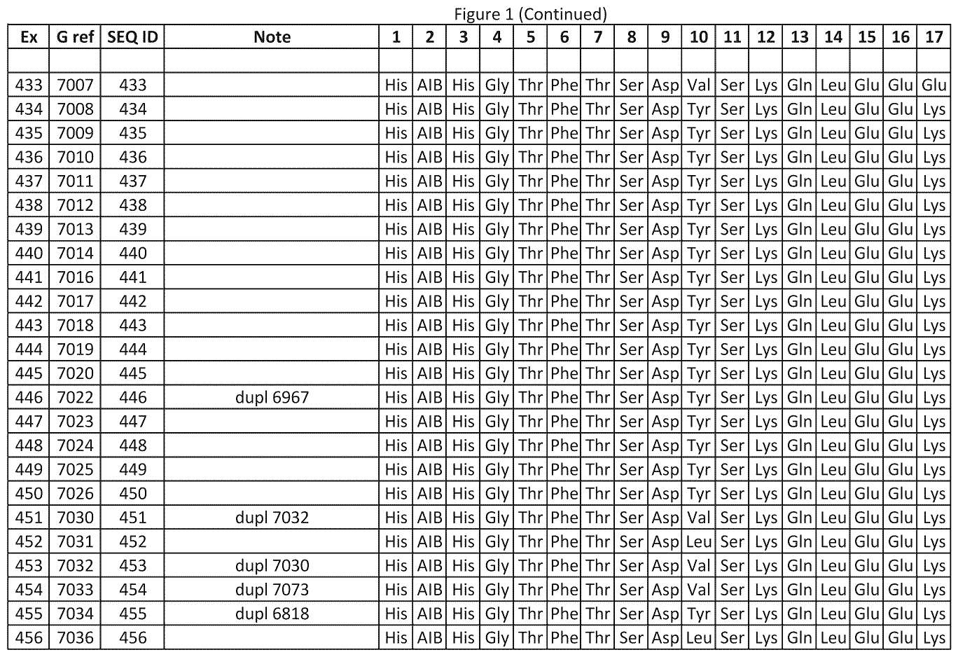

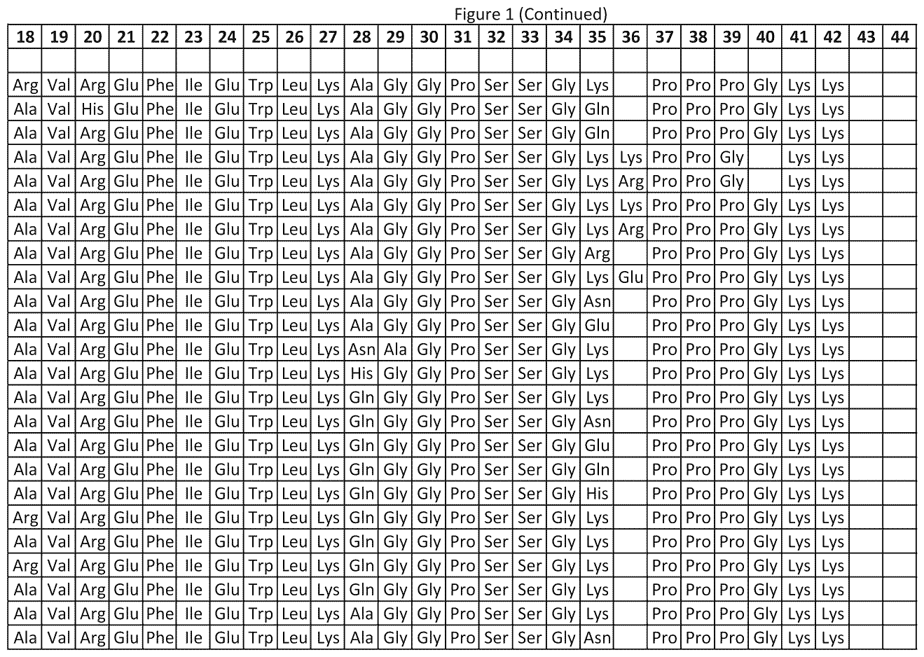

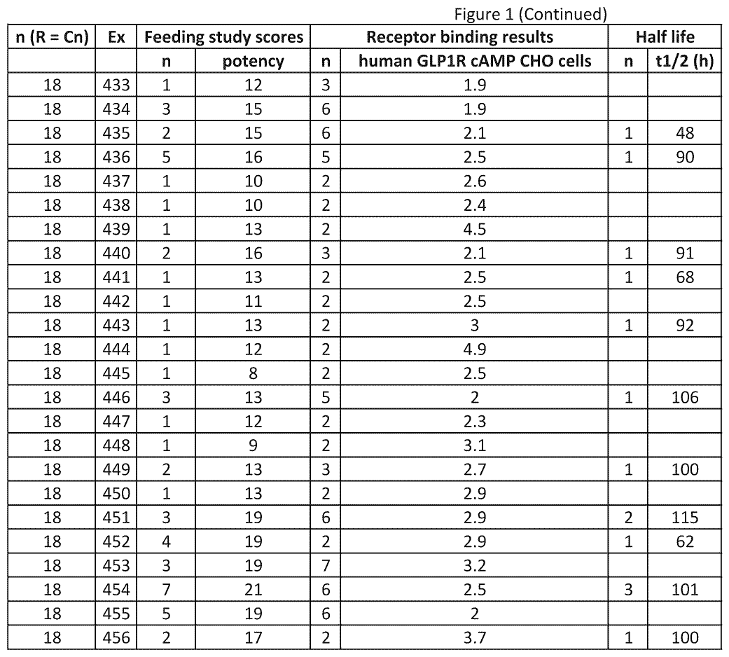

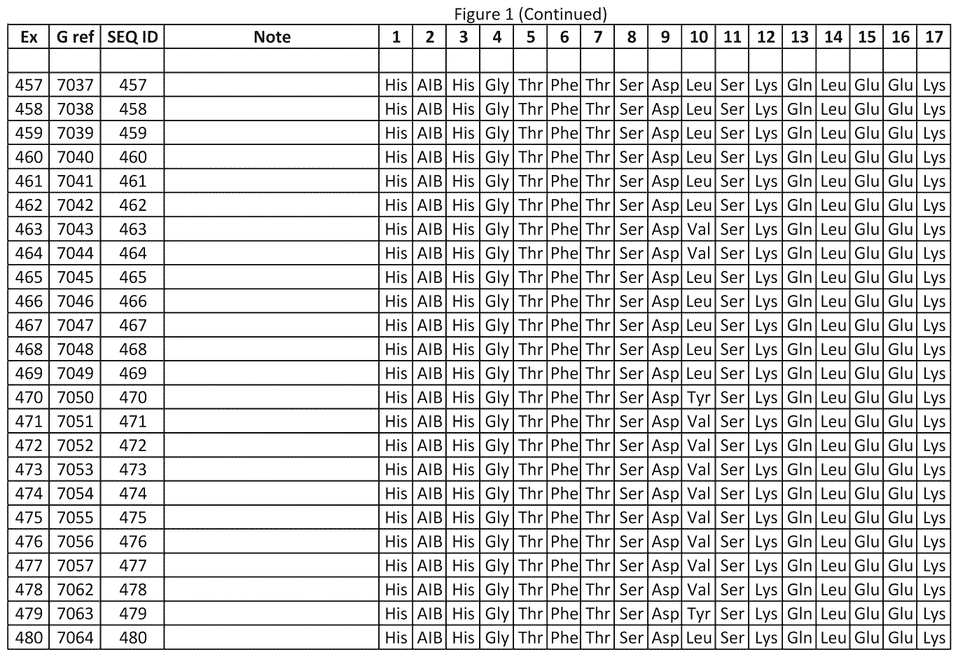

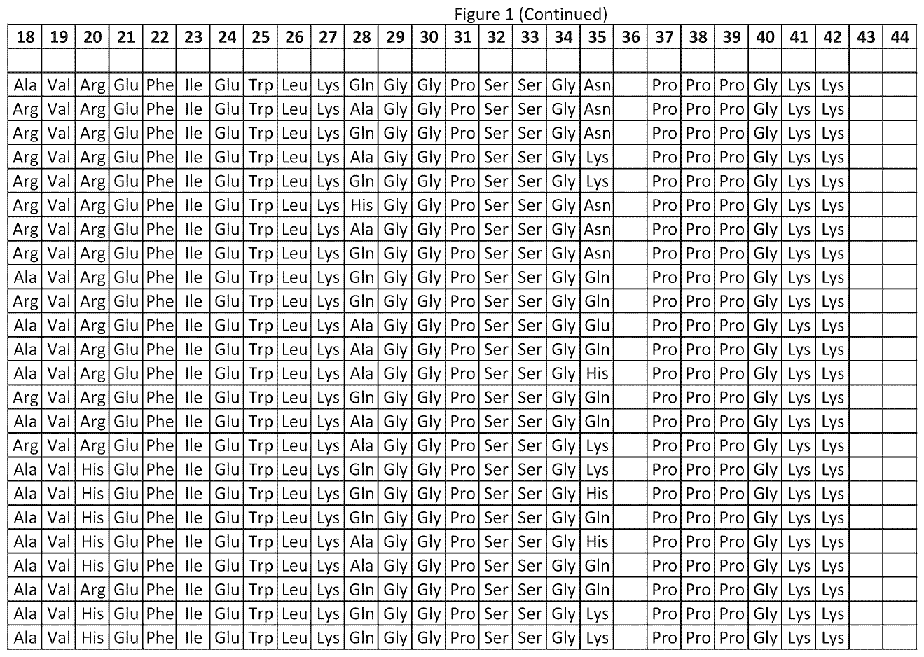

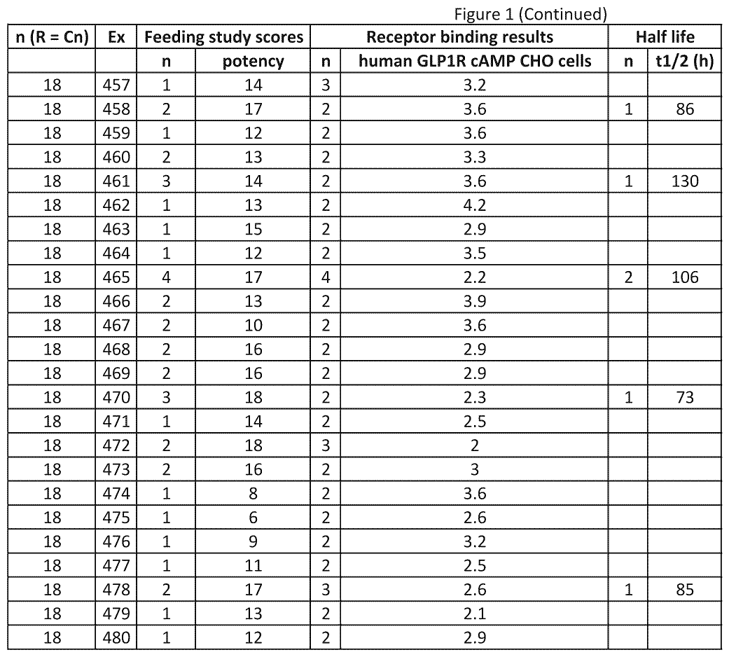

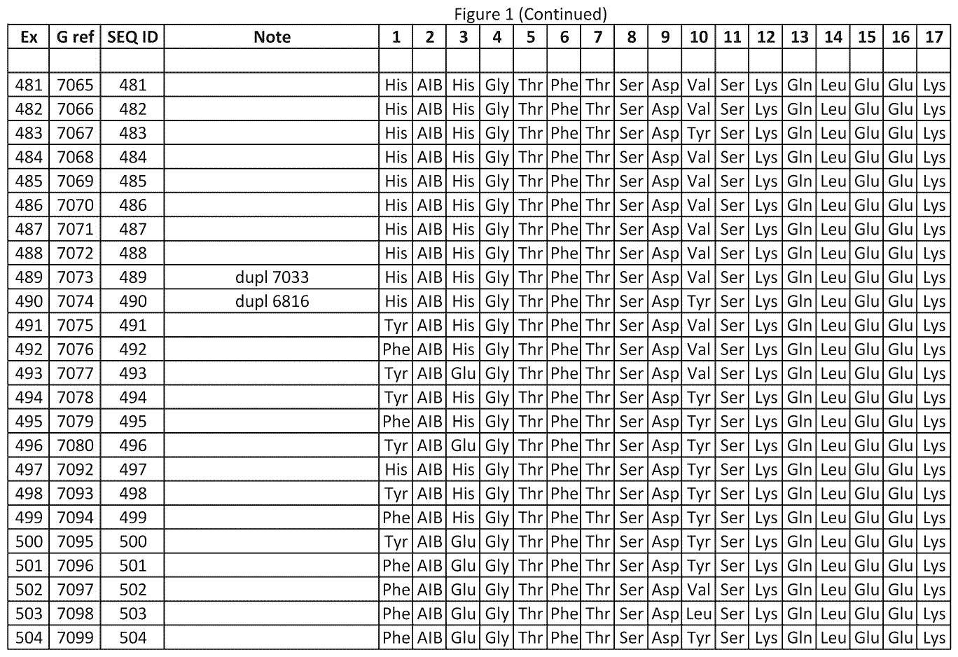

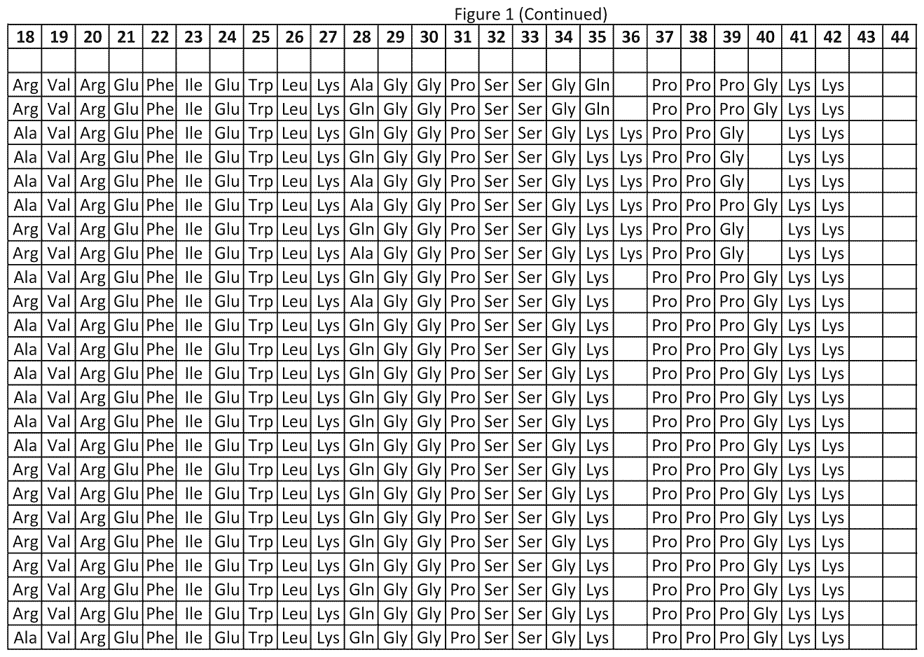

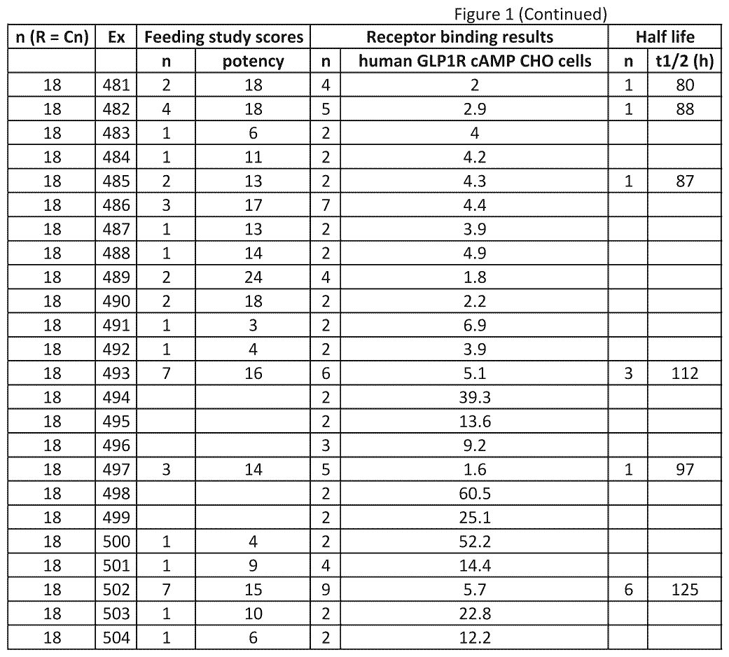

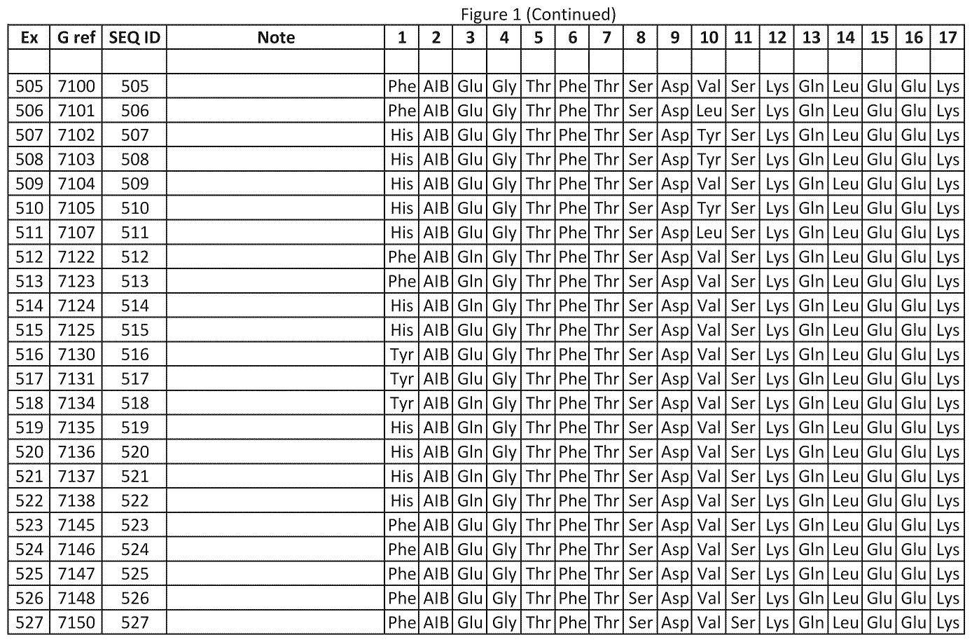

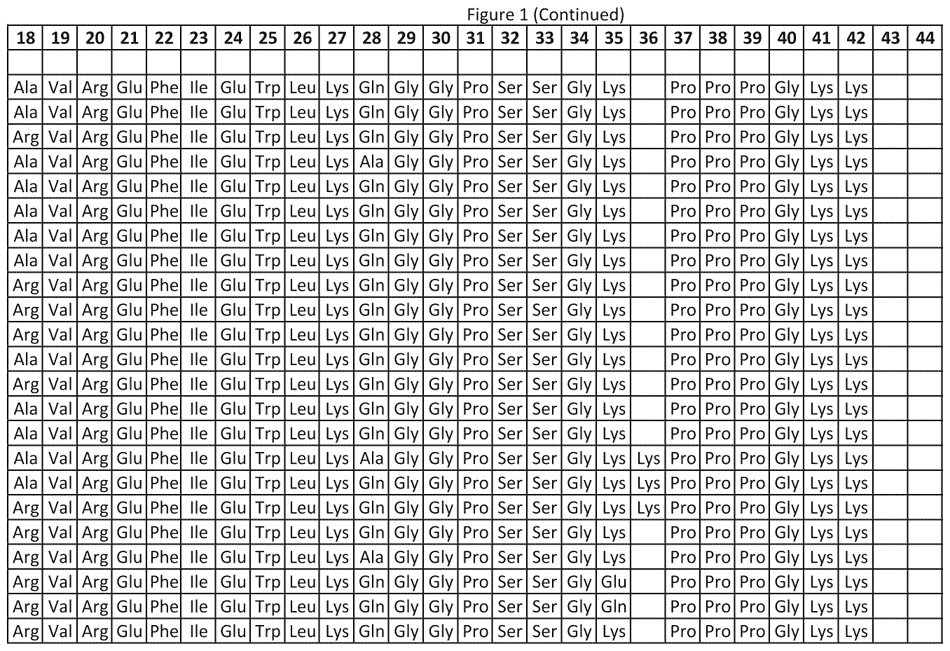

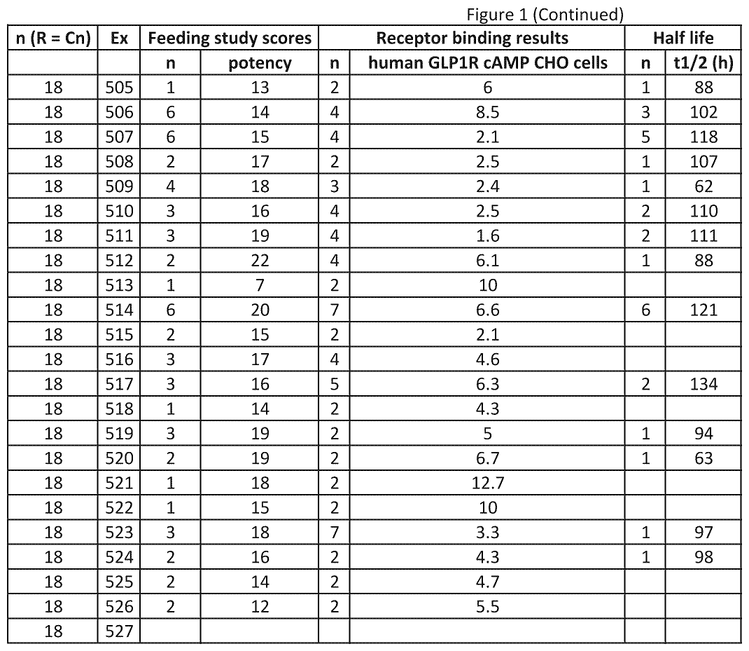

In especially preferred embodiments, the compound is one of the compounds of the invention set out in the Table of FIG. 1.

### Derivatives and Salts

The present invention provides compounds of formula (I), derivatives of such compounds, and salts or solvates of such compounds and derivatives.

The compounds, derivatives and salts may be produced by recombinant methods which are well-known in the art or alternatively they may be produced by synthetic methods, again which are well-known in the art.

### Derivatives

Whilst in some embodiments, the invention relates to a compound of formula (I) and is not a derivative, in other embodiments the invention relates to a derivative of a compound of formula (I). The derivative may for example comprise one or more derivatisations selected from amidation, glycosylation, carbamylation, acylation, sulfation, phosphorylation, cyclization, lipidization, pegylation and fusion to another peptide or protein to form a fusion protein, for example the derivative may comprise one or more derivatisations selected from amidation, glycosylation, carbamylation, acylation, sulfation, phosphorylation, cyclization, lipidization and pegylation. The structure may be modified at random positions within the molecule, or at predetermined positions within the molecule and may include one, two, three or more attached chemical moieties.

In certain embodiments it is preferred that the primary peptide chain of compounds of the invention may be amidated at their C-terminal. Such a modification is very common in nature with approximately half of naturally occurring peptides, including certain gastrointestinal peptide hormones, being susceptible to amidation at their C-terminal. The present invention encompasses all of the generic and specific sequences disclosed herein, including in the sequence listing and drawings, in both amidated and non-amidated forms, the amidation, where present being especially preferred on the C-terminal of the primary peptide sequence.

The derivative may for example be a fusion protein, whereby the structure of formula (I) is fused to another protein or polypeptide (the fusion partner) using recombinant methods known in the art. Alternatively, such a fusion protein may be synthetically synthesized by any known method. Such a fusion protein comprises the structure of formula (I). Any suitable peptide or protein can be used as the fusion partner (e.g., serum albumin, carbonic anhydrase, glutathione-S-transferase or thioredoxin, etc.). Such fusion proteins may be made by linking the carboxy-terminus of the fusion partner to the amino-terminus of the structure of formula (I) or vice versa. Optionally, a cleavable linker may be used to link the structure of formula (I) to the fusion partner. A resulting cleavable fusion protein may be cleaved in vivo such that an active form of a compound of the invention is released. Examples of such cleavable linkers include, but are not limited to, the linkers Asp-Asp-Asp-Asp-Tyr [SEQ ID NO: 535], Gly-Pro-Arg, Ala-Gly-Gly and His-Pro-Phe-His-Leu [SEQ ID NO: 536], which can be cleaved by enterokinase, thrombin, ubiquitin cleaving enzyme and renin, respectively. For details, see for example U.S. Pat. No. 6,410,707, the contents of which are incorporated herein by reference.

A derivative of the invention may for example be a physiologically functional derivative of the structure of formula (I). The term “physiologically functional derivative” is used herein to denote a chemical derivative of a compound of formula (I) having the same physiological function as the corresponding unmodified compound. For example, a physiologically functionally derivative may be convertible in the body to a compound of formula (I). According to the present invention, examples of physiologically functional derivatives include esters, amides, and carbamates; preferably esters and amides.

In addition to the derivatisation at Lys42, compounds of the invention can be further derivatised at additional positions. For example, pharmaceutically acceptable esters and amides of the compounds of the invention may comprise a C₁₋₂₀ alkyl-, C₂₋₂₀ alkenyl-, C₅₋₁₀-aryl-, C₅₋₁₀-ar-C₁-20 alkyl-, or amino acid- ester group or amide group attached at an appropriate site, for example formed by reaction of an alkyl, alkenyl aryl, aralkyl or amino alkyl group containing an alcohol or amino moiety with an acid moiety present in the compound of formula (I), or formed by reaction of an alkyl, alkenyl aryl, aralkyl or amino alkyl group containing an activated acyl group with an alcohol or amine group present in the compound of formula (I). Examples of suitable moieties are hydrophobic substituents with 4 to 26 carbon atoms, preferably 5 to 19 carbon atoms. Suitable lipid groups include fatty acids (e.g. lauroyl (C₁₂H₂₃), palmityl (C₁₅H₃₁), oleyl (C₁₅H₂₉) or stearyl (C₁₇H₃₅)) and bile acids (e.g. cholate or deoxycholate).

Methods for lipidization of sulfhydryl-containing compounds with fatty acid derivatives are disclosed in U.S. Pat. Nos. 5,936,092; 6,093,692; and 6,225,445, the contents of which are incorporated herein by reference. Fatty acid derivatives of a compound of the invention comprising a compound of the invention linked to fatty acid via a disulfide linkage may be used for delivery of a compound of the invention to neuronal cells and tissues. Lipidisation markedly increases the absorption of the compounds relative to the rate of absorption of the corresponding unlipidised compounds, as well as prolonging blood and tissue retention of the compounds. Moreover, the disulfide linkage in a lipidised derivative is relatively labile in the cells and thus facilitates intracellular release of the molecule from the fatty acid moieties. Suitable lipid-containing moieties are hydrophobic substituents with 4 to 26 carbon atoms, preferably 5 to 19 carbon atoms. Suitable lipid groups include fatty acids (e.g. lauroyl (C₁₂H₂₃), palmityl (C₁₅H₃₁), oleyl (C₁₅H₂₉) or stearyl (C₁₇H₃₅)) and bile acids (e.g. cholate or deoxycholate). Whilst lipid functionalised compounds of the invention may have benefits in certain situations, it is expected that in most cases, it will be simplest and preferred if a compound of the invention is not further derivatised, such that there are not additional lipid groups present.

Cyclization methods include cyclization through the formation of a disulfide bridge, and head-to-tail cyclization using a cyclization resin. Cyclized peptides may have enhanced stability, including increased resistance to enzymatic degradation, as a result of their conformational constraints. Cyclization may in particular be expedient where the uncyclized peptide includes an N-terminal cysteine group. Suitable cyclized peptides include monomeric and dimeric head-to-tail cyclized structures. Cyclized peptides may include one or more additional residues, especially an additional cysteine incorporated for the purpose of formation of a disulfide bond or a side chain incorporated for the purpose of resin-based cyclization.

The derivative may for example be a PEGylated structure of formula (I). Derivatives which are PEGylated compounds of the invention may provide additional advantages such as increased solubility, stability and circulating time of the polypeptide, or decreased immunogenicity (see U.S. Pat. No. 4,179,337, the contents of which are incorporated herein by reference).

Chemical moieties for derivatisation of a compound of the invention may also be selected from water soluble polymers such as polyethylene glycol, ethylene glycol/propylene glycol copolymers, carboxymethylcellulose, dextran, polyvinyl alcohol and the like. A polymer moiety for derivatisation of a compound of the invention may be of any molecular weight and may be branched or unbranched. For ease in handling and manufacturing, the preferred molecular weight of a polyethylene glycol for derivatisation of a compound of the invention is from about 1 kDa to about 100 kDa, the term “about” indicating that in preparations of polyethylene glycol, some molecules will weigh more, some less, than the stated molecular weight. Polymers of other molecular weights may be used, depending on the desired therapeutic profile, for example the duration of sustained release desired, the effects, if any, on biological activity, the ease in handling, the degree or lack of antigenicity and other known effects of the polyethylene glycol to a therapeutic protein or analog. For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa.

### Salts

Salt forms of compounds of formula (I) and of derivatives of such compounds also form part of the invention. In some embodiments the salt is a salt of a compound of formula (I). In other embodiments the salt is a salt of a derivative of a compound of formula (I).

Salts of compounds of the invention include those which are pharmaceutically acceptable, i.e. which are suitable for use in medicine. However, salts having non-pharmaceutically acceptable counterions are also within the scope of the present invention, for example, for use as intermediates in the preparation of the compounds.

Suitable salts according to the invention include those formed with organic or inorganic acids or bases. Pharmaceutically acceptable acid addition salts include those formed with hydrochloric, hydrobromic, sulphuric, nitric, citric, tartaric, acetic, phosphoric, lactic, pyruvic, acetic, trifluoroacetic, succinic, perchloric, fumaric, maleic, glycolic, salicylic, oxaloacetic, methanesulfonic, ethanesulfonic, p-toluenesulfonic, formic, benzoic, malonic, naphthalene-2-sulfonic, benzenesulfonic, and isethionic acids. Other acids such as oxalic acid may be useful as intermediates in obtaining the compounds of the invention in final form.

Pharmaceutically acceptable salts with bases include ammonium salts, alkali metal salts, for example potassium and sodium salts, alkaline earth metal salts, for example calcium and magnesium salts, and salts with organic bases, for example dicyclohexylamine and N-methyl-D-glucomine.

### Solvates

Those skilled in the art of organic and/or medicinal chemistry will appreciate that many organic compounds can form complexes with solvents in which they are reacted or from which they are precipitated or crystallized. Such complexes are known as “solvates”. For example, a complex with water is known as a “hydrate”. The invention also encompasses solvates of the compounds of formula (I), solvates of derivatives of the compounds, and solvates of salts of the derivatives.

Those skilled in the art of organic and/or medicinal chemistry will also appreciate than many organic compounds can exist in different forms, including as amorphous material and/or in one or more crystalline forms. Different physical forms of organic compounds are known as polymorphs. The invention also encompasses all such different physical forms of the compounds of formula (I), as well as different physical forms of their derivatives and salts.

### Biological Activity

Compounds of the invention have activity at the human GLP-1 receptor and can be considered GLP-1 receptor agonists. This may be assessed by, for example, an in vitro or cellular binding assay or by a reporter assay. Preferred compounds of the invention exhibit an activity at the human GLP-1 receptor that is at least 1/50^{th} that of human GLP-1, preferably an activity which is at least 1/30^{th}, 1/20^{th}, 1/10^{th}, ⅕^{th}, ⅓^{rd} or ½ that of human GLP-1, for example when tested in accordance with the assay described in the examples section below.

Methods of assessing activity at the GLP-1 receptor are well known. For example, Jones et al., Nat. Commun., 2018, 9(1), 1602 discloses a method of assaying for GLP-1 receptor binding. A specific method is described herein below.

Preferred compounds of the invention are effective in promoting insulin release/secretion. This may be assessed by, for example, an in vitro assay. Methods of assessing release of insulin from beta-cells are well known.

Compounds of the invention fulfil some, or more preferably all, of the following criteria:
1) Sustained bioactivity at the human GLP-1 receptor resulting in inhibition of appetite;
2) Activity in promoting insulin release from beta-cells;
3) High solubility in aqueous solution at pH 4.5 to allow an effective dose to be administered in a low volume injection (thereby resulting in lower pain of injection). Solubility may be easily assessed by simple in vitro tests;
4) Long period of activity in vivo (as assessed in humans or an animal model) so as to permit injections no more frequently than daily and preferably no more than twice, or more preferably no more than once a week, whilst still producing acceptable therapeutic or cosmetic benefits;
5) Good weight loss (as assessed in human subjects or an animal model);
6) Low antigenicity in humans. This may be assessed in humans or animal models (in particular mice which have been experimentally reconstituted with a human immune system so as to mimic human antibody repertoire) or predicted using predictive software such as that incorporating the “antigenic index” algorithm ((Jameson & Wolf (1988) Comput. Appl. Biosci. 4(1):181-6), or the PREDITOP algorithm (Pellequer & Westhof, (1993) J. Mol. Graph. 11(3):204-10), or using the methods of Kolaskar & Tongaonkar (1990) FEBS Lett. 10:276(1-2):172-4, the contents of which are incorporated herein by reference).

According to certain embodiments of the invention, especially embodiments relating to weight loss, obesity, carbohydrate metabolism and diabetes, the compounds, derivatives and salts of the invention have one, several or all of the following features:
A. Sufficient solubility between pH 4 and pH 5 to permit an effective dose to be administered in a volume of less than 1 ml, less than 0.5 ml or less than 0.3 ml;
B. Activation of cAMP signalling in Chinese hamster ovary cells over-expressing the human GLP-1 Receptor;
C. One, several or all of the further 1 to 6 features listed above.

### Pharmacokinetics, Duration of Action and Solubility

Compounds of the present invention exhibit potent and prolonged duration of action in vivo following subcutaneous administration. In order to achieve this, the compounds are required to have both good activity at the biological target, and excellent pharmacokinetic properties.

Compounds of the present invention have a therapeutically useful duration of action and that manifests itself in the beneficial effects being observed in the experiments described herein below over several days. The half-life of compounds of the invention was assessed in a pig PK model. Preferred compounds of the invention were found to have a half-life significantly longer than semaglutide. As well as exhibiting a long in vivo half-life, the compounds of the invention have good storage stability, with no significant degradation seen on storage in solution for 4 weeks at 4° C.

Poor water solubility is a known problem for lipid containing molecules. In contrast, the compounds of the invention have very good solubility.

Certain compounds of the invention include His residues. Incorporation of His residue(s) into peptides having poor aqueous solubility typically leads to peptides having enhanced solubility at acidic pH (e.g. pH 5) due to the presence of charged His side-chain groups, but which are less soluble at physiological pH (pH 7.4). The pI of the side-chain group of histidine is about 6.0. Such properties enable formulation of His-containing peptides in weakly acidic media. Upon subcutaneous injection of such formulations, the solubility falls leading to subcutaneous precipitation of peptide which resolubilises over time. Zinc-containing formulations of His-containing peptides enhance this effect, because at pH 7.4 but not at pH 5 zinc ions co-ordinate with histidine residues and result in a further reduction in solubility which can contribute to increased precipitation at a subcutaneous injection site, or which can contribute to increased stability of the precipitate. However, where precipitation of peptide is not sufficiently rapid following subcutaneous administration, there may still be an initial “spike” or “burst” in blood concentration levels of the peptide. Such properties are undesirable since they increase the possibility of subjects experiencing side effects associated with high concentration levels of the peptides, such as nausea, even if only temporary. In contrast to peptides not having the multi-His containing C-terminal sequence of the invention, the present compounds either do not display initial “spikes” or “bursts” in plasma concentration levels following subcutaneous administration or any such “burst” is significantly reduced. This reduces the likelihood and/or severity of possible side effects associated with high circulating levels of the compounds.

### Conditions

The invention also provides a compound, derivative or salt of the invention, or a composition comprising the compound, derivative or salt together with a pharmaceutically acceptable carrier and optionally a further therapeutic agent, for use as a medicament.

The invention also provides a method of treating or preventing a disease or disorder or other non-desired physiological state in a subject comprising administration of a therapeutically effective amount of a compound, derivative or salt of the invention, or of a composition comprising the compound, derivative or salt together with a pharmaceutically acceptable carrier and optionally a further therapeutic agent. Preferably the compound, derivative, salt or composition is administered subcutaneously.

According to certain embodiments, the disease or disorder or other non-desired physiological state is diabetes or obesity, and particularly diabetes (e.g. type II diabetes).

According to certain embodiments, the disease or disorder or other non-desired physiological state may be the physiological state of being overweight.

The subject to whom the compound is administered may be overweight, for example, obese. Alternatively, or in addition, the subject may be diabetic, for example having insulin resistance or glucose intolerance, or both. The subject may have diabetes mellitus, for example, the subject may have Type II diabetes. The subject may be overweight, for example, obese and have diabetes mellitus, for example, Type II diabetes.

In addition, or alternatively, the subject may have, or may be at risk of having, a disorder in which obesity or being overweight is a risk factor. Such disorders include, but are not limited to, heart disease, cardiovascular disease, for example hypertension, atherosclerosis, congestive heart failure, and dyslipidemia; stroke; gallbladder disease; osteoarthritis; sleep apnea; reproductive disorders for example, polycystic ovarian syndrome; cancers, for example breast, prostate, colon, endometrial, kidney, and esophagus cancer; varicose veins; acanthosis nigricans; eczema; exercise intolerance; insulin resistance; hypertension hypercholesterolemia; cholithiasis; osteoarthritis; orthopedic injury; insulin resistance, for example, type 2 diabetes and syndrome X; and thromboembolic disease (see Kopelman, Nature 404:635-43, 2000; Rissanen et al., British Med. J. 301, 835, 1990).

Other disorders associated with obesity include depression, anxiety, panic attacks, migraine headaches, PMS, chronic pain states, fibromyalgia, insomnia, impulsivity, obsessive compulsive disorder, and myoclonus. Certain neurological disorders and certain firms of neurological degeneration are also associated with obesity. Furthermore, obesity is a recognized risk factor for increased incidence of complications of general anesthesia (see e. g., Kopelman, Nature 404:635-43, 2000). In general, obesity reduces life span and carries a serious risk of co-morbidities such as those listed above.

Other diseases or disorders associated with obesity are birth defects, maternal obesity being associated with increased incidence of neural tube defects, carpal tunnel syndrome (CTS); chronic venous insufficiency (CVI); daytime sleepiness; deep vein thrombosis (DVT); end stage renal disease (ESRD); gout; heat disorders; impaired immune response; impaired respiratory function; infertility; liver disease; lower back pain; obstetric and gynecologic complications; pancreatitis; as well as abdominal hernias; acanthosis nigricans; endocrine abnormalities; chronic hypoxia and hypercapnia; dermatological effects; elephantitis; gastroesophageal reflux; heel spurs; lower extremity edema; mammegaly which causes considerable problems such as bra strap pain, skin damage, cervical pain, chronic odours and infections in the skin folds under the breasts, etc.; large anterior abdominal wall masses, for example abdominal panniculitis with frequent panniculitis, impeding walking, causing frequent infections, odours, clothing difficulties, low back pain; musculoskeletal disease; pseudotumor cerebri (or benign intracranial hypertension), and sliding hiatal hernia.

In some embodiments, the disease or disorder may be non-alcoholic fatty liver disease.

According to certain embodiments the disease or disorder or other non-desired physiological state may be being of a non-desired weight despite not being obese or overweight. The subject may be of normal weight (this includes but is not limited to subjects who were previously overweight or obese and who wish to prevent a return to an unhealthy weight). A subject may be a subject who desires weight loss, for example female and/or male subjects who desire a change in their appearance. In some cases where the subject is of a normal weight, aspects of the invention may relate to cosmetic treatment rather than to therapeutic treatment.

The invention also provides a method of reducing appetite in a subject, reducing food intake in a subject, reducing calorie intake in a subject, improving insulin release in a subject, improving carbohydrate metabolism in a subject, and/or improving carbohydrate tolerance in a subject, comprising administration of a therapeutically effective amount of a compound, derivative, salt or composition of the invention. Such methods may relate to treating subjects having a pre-diabetic state such as insulin insensitivity or pre-diabetes.

The invention also provides a method for improving a lipid profile in a subject comprising administration of a therapeutically effective amount of a compound, derivative, salt or composition of the invention. The invention also provides a method for alleviating a condition or disorder that can be alleviated by reducing nutrient availability comprising administration of a therapeutically effective amount of a compound, derivative, salt or composition of the invention.

A compound, derivative, salt or composition of the invention may be used for weight control and treatment, for example reduction or prevention of obesity, in particular any one or more of the following: preventing and reducing weight gain; inducing and promoting weight loss; and reducing obesity as measured by the Body Mass Index. A compound, derivative, salt or composition of the invention may be used in maintaining any one or more of a desired body weight, a desired Body Mass Index, a desired appearance and good health.

The present invention may also be used in treating, prevention, ameliorating or alleviating conditions or disorders caused by, complicated by, or aggravated by a relatively high nutrient availability. The term “condition or disorder which can be alleviated by reducing caloric (or nutrient) availability” is used herein to denote any condition or disorder in a subject that is either caused by, complicated by, or aggravated by a relatively high nutrient availability, or that can be alleviated by reducing nutrient availability, for example by decreasing food intake. Subjects who are insulin resistant, glucose intolerant, or have any form of diabetes mellitus, for example, type 1, 2 or gestational diabetes, can also benefit from methods in accordance with the present invention.

Conditions or disorders associated with increased caloric intake include, but are not limited to, insulin resistance, glucose intolerance, obesity, diabetes, including type 2 diabetes, eating disorders, insulin-resistance syndromes, and Alzheimer's disease. J. Cereb. Blood Flow Metab. 2011 Apr. 13 (Teramoto S et al.) discusses the use of both GLP-1 and exendin-4 to confer cardioprotection after myocardial infarction and demonstrates that exendin-4 may be used to provide neuroprotection against cerebral ischemia-reperfusion injury. The study showed that mice receiving a transvenous injection of exendin-4, after a 60-minute focal cerebral ischemia showed significantly reduced infarct volume and improved functional deficit as well as suppressed oxidative stress, inflammatory response, and cell death after reperfusion. The study provided evidence that the protective effect of exendin-4 is mediated through increased intracellular cAMP levels and suggested that exendin-4 is potentially useful in the treatment of acute ischemic stroke.

Accordingly, the invention also provides a method of providing cytoprotection in a subject, such as providing cardiac protection, providing neuroprotection and/or treating or preventing neurodegeneration, comprising administration of a therapeutically effective amount of a compound, derivative, salt or composition of the invention.

In certain embodiments the disease or disorder or other non-desired physiological state which the compound, derivative, salt or composition of the invention may be used to treat or prevent is neurodegeneration. Such neurodegeneration may be caused by apoptosis, necrosis or loss of function of neuronal cells, preferably in the CNS. Neurodegeneration treated or prevented may be that following a brain injury (for example following physical trauma or following a non-traumatic injury such a stroke, tumor, hypoxia, poisoning, infection, ischemia, encephalopathy or substance abuse). Alternatively or additionally, neurodegeneration may be prevented or treated in a subject having (or diagnosed as having a predisposition to) a neurodegenerative disease such as Alzheimer's disease, Parkinson's disease, Gehrig's disease (Amyotrophic Lateral Sclerosis), Huntington's disease, Multiple Sclerosis, other demyelination related disorders, senile dementia, subcortical dementia, arteriosclerotic dementia, AIDS-associated dementia, other dementias, cerebral vasculitis, epilepsy, Tourette's syndrome, Guillain Barre Syndrome, Wilson's disease, Pick's disease, neuroinflammatory disorders, encephalitis, encephalomyelitis, meningitis, other central nervous system infections, prion diseases, cerebellar ataxias, cerebellar degeneration, spinocerebellar degeneration syndromes, Friedrich's ataxia, ataxia teangiectasia, spinal dysmyotrophy, progressive supranuclear palsy, dystonia, muscle spasticity, tremor, retinitis pigmentosa, striatonigral degeneration, mitochondrial encephalomyopathies, neuronal ceroid lipofuscinosis. Preferably, the neurodegenerative disease is selected from the group consisting of Alzheimer's disease, Parkinson's disease, Gehrig's disease (Amyotrophic Lateral Sclerosis) and Huntington's disease. In such circumstances the treatment would be regarded as neuroprotective. According to certain preferred embodiments, the treatment is neuroprotective following cerebral ischemia or neuroprotective in a subject having a neurodegenerative disease or diagnosed as having a predisposition to a neurodegenerative disease.

According to other embodiments the disease or disorder or other non-desired physiological state is cardiac degeneration (in particular myocardial degeneration by apoptosis, necrosis or loss of function of myocardial cells), in which case the compound, derivative, salt or composition according to the invention provides cardiac protection. According to certain preferred embodiments that treatment is protective of myocardial function following myocardiac infarction.

The invention also provides a compound, derivative, salt or composition of the invention, for use in the treatment of obesity or diabetes.

The invention also provides a compound, derivative, salt or composition of the invention, for use in increasing energy expenditure of a subject, improving insulin release in a subject, improving carbohydrate tolerance in a subject and/or improving carbohydrate metabolism in a subject. Such use may relate to treating subjects having a pre-diabetic state such as insulin insensitivity or pre-diabetes.

The invention also provides a compound, derivative, salt or composition of the invention, for use in the reduction of appetite in a subject, use in the reduction of food intake in a subject, use in the reduction of calorie intake in a subject, use in improving insulin release in a subject, and/or use in improving carbohydrate tolerance in a subject. Such use may relate to treating subjects having a pre-diabetic state such as insulin insensitivity or pre-diabetes.

The invention also provides a compound, derivative, salt or composition of the invention, for use as a cytoprotective agent (e.g. in treating or preventing neurodegeneration, providing neuroprotection and/or providing cardiac protection). For example, the compound, derivative, salt or composition may be for use in myocardial protection in a subject following myocardial infarction, or for use in neuroprotection in a subject following cerebral ischemia or stroke, or for use in neuroprotection in a subject having a chronic neurodegenerative disease. Various features of neuroprotective or cardioprotective use of the compound, derivative, salt or composition may be as outlined above in relation to methods of the invention.

In the case of neuroprotection, the subject may have experienced previously a brain injury, stroke or other event causing cerebral ischemia. Alternatively, the subject may have or have been diagnosed with a predisposition to develop a chronic neurodegenerative disease. In the case of cardioprotection the subject may have experienced previously an event causing myocardial ischemia such as a myocardial infarction and angina. According to some embodiments a compound, derivative, salt or composition of the invention may be administered as soon as possible after the subject has experienced a suspected myocardial infarction. According to certain embodiments a compound, derivative, salt or composition of the invention may be administered as soon as possible after the subject has experienced as suspected stroke.

The invention also provides use of a compound, derivative, salt or composition of the invention for the manufacture of a medicament for the treatment of obesity or diabetes, of a subject, who may be as described above in reference to other aspects of the invention.

The invention also provides use of a compound, derivative or salt of the invention for the manufacture of a medicament for improving insulin release in a subject, for improving carbohydrate tolerance in a subject and/or improving carbohydrate metabolism in a subject. Such use may relate to treating subjects with a pre-diabetic state such as insulin insensitivity or pre-diabetes.

The invention also provides use of a compound, derivative or salt of the invention for the manufacture of a medicament for the reduction of appetite in a subject, reducing food intake in a subject, reducing calorie intake in a subject, improving insulin release in a subject, and/or use in improving carbohydrate tolerance in a subject.

The invention also provides use of a compound, derivative or salt of the invention for the manufacture of a medicament for providing cytoprotection (e.g. preventing or treating neurodegeneration, providing neuroprotection and/or providing cardiac protection) of a subject, who may be as described above in reference to other aspects of the invention.

According to certain embodiments the compound, derivative, salt or composition of the invention is to be administered parentally. According to other embodiments the compound, derivative, salt or composition of the invention is administered subcutaneously, intravenously, intramuscularly, intranasally, transdermally or sublingually. According to other embodiments the compound, derivative, salt or composition of the invention is administered orally. In one preferred embodiment compound, derivative, salt or composition of the invention is administered subcutaneously.

The compound, derivative, salt or composition of the invention is preferably used in the treatment of a human subject. However, while the compound, derivative, salt or composition of the invention will typically be used to treat human subjects they may also be used to treat similar or identical conditions in other vertebrates for example other primates; farm animals for example swine, cattle and poultry; sport animals for example horses; or companion animals for example dogs and cats.

### Compositions

It is preferable for the compound of formula (I), or the derivative and/or the salt thereof, to be present in a pharmaceutical formulation or composition. Accordingly, the invention provides a composition comprising a compound, derivative or salt of the invention together with a pharmaceutically acceptable excipient and optionally another therapeutic ingredient. Compositions comprising the compound, derivative or salt are suitable for pharmaceutical use. According to certain preferred embodiments the composition is present in a syringe or other administration device for subcutaneous administration to humans. According to certain preferred embodiments the composition has a pH of less than 5. Compositions of the invention may take the form of a pharmaceutical formulation as described below.

The pharmaceutical formulations according to the invention include those suitable for oral, parenteral (including subcutaneous, intradermal, intramuscular, intravenous, and intra-articular), inhalation (including fine particle dusts or mists which may be generated by means of various types of metered dose pressurized aerosols, nebulizers or insufflators), rectal and topical (including dermal, transdermal, transmucosal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988, the contents of which are incorporated herein by reference.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical compositions comprising the present compounds or, particularly in the case of extended release, by the use of devices such as subcutaneous implants or osmotic pumps. The present compounds may also be administered liposomally.

Preferably, compositions according to the invention are suitable for subcutaneous administration, for example by injection. According to certain embodiments the composition may contain metal ions, for example copper, iron, aluminium, zinc, nickel or cobalt ions. The presence of such ions may limit solubility and thus delay absorption into the circulatory system from the site of subcutaneous administration.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. Such compositions may also include a permeation enhancer. The compounds of the invention may also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxypropyl cellulose (HPC), hydroxypropyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor. An aqueous carrier may be, for example, an isotonic buffer solution at a pH of from about 3.0 to about 8.0, preferably at a pH of from about 3.5 to about 7.4, for example from 3.5 to 6.0, for example from 3.5 to about 5.0. Useful buffers include sodium citrate-citric acid and sodium phosphate-phosphoric acid, and sodium acetate/acetic acid buffers. The composition preferably does not include any compounds known to be deleterious to peptide compounds.

Excipients that can be included are, for instance, other proteins, such as human serum albumin or plasma preparations. If desired, the pharmaceutical composition may also contain minor amounts of non-toxic auxiliary substances, such as wetting or emulsifying agents, preservatives, and pH buffering agents and the like, for example sodium acetate or sorbitan monolaurate.

Exemplary compositions for nasal aerosol or inhalation administration include solutions in saline, which can contain, for example, benzyl alcohol or other suitable preservatives, absorption promoters to enhance bioavailability, and/or other solubilizing or dispersing agents such as those known in the art. Conveniently in compositions for nasal aerosol or inhalation administration the compound of the invention is delivered in the form of an aerosol spray presentation from a pressurized pack or a nebulizer, with the use of a suitable propellant, e.g., dichlorodifluoro-methane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated to contain a powder mix of the compound and a suitable powder base, for example lactose or starch. In one specific, non-limiting example, a compound of the invention is administered as an aerosol from a metered dose valve, through an aerosol adapter also known as an actuator. Optionally, a stabilizer is also included, and/or porous particles for deep lung delivery are included (e.g., see U.S. Pat. No. 6,447,743).

Formulations for rectal administration may be presented as a retention enema or a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

The compounds, derivatives and salts of the invention may also be suitably administered as sustained-release systems. Suitable examples of sustained-release systems of the invention include suitable polymeric materials, for example semi-permeable polymer matrices in the form of shaped articles, e.g., films, or microcapsules; suitable hydrophobic materials, for example as an emulsion in an acceptable oil; or ion exchange resins; and sparingly soluble derivatives of the compound of the invention, for example, a sparingly soluble salt. Sustained-release systems may be administered orally; rectally; parenterally; intracisternally; intravaginally; intraperitoneally; topically, for example as a powder, ointment, gel, drop or transdermal patch; bucally; or as an oral or nasal spray.

Preparations for administration can be suitably formulated to give controlled release of compounds, derivatives and salts of the invention. For example, the pharmaceutical compositions may be in the form of particles comprising one or more of biodegradable polymers, polysaccharide jellifying and/or bioadhesive polymers, amphiphilic polymers, agents capable of modifying the interface properties of particles of the compounds of the invention. These compositions exhibit certain biocompatibility features which allow a controlled release of the active substance, see U.S. Pat. No. 5,700,486, the contents of which are incorporated by reference.

The use of a controlled release composition is preferred for indications such as the treatment of obesity and/or diabetes, where maximising the time period between injections is desirable. However, for indications such as providing neuroprotection or cardiac protection (e.g. following suspected myocardial infarction or stroke), where it is desired to achieve a therapeutic plasma concentration of the active agent in as short a time period as possible, an immediate release formulation will be preferred. In such cases, a dosage regime comprising administration of a dose of an immediate release formulation of the active agent (i.e. as soon as possible after suspected myocardial infarction or stroke) and subsequent administration of a dose of a controlled release formulation of the active agent may be preferred.

A compound, derivative or salt of the invention may be delivered by way of a pump (see Langer, supra; Sefton, CRC Crit. Ref Biomed. Eng. 14:201, 1987; Buchwald et al., Surgery 88:507, 1980; Saudek et al., N. Engl. J. Med. 321:574, 1989) or by a continuous subcutaneous infusion, for example, using a mini-pump. An intravenous bag solution may also be employed. The key factor in selecting an appropriate dose is the result obtained, as measured by decreases in total body weight or ratio of fat to lean mass, or by other criteria for measuring control or prevention of obesity or prevention of obesity-related conditions, as are deemed appropriate by the practitioner. Other controlled release systems are discussed in the review by Langer (Science 249:1527-1533, 1990) which is incorporated herein by reference. In another aspect of the disclosure compounds of the invention are delivered by way of an implanted pump, described, for example, in U.S. Pat. Nos. 6,436,091; 5,939,380; 5,993,414, the contents of which are incorporated herein by reference.

Implantable drug infusion devices are used to provide patients with a constant and long-term dosage or infusion of a drug or any other therapeutic agent. Essentially such device may be categorized as either active or passive. A compound, derivative or salt of the present invention may be formulated as a depot preparation. Such a long acting depot formulation can be administered by implantation, for example subcutaneously or intramuscularly; or by intramuscular injection. Thus, for example, the active ingredient can be formulated with suitable polymeric or hydrophobic materials, for example as an emulsion in an acceptable oil; or ion exchange resins; or as a sparingly soluble derivatives, for example, as a sparingly soluble salt.

A therapeutically effective amount of the active agent of the invention may be administered as a single pulse dose, as a bolus dose, or as pulse doses administered over time. Thus, in pulse doses, a bolus administration of the active agent is provided, followed by a time period wherein no active agent is administered to the subject, followed by a second bolus administration. In specific, non-limiting examples, pulse doses are administered during the course of a day, during the course of a week, or during the course of a month.

### Combination Treatments

In certain embodiments, a therapeutically effective amount of a compound, derivative, salt or composition of the invention is administered with a therapeutically effective amount of a further agent or agents. The compound, derivative or salt may for example be administered simultaneously with one or more further therapeutic agent(s), or it may be administered sequentially or separately. Accordingly, the invention provides a compound, derivative or salt of the invention for use as a medicament, wherein the compound, derivative or salt is for use with a therapeutically effective amount of a further therapeutic agent or agents (e.g. for administration simultaneously, sequentially or separately). In certain embodiments, the active agent of the invention is formulated and administered with a further therapeutic agent or agents as a single dose.

In certain embodiments, the further therapeutic agent or agents is/are an additional anti-diabetic, appetite suppressant, a food-intake-reducing, plasma glucose-lowering or plasma lipid-altering agent. Specific, non-limiting examples of an additional appetite suppressant include amfepramone (diethylpropion), phentermine, mazindol and phenylpropanolamine, fenfluramine, dexfenfluramine, phendimetrazine, benzphetamine, sibutramine, rimonabant, topiramate, fluoxetine, bupropion, zonisamide, naltrexone, orlistat and cetilistat. Specific, non-limiting examples of an additional anti-diabetic agent include metformin, phenformin, rosiglitazone, pioglitazone, troglitazone, repaglinide, nateglinide, tolbutamide, acetohexamide, tolazamide, chlorpropamide, glipizide, glyburide, glimepiride, gliclazide, fibroblast growth factor 21, miglitol, acarbose, exenatide, pramlintide, vildagliptin and sitagliptin.

In alternative embodiments, the further therapeutic agent or agents is/are an additional cardioprotective or neuroprotective agent. Specific, non-limiting, examples of additional cardioprotective agents include aspirin, N-acetylcysteine, phenethylamines, coenzyme Q10, vitamin E, vitamin C, L-carnitine, carvedilol and dexrazoxane. Specific, non-limiting examples of neuroprotective agents include statins such as simvastatin, steroids such as progesterone, minocycline, resveratrol and vitamin E. Examples of agents used for the treatment of Parkinson's disease include anticholinergics, pramipexole, bromocriptine, levodopa, carbidopa, rasagiline, amantadine and ropinirole.

### Dosages

A compound, derivative, salt or composition of the invention may be administered whenever the effect, e.g., appetite suppression, decreased food intake or decreased caloric intake, is desired, or slightly before to whenever the effect is desired, such as, but not limited to, about 10 minutes, about 15 minutes, about 30 minutes, about 60 minutes, about 90 minutes, or about 120 minutes, before the time the effect is desired.

The therapeutically effective amount of the active agent of the invention will be dependent on the molecule utilized, the subject being treated, the severity and type of the affliction, and the manner and route of administration. For example, a therapeutically effective amount of a compound of the invention may vary from about 0.01 g per kilogram (kg) body weight to about 1 g per kg body weight, for example about 0.1 g to about 20 mg per kg body weight, for example about 1 g to about 5 mg per kg body weight, or about 5 g to about 1 mg per kg body weight.

In one embodiment of the invention, a compound, derivative or salt of the invention may be administered to a subject at from 0.5 to 1,333 nmol per kg bodyweight, for example 1 to 1,333 nmol/kg bodyweight, for example 2 to 1,000 nmol per kg bodyweight, for example 4 to 1,333 nmol per kg bodyweight, for example from 5 to 1,000 nmol per kg bodyweight, for example at from 10 to 750 nmol per kg bodyweight, for example at from 20 to 500 nmol per kg bodyweight, in particular at from 30 to 240 nmol per kg bodyweight. In a preferred embodiment, a high activity compound of the invention is administered to a subject at from 0.2 to 10 nmol per kg bodyweight, for example 0.5 to 5.0 nmol/kg bodyweight, for example 1.0 to 2.0 nmol per kg bodyweight, for example 1.5 nmol per kg bodyweight, For a 75 kg subject, such doses correspond to dosages of from 37.5 nmol to 100 μmol, for example from 75 nmol to 100 μmol, for example from 150 nmol to 100 μmol, for example from 300 nmol to 100 μmol, for example from 375 nmol to 75 μmol, for example from 750 nmol to 56.25 μmol, for example from 1.5 to 37.5 μmol, in particular from 2.25 to 18 μmol. In a preferred embodiment for a high activity compound of the invention, for a 75 kg subject, such doses correspond to dosages of from 15 to 750 nmol, for example 37.5 to 375.0 nmol, for example 75 to 150 nmol, for example 112.5 nmol. The invention also contemplates dosages ranges bounded by any of the specific dosages mentioned herein.

The exact dose is readily determined by one of skill in the art based on the potency of the specific compound utilized, the route of delivery of the compound and the age, weight, sex and physiological condition of the subject.

For a compound with a long blood half-life, the doses discussed above may be given, for example, once or twice per month, or once, twice, three-times or four-times per week. For a preferred compound, a dose may be given no more frequently than once a week. Alternatively, for a compound with a shorter half-life in the blood, the doses discussed above may be given, for example, once, twice, three-times or four-times a day or once or twice a week. In some embodiments, a dose may be given once every 2, 3 or 4 days. According to certain embodiments they may be administered once shortly before each meal to be taken.

### EXAMPLES

The invention is further described with reference to the following non-limiting examples.

### Materials and Methods

### Peptide Synthesis

Peptide synthesis was carried out on a tricyclic amide linker resin. Amino acids were attached using the Fmoc strategy. For the portion of the molecule from Xaa1 to Xaa42, each amino acid was added sequentially from the C- to the N-termini. Peptide couplings were mediated using reagents such as HBTU. Peptide cleavage from the resin was achieved with trifluoracetic acid in the presence of scavengers. In a second stage, the lysine 42 residue was functionalised at its ε-amino group following deprotection of the F-amino group. The chain on the lysine 42 residue was then constructed sequentially using the same amino acid attachment chemistry.

Peptides were purified by reverse phase HPLC. Quality control was performed on all purified peptides and peptides were shown in most cases to be greater than 90% pure by HPLC in two buffer systems. MALDI-MS showed the expected molecular ion.

### Example Synthesis

Example compound 193 (G Ref 6699) was prepared as follows using standard Fmoc chemistry:

Resin preparation: To 2Cl-Trt Resin (0.25 mmol, 1.00 eq) was added FMOC-LYS(DDE)-OH (133.15 mg, 250.00 μmol, 1.00 eq) and DIEA (193.85 mg, 1.50 mmol, 261.97 μL, 6.00 eq) in DCM (8.0 mL). The mixture was agitated with N₂ for 2 h at 20° C., then added MeOH (0.25 mL) and agitated with N₂ for another 30 min. The resin was washed with DMF (12.0 mL*3). Then 20% piperidine in DMF (5.00 mL) was added and the mixture was agitated with N₂ for 30 min at 20° C. Then the mixture was filtered to get the resin. The resin was washed with DMF (12.0 mL*5) and filtered to get the resin.

FMOC-LYS(DDE)-OH has the structure:

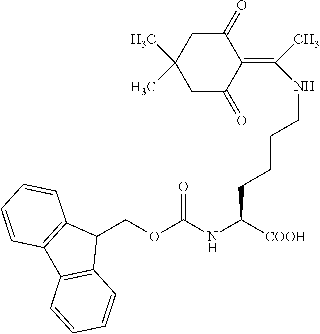 [SMILES: CC(NCCCC[C@H](NC(=O)OCC1c2ccccc2-c2ccccc21)C(=O)O)=C1C(=O)CC(C)(C)CC1=O]

The free acid is attached to the resin and the piperidine deprotection creates a free amino end group for the next coupling.
1. Coupling: a solution of FMOC-LYS(BOC)-OH (351.37 mg, 750.00 μmol, 3.00 eq), DIEA (193.85 mg, 1.50 mmol, 261.97 μL, 6.00 eq) and HBTU (270.20 mg, 712.50 μmol, 2.85 eq) in DMF (3.00 mL) was added to the resin and agitated with N₂ for 30 min at 20° C. The resin was then washed with DMF (12.0 mL*3).
2. Deprotection: 20% piperidine in DMF (5.00 mL) was added to the resin and the mixture was agitated with N₂ for 30 min at 20° C. The resin was washed with DMF (12.0 mL*5) and filtered to get the resin.
3. Steps 2-3 were repeated using the reagents in Table 1 until the last amino acid had been added (reaction iteration #1 in Table 1 is the first added Lys residue, as set out in steps 2 and 3 above).

TABLE 1

| # | Materials | Coupling reagents |
|---|---|---|
| 1 | FMOC-PRO-OH (3.00 eq) | HBTU (2.85 eq) and DIPEA (6.00 eq) |
| 2 | FMOC-PRO-OH (3.00 eq) | HBTU (2.85 eq) and DIPEA (6.00 eq) |
| 3 | FMOC-PRO-OH (3.00 eq) | HBTU (2.85 eq) and DIPEA (6.00 eq) |
| 4 | FMOC-LYS(BOC)-OH (3.00 eq) | HBTU (2.85 eq) and DIPEA (6.00 eq) |
| 5 | FMOC-GLY-OH (3.00 eq) | HBTU (2.85 eq) and DIPEA (6.00 eq) |
| 6 | FMOC-SER(TBU)-OH (3.00 eq) | HBTU (2.85 eq) and DIPEA (6.00 eq) |
| 7 | FMOC-SER(TBU)-OH (3.00 eq) | HBTU (2.85 eq) and DIPEA (6.00 eq) |
| 8 | FMOC-PRO-OH (3.00 eq) | HBTU (2.85 eq) and DIPEA (6.00 eq) |
| 9 | FMOC-GLY-OH (3.00 eq) | HBTU (2.85 eq) and DIPEA (6.00 eq) |
| 10 | FMOC-GLY-OH (3.00 eq) | HBTU (2.85 eq) and DIPEA (6.00 eq) |
| 11 | FMOC-ALA-OH (3.00 eq) | HBTU (2.85 eq) and DIPEA (6.00 eq) |
| 12 | FMOC-LYS(BOC)-OH (3.00 eq) | HBTU (2.85 eq) and DIPEA (6.00 eq) |
| 13 | FMOC-LEU-OH (3.00 eq) | HBTU (2.85 eq) and DIPEA (6.00 eq) |
| 14 | FMOC-TRP(BOC)-OH (3.00 eq) | HBTU (2.85 eq) and DIPEA (6.00 eq) |
| 15 | FMOC-GLU(OTBU)-OH (3.00 eq) | HBTU (2.85 eq) and DIPEA (6.00 eq) |
| 16 | FMOC-ILE-OH (3.00 eq) | HBTU (2.85 eq) and DIPEA (6.00 eq) |
| 17 | FMOC-PHE-OH (3.00 eq) | HBTU (2.85 eq) and DIPEA (6.00 eq) |
| 18 | FMOC-LEU-OH (3.00 eq) | HBTU (2.85 eq) and DIPEA (6.00 eq) |
| 19 | FMOC-ARG(PBF)-OH (3.00 eq) | HBTU (2.85 eq) and DIPEA (6.00 eq) |
| 20 | FMOC-VAL-OH (3.00 eq) | HBTU (2.85 eq) and DIPEA (6.00 eq) |
| 21 | FMOC-ARG(PBF)-OH (3.00 eq) | HBTU (2.85 eq) and DIPEA (6.00 eq) |
| 22 | FMOC-GLU(OTBU)-OH (3.00 eq) | HBTU (2.85 eq) and DIPEA (6.00 eq) |
| 23 | FMOC-GLU(OTBU)-OH (3.00 eq) | HBTU (2.85 eq) and DIPEA (6.00 eq) |
| 24 | FMOC-GLU(OTBU)-OH (3.00 eq) | HBTU (2.85 eq) and DIPEA (6.00 eq) |
| 25 | FMOC-LEU-OH (3.00 eq) | HBTU (2.85 eq) and DIPEA (6.00 eq) |
| 26 | FMOC-GLN(TRT)-OH (3.00 eq) | HBTU (2.85 eq) and DIPEA (6.00 eq) |
| 27 | FMOC-LYS(BOC)-OH (3.00 eq) | HBTU (2.85 eq) and DIPEA (6.00 eq) |
| 28 | FMOC-SER(TBU)-OH (3.00 eq) | HBTU (2.85 eq) and DIPEA (6.00 eq) |
| 29 | FMOC-TYR(TBU)-OH (3.00 eq) | HBTU (2.85 eq) and DIPEA (6.00 eq) |
| 30 | FMOC-ASP(OTBU)-OH (3.00 eq) | HBTU (2.85 eq) and DIPEA (6.00 eq) |
| 31 | FMOC-SER(TBU)-OH (3.00 eq) | HBTU (2.85 eq) and DIPEA (6.00 eq) |
| 32 | FMOC-THR(TBU)-OH (3.00 eq) | HBTU (2.85 eq) and DIPEA (6.00 eq) |
| 33 | FMOC-PHE-OH (3.00 eq) | HBTU (2.85 eq) and DIPEA (6.00 eq) |
| 34 | FMOC-THR(TBU)-OH (3.00 eq) | HBTU (2.85 eq) and DIPEA (6.00 eq) |
| 35 | FMOC-GLY-OH (3.00 eq) | HBTU (2.85 eq) and DIPEA (6.00 eq) |
| 36 | FMOC-HIS(TRT)-OH (3.00 eq) | HBTU (2.85 eq) and DIPEA (6.00 eq) |
| 37 | FMOC-AIB-OH (3.00 eq) | HBTU (2.85 eq) and DIPEA (6.00 eq) |
| 38 | BOC-HIS(TRT)-OH (3.00 eq) | HBTU (2.85 eq) and DIPEA (6.00 eq) |

5. After the coupling of BOC-HIS(TRT)-OH in iteration #38, 3% H₂N·NH₂/DMF was added and reacted for 30 min to remove Dde, and then repeated. The mixture was then drained and washed with DMF (15.0 mL) 5 times. After removal of the Dde group, the compound has a free amino end group at the F-amino group of the lysine from step 1 and that is available for the next coupling.
6. The reactions of steps 2-3 were then carried out using the reagents in Table 2 until the last reagent has been added (reaction iteration #40 in Table).

TABLE 2

| # | Materials | Coupling reagents |
|---|---|---|
| 39 | FMOC-GLU-OTBU (3.00 eq)* | HBTU (2.85 eq) and DIEA (6.00 eq) |
| 40 | 20-(tert-butoxy)-20- | HBTU (2.85 eq) and DIEA (6.00 eq) |
| | oxooctadecanoic acid (3.00 eq) | |

*As the protected glutamic acid reagent 39 has its C-1 acid group protected with TBU, it reacts at its C-5 acid with the ε-amino group of lysine 40. It is the FMOC-GLU-OTBU reagent that provides the glutamic acid residue portion in the Z part of the compound:

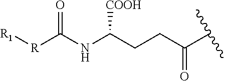

7. Peptide Cleavage and Purification:
   The resin was washed with MeOH (20.0 mL*2) and dried under vacuum to provide 2.0 g peptide resin. Then 22.0 mL of cleavage buffer (92.5% TFA/2.5% Mpr/2.5% TIS/2.5% H₂O) was added to the flask containing the side chain protected peptide resin at 20° C. and the mixture was stirred for 2.5 h. The peptide was precipitated with cold tert-butyl methylether (250 mL) and centrifuged (2 min at 3000 rpm). The peptide precipitate was washed with tert-butyl methyl ether twice more (100 mL). The crude peptide was dried and the identity confirmed by LCMS.
   The residue was purified by prep-HPLC (TFA condition; 30° C., A: 0.075% TFA/H₂O, B: CH₃CN) to give the title compound (202.4 mg, 40.66 μmol, 15.32% yield, 91.03% purity, TFA) as a white solid, the identity of which was confirmed by LCMS).

Equivalent methods were employed for all of the other peptides described herein. The sequences and other structural features of the exemplified peptides are shown in FIG. 1. In the FIGURE, the Lysine residue at position 42 is substituted on its F amino group with a group Y and Y is Z-Xaa43-Xaa44- (blank in the column in question means that the residue is absent), and Z is a group a having the structure:

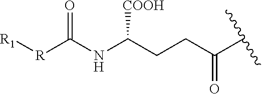

wherein R is a C₁₆-C₁₈ straight chain alkylene or alkenylene group; and
   R¹ is CO₂H.

The number of carbon atoms in the alkylene or alkenylene group of R (n, n=16-18) is indicated in the column headed “n (R=Cn)” in FIG. 1. In the examples shown, n is 16 or 18.

Certain of the example compounds in FIG. 1 are duplicate preparations of compounds listed elsewhere in the FIGURE and they are indicated as such as “dupl” in the column headed ‘Notes’.

### Receptor Potency of Peptides at the Human GLP-1 Receptor, Overexpressed in CHO Cells

Biological activity was assessed by potency of peptides to stimulate cAMP production in Chinese Hamster Ovary (CHO) cell lines overexpressing the human GLP receptor. Cells were plated at a density of 8×10⁻⁵ cells/mL in serum-free media, into 96 well half area plates, on the day of the assay. A commercial cAMP kit (Cisbio) was used to quantify cAMP in the cell via HTRF (Homogeneous Time-Resolved Fluorescence) technology after 30 mins of peptide stimulation and a further 1 h lysis. Plates were read on a SpectraMax i3x Multi-Mode Detection Platform plate reader and concentration response curves drawn with Graph Pad Prism 7.0 (or higher). EC₅₀ values were generated for each peptide and compared to the controls for the day.

### In Vivo Efficacy Study: Single Dose Feeding Studies in Male Rats

Male rats (Charles River Ltd, Margate, UK) were used for animal experiments. Ad libitum fed rats were individually housed in IVC cages. Animals were randomised into treatment groups, with stratification by body weight. All peptide solutions were prepared freshly immediately prior to administration. The control animals were dosed water 5% v/v water and 95% NaCl (0.9% w/v) whilst peptides (either 1.5, 2, 3 or 6 nmol/kg body weight) were resuspended in water for injection. Peptide and vehicle were administered in the early light phase (0900-1000) by subcutaneous injection and animals provided a known amount of food. Animals were given free access to food and water during the study period. Animal body weight and remaining food were weighed throughout the study, typically 24, 48, 72 and 96, and in some examples 168 h post dosing. The results presented are the figures for 4 (96 h) or 7 days (168 h) post dosing.

The results for Example compounds 1 to 527 are shown in FIG. 1. The rats were dosed with a single subcutaneous injection of peptide. The dose was generally 1.5-6 nmol/kg body weight. In the Table in FIG. 1, column ‘n’ shows the number of times the compound in question was tested. Each test generally involved the compound being given to a group of 5 animals. The compounds were assessed for their propensity to inhibit food intake and their ability to bring about body weight change. The summation of their actions is reported as a single value, called “potency” in FIG. 1. The compounds were compared with semaglutide/Ozempic. For “potency”, a score of 8 means that the compound causes the same effect as Ozempic. A score above 8 indicates that there is greater inhibition of food intake and reduction in body weight and/or reduction in body weight gain than achieved by the same dose of Ozempic, or a similar effect to Ozempic but achieved with a lower dose of the test compounds. These high activity levels allow a compound to be administered at a lower dose than for semaglutide/Ozempic. In some cases, the dose can be ⅙^{th}, ⅛^{th}, 1/10^{th} or even 1/12^{th} of the level of semaglutide/Ozempic.

### PK Measurements for Half-Life Assessment

Male large white pigs receive a single subcutaneous injection of the test compound at the start of study (0 hours). Repeated blood samples were taken from the pig over the proceeding 168 hours and plasma separated and stored at −20 to −80° C. Plasma concentration of the test compound was quantified using mass spectrum analysis. Time taken from tₘₐₓ to half of Cₘₐₓ was calculated and represents the half-life (t_{1/2}) of the compound. Typically, the presented t_{1/2} is the mean of four separate animals.

### Results

FIG. 1 is a table providing amino acid sequences and other structural information for the example compounds of the invention. For example, the amino acid sequence of example compound no. 1 is as follows [SEQ ID NO: 1]:

| | | | | | | | | | | | |
|---|---|---|---|---|---|---|---|---|---|---|---|
| His | AIB | His | Gly | Thr | Phe | Thr | Ser | Asp | Leu | Ser | Lys |
| Gln | Leu | Glu | Glu | Glu | Ala | Val | Arg | Leu | Phe | Ile | Glu |
| Trp | Leu | Lys | Asn | Gly | Gly | Pro | Ser | Ser | Gly | Ala | Pro |
| Pro | Pro | Lys | | | | | | | | | |

And the Lys residue at position 42 carries on its F-amino group a group Z, where
Z is a group of formula:

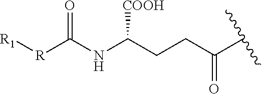

wherein R is a C₁₈ straight chain alkylene group; and
R¹ is CO₂H.

The FIGURE also summarises the results of the in vivo feeding efficacy studies with the example peptides of the invention as discussed above.

To the right of the columns showing the feeding scores, the FIGURE shows receptor potency data for example peptides at the GLP-1 receptor, overexpressed in Chinese hamster ovary (CHO) cells. Biological activity was assessed by the potency of peptides to stimulate cAMP production in the CHO cells as described above.

As can be seen from the FIGURE, rats which were given free access to food and which were administered example peptides of the invention, achieved reduced weight gain or achieved weight loss compared with rats which were administered saline. This supports that the compounds of the invention are particularly effective at improving metabolism, and that they find use in the therapy of disorders such as obesity. However, the amount of food consumed by rats which were administered the example peptides was similar to or greater than the amount of food consumed by rats which were administered saline. The absence of, or only minimal, effect on amount of food ingested supports that the compounds have reduced side effects relating to nausea. As discussed above, rodents are not able to vomit, but those experiencing nausea are likely to be put off from consuming food. With the peptides of the invention, there was no observed evidence of the animals being put off consuming food.

### Half-Life:

The half-life of a selection of compounds of the invention was measured. The results, in hours, are shown in the column labelled t %/2 at the far right of the table in FIG. 1.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

1. A compound having an amino acid sequence of Formula (I):
(I)
[SEQ ID NO: 528]
Xaa1-AIB2-Xaa3-Gly4-Thr5-Phe6-Thr7-Ser8-Asp9-
Xaa10-Ser11-Lys12-Gln13-Leu14-Glu15-Glu16-
Xaa17-Xaa18-Val19-Xaa20-Xaa21-Phe22-Ile23-Glu24-
Trp25-Leu26-Lys27-Xaa28-Xaa29-Gly30-Pro31-Ser32-
Xaa33-Gly34-Xaa35-Xaa36-Pro37-Pro38-Xaa39-Xaa40-
Xaa41-Lys42,
wherein
Xaa1 is His, Tyr or Phe;
Xaa3 is His, Gln or Glu;
Xaa10 is Leu, Tyr or Val;
Xaa17 is Glu or Lys;
Xaa18 is Ala or Arg;
Xaa20 is Arg, Gln, Lys or His;
Xaa21 is Leu, Ala, Arg, Lys or Glu;
Xaa28 is Ala, Asn, Gln or His;
Xaa29 is Gly or Ala;
Xaa33 is Ser, His or Gln;
Xaa35 is Lys, Pro, His, Arg, Asn, Gln, Glu, Ser or Ala;
Xaa36 is Phe, His, Gln, Glu, Lys, Ser, Arg or absent;
Xaa39 is Pro or Gly;
Xaa40 is Gly or absent; and
Xaa41 is Lys or absent.

2. The compound of claim 1, wherein
Xaa1 is His, Tyr or Phe;
Xaa3 is His, Gln or Glu;
Xaa10 is Leu, Tyr or Val;
Xaa17 is Lys;
Xaa18 is Ala or Arg;
Xaa20 is Arg or His;
Xaa21 is Leu or Glu;
Xaa28 is Ala, Gln or His;
Xaa29 is Gly;
Xaa33 is Ser;
Xaa35 is Lys, Glu, His, Gln or Asn;
Xaa36 is absent;
Xaa39 is Pro;
Xaa40 is Gly or absent; and
Xaa41 is Lys.

3. The compound of claim 1, wherein
Xaa1 is His or Phe;
Xaa3 is His or Glu;
Xaa10 is Tyr or Val;
Xaa17 is Lys;
Xaa18 is Ala or Arg;
Xaa20 is Arg or His;
Xaa21 is Glu;
Xaa28 is Ala or Gln;
Xaa29 is Gly;
Xaa33 is Ser;
Xaa35 is Lys;
Xaa36 is absent;
Xaa39 is Pro;
Xaa40 is Gly; and
Xaa41 is Lys.

4. The compound of claim 1 having an amino acid sequence comprising any one of SEQ ID NOs: 1 to 527.

5. The compound of claim 1 having an amino acid sequence comprising SEQ ID NO: 502.


SEQUENCE LISTING (536 sequences)

SEQ ID NO: 1 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEEAVRLFIEWLKNGGPSSGAPPPK

SEQ ID NO: 2 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEERVRLFIEWLKNGGPSSGAPPPK

SEQ ID NO: 3 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEEAVREFIEWLKNGGPSSGAPPPK

SEQ ID NO: 4 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEEAVREFIEWLKHGGPSSGAPPPK

SEQ ID NO: 5 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEEAVREFIEWLKQGGPSSGAPPPK

SEQ ID NO: 6 - protein, 39 aa, synthetic construct; identical to SEQ ID NO: 2
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 7 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEERVREFIEWLKNGGPSSGAPPPK

SEQ ID NO: 8 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEEAVRLFIEWLKNGGPSSGAPPPGK

SEQ ID NO: 9 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEEAVREFIEWLKNGGPSSGAPPPGK

SEQ ID NO: 10 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVREFIEWLKNGGPSSGAPPPGK

SEQ ID NO: 11 - protein, 40 aa, synthetic construct; identical to SEQ ID NO: 10
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 12 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVREFIEWLKHGGPSSGAPPPGK

SEQ ID NO: 13 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVHEFIEWLKNGGPSSGAPPPGK

SEQ ID NO: 14 - protein, 39 aa, synthetic construct; identical to SEQ ID NO: 2
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 15 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKNGGPSSGAPPPGK

SEQ ID NO: 16 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKHGGPSSGAPPPGK

SEQ ID NO: 17 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKHGGPSHGAPPPGK

SEQ ID NO: 18 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKHGGPSSGHPPPGK

SEQ ID NO: 19 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKNGGPSHGHPPPGK

SEQ ID NO: 20 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKNGGPSHGAPPPGK

SEQ ID NO: 21 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKNGGPSSGHPPPGK

SEQ ID NO: 22 - protein, 39 aa, synthetic construct; identical to SEQ ID NO: 1
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 23 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
FXHGTFTSDLSKQLEEEAVRLFIEWLKNGGPSSGAPPPK

SEQ ID NO: 24 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEERVRLFIEWLKNGGPSSGHPPPK

SEQ ID NO: 25 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEERVRLFIEWLKNGGPSSGKPPPK

SEQ ID NO: 26 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEERVRLFIEWLKNGGPSHGAPPPK

SEQ ID NO: 27 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEERVRLFIEWLKNGGPSHGHPPPK

SEQ ID NO: 28 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEEAVREFIEWLKNGGPSHGHPPPK

SEQ ID NO: 29 - protein, 39 aa, synthetic construct; identical to SEQ ID NO: 27
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 30 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEERVREFIEWLKNGGPSHGHPPPK

SEQ ID NO: 31 - protein, 39 aa, synthetic construct; identical to SEQ ID NO: 27
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 32 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEEAVRLFIEWLKNGGPSHGHPPPK

SEQ ID NO: 33 - protein, 39 aa, synthetic construct; identical to SEQ ID NO: 32
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 34 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKNGGPSSGAPPPGK

SEQ ID NO: 35 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKAVRLFIEWLKNGGPSSGAPPPGK

SEQ ID NO: 36 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEKRVRLFIEWLKNGGPSSGAPPPK

SEQ ID NO: 37 - protein, 40 aa, synthetic construct; identical to SEQ ID NO: 34
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 38 - protein, 40 aa, synthetic construct; identical to SEQ ID NO: 34
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 39 - protein, 40 aa, synthetic construct; identical to SEQ ID NO: 8
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 40 - protein, 40 aa, synthetic construct; identical to SEQ ID NO: 15
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 41 - protein, 40 aa, synthetic construct; identical to SEQ ID NO: 21
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 42 - protein, 39 aa, synthetic construct; identical to SEQ ID NO: 36
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 43 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEKRVRLFIEWLKNGGPSHGHPPPK

SEQ ID NO: 44 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEKRVRLFIEWLKNGGPSSGHPPPK

SEQ ID NO: 45 - protein, 39 aa, synthetic construct; identical to SEQ ID NO: 24
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 46 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKNGGPSSGHPPPGK

SEQ ID NO: 47 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEEAVRLFIEWLKNGGPSSGHPPPGK

SEQ ID NO: 48 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKAVRLFIEWLKNGGPSSGHPPPGK

SEQ ID NO: 49 - protein, 40 aa, synthetic construct; identical to SEQ ID NO: 46
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 50 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEERVRLFIEWLKNGGPSSGAPPGK

SEQ ID NO: 51 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEKRVRLFIEWLKNGGPSSGHPPGK

SEQ ID NO: 52 - protein, 40 aa, synthetic construct; identical to SEQ ID NO: 34
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 53 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEKAVRLFIEWLKNGGPSSGAPPGK

SEQ ID NO: 54 - protein, 40 aa, synthetic construct; identical to SEQ ID NO: 34
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 55 - protein, 39 aa, synthetic construct; identical to SEQ ID NO: 36
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 56 - protein, 40 aa, synthetic construct; identical to SEQ ID NO: 34
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 57 - protein, 40 aa, synthetic construct; identical to SEQ ID NO: 46
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 58 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKNGGPSSGKPPPGK

SEQ ID NO: 59 - protein, 40 aa, synthetic construct; identical to SEQ ID NO: 58
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 60 - protein, 39 aa, synthetic construct; identical to SEQ ID NO: 36
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 61 - protein, 40 aa, synthetic construct; identical to SEQ ID NO: 34
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 62 - protein, 40 aa, synthetic construct; identical to SEQ ID NO: 34
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 63 - protein, 39 aa, synthetic construct; identical to SEQ ID NO: 36
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 64 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKQGGPSSGAPPPGK

SEQ ID NO: 65 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEKRVRLFIEWLKNGGPSSGPPPGK

SEQ ID NO: 66 - protein, 40 aa, synthetic construct; identical to SEQ ID NO: 34
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 67 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKNAGPSSGAPPPGK

SEQ ID NO: 68 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKQGGPSSGAPPPGK

SEQ ID NO: 69 - protein, 40 aa, synthetic construct; identical to SEQ ID NO: 68
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 70 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKNGGPSSGKPPPKK

SEQ ID NO: 71 - protein, 40 aa, synthetic construct; identical to SEQ ID NO: 70
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 72 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKNGGPSSGAPPPKK

SEQ ID NO: 73 - protein, 40 aa, synthetic construct; identical to SEQ ID NO: 72
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 74 - protein, 40 aa, synthetic construct
  REGION 1..40: note = Component of peptide hormone analogue
  MOD_RES 2: note = Aib
  MOD_RES 40: note = The residue at this position is Lys modified with a Y2 group attached to its epsilon amino group
HXHGTFTSDLSKQLEEKRVRLFIEWLKAGGPSSGAPPPGK

SEQ ID NO: 75 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEKRVRLFIEWLKQGGPSSGAPPPK

SEQ ID NO: 76 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKAGGPSSGAPPPGK

SEQ ID NO: 77 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDYSKQLEEKRVRLFIEWLKQGGPSSGAPPPK

SEQ ID NO: 78 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKNAGPSSGAPPPGK

SEQ ID NO: 79 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKQGGPSSGKPPPGK

SEQ ID NO: 80 - protein, 40 aa, synthetic construct; identical to SEQ ID NO: 79
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 81 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKAVRLFIEWLKQGGPSSGKPPPGK

SEQ ID NO: 82 - protein, 40 aa, synthetic construct; identical to SEQ ID NO: 81
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 83 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKQGGPSSGKPPPGK

SEQ ID NO: 84 - protein, 40 aa, synthetic construct; identical to SEQ ID NO: 83
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 85 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKAVRLFIEWLKQGGPSSGHPPPGK

SEQ ID NO: 86 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKQGGPSSGHPPPGK

SEQ ID NO: 87 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKQGGPSSGHPPPGK

SEQ ID NO: 88 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEKRVRLFIEWLKQGGPSSGHPPPK

SEQ ID NO: 89 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKNAGPSSGHPPPGK

SEQ ID NO: 90 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDLSKQLEEKRVRLFIEWLKNAGPSSGHPPPK

SEQ ID NO: 91 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKHGGPSSGAPPPGK

SEQ ID NO: 92 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKHGGPSSGAPPPGK

SEQ ID NO: 93 - protein, 40 aa, synthetic construct; identical to SEQ ID NO: 91
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 94 - protein, 40 aa, synthetic construct; identical to SEQ ID NO: 92
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 95 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKHGGPSSGKPPPGK

SEQ ID NO: 96 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKHGGPSSGHPPPGK

SEQ ID NO: 97 - protein, 39 aa, synthetic construct; identical to SEQ ID NO: 44
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 98 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVREFIEWLKQGGPSSGAPPPGK

SEQ ID NO: 99 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVHEFIEWLKQGGPSSGAPPPGK

SEQ ID NO: 100 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVREFIEWLKQGGPSSGHPPPGK

SEQ ID NO: 101 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVHEFIEWLKQGGPSSGHPPPGK

SEQ ID NO: 102 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKQGGPSHGHPPPGK

SEQ ID NO: 103 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVHLFIEWLKQGGPSSGHPPPGK

SEQ ID NO: 104 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKQGGPSSGHPPPGK

SEQ ID NO: 105 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVQEFIEWLKQGGPSSGHPPPGK

SEQ ID NO: 106 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKNAGPSSGEPPPGK

SEQ ID NO: 107 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKNAGPSSGHPPPGK

SEQ ID NO: 108 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVRLFIEWLKNAGPSSGAPPPKK

SEQ ID NO: 109 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVRLFIEWLKAGGPSSGAPPPKK

SEQ ID NO: 110 - protein, 40 aa, synthetic construct; identical to SEQ ID NO: 109
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 111 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVRLFIEWLKAGGPSSGAPPPGK

SEQ ID NO: 112 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKAGGPSSGKPPPGK

SEQ ID NO: 113 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKAGGPSSGAPPPKK

SEQ ID NO: 114 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVRLFIEWLKAGGPSSGAPPPKK

SEQ ID NO: 115 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKAGGPSSGKPPPKK

SEQ ID NO: 116 - protein, 40 aa, synthetic construct; identical to SEQ ID NO: 114
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 117 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVRLFIEWLKAGGPSSGHPPPKK

SEQ ID NO: 118 - protein, 40 aa, synthetic construct; identical to SEQ ID NO: 114
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 119 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVRLFIEWLKAGGPSSGKPPPKK

SEQ ID NO: 120 - protein, 40 aa, synthetic construct; identical to SEQ ID NO: 117
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 121 - protein, 40 aa, synthetic construct; identical to SEQ ID NO: 117
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 122 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKNAGPSSGHPPPKK

SEQ ID NO: 123 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKNAGPSSGHPPPKK

SEQ ID NO: 124 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKNAGPSSGKPPPKK

SEQ ID NO: 125 - protein, 40 aa, synthetic construct; identical to SEQ ID NO: 122
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 126 - protein, 40 aa, synthetic construct; identical to SEQ ID NO: 123
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 127 - protein, 40 aa, synthetic construct; identical to SEQ ID NO: 124
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 128 - protein, 40 aa, synthetic construct; identical to SEQ ID NO: 115
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 129 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVRLFIEWLKAGGPSSGKPPPGKK

SEQ ID NO: 130 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVRLFIEWLKAGGPSSGHPPPGKK

SEQ ID NO: 131 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKHGGPSSGKPPPKK

SEQ ID NO: 132 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVRLFIEWLKHGGPSSGKPPPGKK

SEQ ID NO: 133 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVRLFIEWLKHGGPSSGHPPPGKK

SEQ ID NO: 134 - protein, 40 aa, synthetic construct; identical to SEQ ID NO: 131
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 135 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKHGGPSSGHPPPKK

SEQ ID NO: 136 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKHGGPSSGAPPPKK

SEQ ID NO: 137 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKHGGPSSGKPPPKK

SEQ ID NO: 138 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKHGGPSSGHPPPKK

SEQ ID NO: 139 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVHEFIEWLKHGGPSSGKPPPKK

SEQ ID NO: 140 - protein, 40 aa, synthetic construct; identical to SEQ ID NO: 139
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 141 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKHGGPSSGKPPPKK

SEQ ID NO: 142 - protein, 40 aa, synthetic construct; identical to SEQ ID NO: 141
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 143 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKAGGPSSGKPPPKK

SEQ ID NO: 144 - protein, 40 aa, synthetic construct; identical to SEQ ID NO: 143
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 145 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKNAGPSSGKPPPKK

SEQ ID NO: 146 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKNAGPSSGKPPPGK

SEQ ID NO: 147 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDYSKQLEEKRVRLFIEWLKNAGPSSGKPPGK

SEQ ID NO: 148 - protein, 39 aa, synthetic construct; identical to SEQ ID NO: 147
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 149 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKNAGPSSGAPPPKK

SEQ ID NO: 150 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKNAGPSSGKPPPGK

SEQ ID NO: 151 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKNAGPSSGAPPPGK

SEQ ID NO: 152 - protein, 40 aa, synthetic construct; identical to SEQ ID NO: 151
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 153 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDYSKQLEEERVRLFIEWLKNAGPSSGKPPGK

SEQ ID NO: 154 - protein, 39 aa, synthetic construct; identical to SEQ ID NO: 153
  REGION 1..39: note = Component of peptide hormone analogue
  MOD_RES 2: note = Aib
  MOD_RES 39: note = The residue at this position is Lys modified with a Y group attached to its epsilon amino group

SEQ ID NO: 155 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKNAGPSSGKPPPKK

SEQ ID NO: 156 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKNAGPSSGRPPPKK

SEQ ID NO: 157 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKNAGPSSGRPPPGK

SEQ ID NO: 158 - protein, 40 aa, synthetic construct; identical to SEQ ID NO: 107
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 159 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKNAGPSSGHPPPGK

SEQ ID NO: 160 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKNAGPSSGKPPPGK

SEQ ID NO: 161 - protein, 40 aa, synthetic construct; identical to SEQ ID NO: 160
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 162 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKAGGPSSGAPPPGK

SEQ ID NO: 163 - protein, 40 aa, synthetic construct; identical to SEQ ID NO: 162
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 164 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKAGGPSSGHPPPGK

SEQ ID NO: 165 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKAGGPSSGAPPPGK

SEQ ID NO: 166 - protein, 40 aa, synthetic construct; identical to SEQ ID NO: 164
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 167 - protein, 40 aa, synthetic construct; identical to SEQ ID NO: 164
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 168 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKAGGPSSGHPPPKK

SEQ ID NO: 169 - protein, 40 aa, synthetic construct; identical to SEQ ID NO: 168
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 170 - protein, 40 aa, synthetic construct; identical to SEQ ID NO: 168
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 171 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVRLFIEWLKAGGPSSGHPPPGKK

SEQ ID NO: 172 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKAGGPSSGKPPPGK

SEQ ID NO: 173 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKAGGPSSGAPPPKK

SEQ ID NO: 174 - protein, 40 aa, synthetic construct; identical to SEQ ID NO: 168
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 175 - protein, 40 aa, synthetic construct; identical to SEQ ID NO: 173
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 176 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKHGGPSSGKPPPKK

SEQ ID NO: 177 - protein, 40 aa, synthetic construct; identical to SEQ ID NO: 123
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 178 - protein, 40 aa, synthetic construct; identical to SEQ ID NO: 176
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 179 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVRLFIEWLKHGGPSSGKPPPGKK

SEQ ID NO: 180 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVRLFIEWLKHGGPSSGAPPPGKK

SEQ ID NO: 181 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVRLFIEWLKHGGPSSGNPPPGKK

SEQ ID NO: 182 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVRLFIEWLKHGGPSSGEPPPGKK

SEQ ID NO: 183 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVRLFIEWLKHGGPSSGQPPPGKK

SEQ ID NO: 184 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVRLFIEWLKHGGPSSGSPPPGKK

SEQ ID NO: 185 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKHGGPSSGHPPPKK

SEQ ID NO: 186 - protein, 41 aa, synthetic construct; identical to SEQ ID NO: 179
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 187 - protein, 41 aa, synthetic construct; identical to SEQ ID NO: 133
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 188 - protein, 41 aa, synthetic construct; identical to SEQ ID NO: 179
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 189 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVRLFIEWLKHGGPSSGHPPPGKK

SEQ ID NO: 190 - protein, 41 aa, synthetic construct; identical to SEQ ID NO: 189
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 191 - protein, 40 aa, synthetic construct; identical to SEQ ID NO: 172
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 192 - protein, 40 aa, synthetic construct; identical to SEQ ID NO: 112
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 193 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKAGGPSSGKPPPKK

SEQ ID NO: 194 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKNAGPSSGHPPPKK

SEQ ID NO: 195 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVRLFIEWLKNAGPSSGHPPPGKK

SEQ ID NO: 196 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVRLFIEWLKAGGPSSGKPPPGKK

SEQ ID NO: 197 - protein, 41 aa, synthetic construct; identical to SEQ ID NO: 196
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 198 - protein, 41 aa, synthetic construct; identical to SEQ ID NO: 171
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 199 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKQGGPSSGHPPPGK

SEQ ID NO: 200 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHLFIEWLKHGGPSSGHPPPGK

SEQ ID NO: 201 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHLFIEWLKAGGPSSGHPPPGK

SEQ ID NO: 202 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHLFIEWLKHGGPSSGKPPPKK

SEQ ID NO: 203 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKHGGPSSGKPPPKK

SEQ ID NO: 204 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKHGGPSSGHPPPKK

SEQ ID NO: 205 - protein, 40 aa, synthetic construct; identical to SEQ ID NO: 204
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 206 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKNAGPSSGHPPPKK

SEQ ID NO: 207 - protein, 39 aa, synthetic construct
  REGION 1..39: note = Component of peptide hormone analogue
  MOD_RES 2: note = Aib
  MOD_RES 39: note = The residue at this position is Lys modified with a Y group attached to its epsilon amino group
HXHGTFTSDYSKQLEEERVHLFIEWLKAGGPSSGHPPPK

SEQ ID NO: 208 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVHLFIEWLKHGGPSSGHPPPGKK

SEQ ID NO: 209 - protein, 41 aa, synthetic construct; identical to SEQ ID NO: 208
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 210 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVHLFIEWLKAGGPSSGHPPPGKK

SEQ ID NO: 211 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVHLFIEWLKAGGPSSGKPPPGKK

SEQ ID NO: 212 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHEFIEWLKQGGPSSGHPPPGK

SEQ ID NO: 213 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHEFIEWLKAGGPSSGHPPPGK

SEQ ID NO: 214 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHEFIEWLKHGGPSSGHPPPGK

SEQ ID NO: 215 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVHEFIEWLKQGGPSSGHPPPGKK

SEQ ID NO: 216 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVREFIEWLKNAGPSSGAPPPGK

SEQ ID NO: 217 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKHGGPSSGHPPPKK

SEQ ID NO: 218 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKHGGPSSGHPPPGK

SEQ ID NO: 219 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHEFIEWLKHGGPSSGHPPPKK

SEQ ID NO: 220 - protein, 40 aa, synthetic construct; identical to SEQ ID NO: 214
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 221 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHEFIEWLKHGGPSSGKPPPKK

SEQ ID NO: 222 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKAGGPSSGAPPPGK

SEQ ID NO: 223 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKHGGPSSGAPPPGK

SEQ ID NO: 224 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKAGGPSSGKPPPKK

SEQ ID NO: 225 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHEFIEWLKAGGPSSGKPPPKK

SEQ ID NO: 226 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHEFIEWLKAGGPSSGKPPPKK

SEQ ID NO: 227 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHLFIEWLKAGGPSSGKPPPKK

SEQ ID NO: 228 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHEFIEWLKAGGPSSGHPPPKK

SEQ ID NO: 229 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVREFIEWLKAGGPSSGHPPPKK

SEQ ID NO: 230 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVREFIEWLKAGGPSSGKPPPKK

SEQ ID NO: 231 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVREFIEWLKAGGPSSGNPPPKK

SEQ ID NO: 232 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVREFIEWLKAGGPSSGEPPPKK

SEQ ID NO: 233 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVREFIEWLKAGGPSSGQPPPKK

SEQ ID NO: 234 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKAGGPSSGHPPPKK

SEQ ID NO: 235 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKAGGPSSGKPPPGK

SEQ ID NO: 236 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKAGGPSSGNPPPKK

SEQ ID NO: 237 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKAGGPSSGEPPPKK

SEQ ID NO: 238 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKAGGPSSGQPPPKK

SEQ ID NO: 239 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHLFIEWLKAGGPSSGEPPPGK

SEQ ID NO: 240 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHLFIEWLKAGGPSSGQPPPGK

SEQ ID NO: 241 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKNGGPSSGAPPPGK

SEQ ID NO: 242 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKNGGPSSGHPPPGK

SEQ ID NO: 243 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKQGGPSSGAPPPGK

SEQ ID NO: 244 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKQGGPSSGHPPPGK

SEQ ID NO: 245 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHEFIEWLKHGGPSSGHPPPGK

SEQ ID NO: 246 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHLFIEWLKQGGPSSGHPPPGK

SEQ ID NO: 247 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHEFIEWLKAGGPSSGHPPPGK

SEQ ID NO: 248 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKAGGPSSGHPPPGK

SEQ ID NO: 249 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKHGGPSSGHPPPGK

SEQ ID NO: 250 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVREFIEWLKAGGPSSGHPPPGKK

SEQ ID NO: 251 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVREFIEWLKAGGPSSGKPPPGKK

SEQ ID NO: 252 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVHEFIEWLKAGGPSSGHPPPGKK

SEQ ID NO: 253 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVHEFIEWLKAGGPSSGKPPPGKK

SEQ ID NO: 254 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVREFIEWLKHGGPSSGKPPPKK

SEQ ID NO: 255 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVREFIEWLKHGGPSSGKPPPKK

SEQ ID NO: 256 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVHEFIEWLKHGGPSSGKPPPKK

SEQ ID NO: 257 - protein, 40 aa, synthetic construct; identical to SEQ ID NO: 221
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 258 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVHEFIEWLKAGGPSSGKPPPGKK

SEQ ID NO: 259 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHLFIEWLKAGGPSSGKPPPGKK

SEQ ID NO: 260 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHLFIEWLKAGGPSSGKPPPKK

SEQ ID NO: 261 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHEFIEWLKAGGPSSGKPPPGKK

SEQ ID NO: 262 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEERVHLFIEWLKAGGPSSGKSPPPGKK

SEQ ID NO: 263 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEERVHLFIEWLKAGGPSSGQSPPPGKK

SEQ ID NO: 264 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEERVHLFIEWLKAGGPSSGHSPPPGKK

SEQ ID NO: 265 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVKLFIEWLKAGGPSSGKPPPGKK

SEQ ID NO: 266 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVKEFIEWLKAGGPSSGKPPPGKK

SEQ ID NO: 267 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVQLFIEWLKAGGPSSGKPPPGKK

SEQ ID NO: 268 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVRLFIEWLKAGGPSSGHPPPGK

SEQ ID NO: 269 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVRLFIEWLKAGGPSSGKPPPGK

SEQ ID NO: 270 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHLFIEWLKAGGPSSGKPPPGK

SEQ ID NO: 271 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHLFIEWLKAGGPSSGEPPPGKK

SEQ ID NO: 272 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKAGGPSSGHPPPGK

SEQ ID NO: 273 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHLFIEWLKAGGPSSGHPPPGK

SEQ ID NO: 274 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHLFIEWLKAGGPSSGNPPPGK

SEQ ID NO: 275 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVRLFIEWLKAGGPSSGEPPPGK

SEQ ID NO: 276 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVKLFIEWLKAGGPSSGEPPPGK

SEQ ID NO: 277 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVKLFIEWLKAGGPSSGEPPPGK

SEQ ID NO: 278 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKAGGPSHGHPPPGK

SEQ ID NO: 279 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVRLFIEWLKAGGPSHGHHPPPGK

SEQ ID NO: 280 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHLFIEWLKAGGPSHGHPPPGK

SEQ ID NO: 281 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKAGGPSSGEPPPGK

SEQ ID NO: 282 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKAGGPSSGEPPPGK

SEQ ID NO: 283 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKQGGPSSGEPPPGK

SEQ ID NO: 284 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHLFIEWLKQGGPSSGEPPPGK

SEQ ID NO: 285 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSSGEPPPGK

SEQ ID NO: 286 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHLFIEWLKAGGPSSGEPPPGK

SEQ ID NO: 287 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKHGGPSSGEPPPGK

SEQ ID NO: 288 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHLFIEWLKHGGPSSGEPPPGK

SEQ ID NO: 289 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSHGHPPPGK

SEQ ID NO: 290 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHLFIEWLKAGGPSHGHPPPGK

SEQ ID NO: 291 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHEFIEWLKAGGPSSGEPPPGK

SEQ ID NO: 292 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHEFIEWLKAGGPSHGHPPPGK

SEQ ID NO: 293 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHEFIEWLKHGGPSSGAPPPGK

SEQ ID NO: 294 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHLFIEWLKHGGPSSGHPPPGK

SEQ ID NO: 295 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHEFIEWLKAGGPSSGEPPPGKK

SEQ ID NO: 296 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHEFIEWLKAGGPSSGHPPPGKK

SEQ ID NO: 297 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHEFIEWLKHGGPSSGKPPPGKK

SEQ ID NO: 298 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVHLFIEWLKAGGPSSGKPPPGKK

SEQ ID NO: 299 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKAGGPSSGKPPPGKK

SEQ ID NO: 300 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHLFIEWLKAGGPSSGHPPPGKK

SEQ ID NO: 301 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKAGGPSSGKPPPGKK

SEQ ID NO: 302 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVHLFIEWLKAGGPSSGEPPPGKK

SEQ ID NO: 303 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKAGGPSSGEPPPGKK

SEQ ID NO: 304 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKAGGPSSGHPPPGKK

SEQ ID NO: 305 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSSGAPPPGK

SEQ ID NO: 306 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSSGHPPPGK

SEQ ID NO: 307 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSSGQPPPGK

SEQ ID NO: 308 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKQGGPSSGEPPPGKK

SEQ ID NO: 309 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKQGGPSSGHPPPGKK

SEQ ID NO: 310 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKHGGPSSGHPPPGKK

SEQ ID NO: 311 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKQGGPSSGKPPPGKK

SEQ ID NO: 312 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVRLFIEWLKAGGPSSGNPPPKK

SEQ ID NO: 313 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVHEFIEWLKAGGPSSGNPPPKK

SEQ ID NO: 314 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVHEFIEWLKHGGPSSGNPPPKK

SEQ ID NO: 315 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKHGGPSSGNPPPKK

SEQ ID NO: 316 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKQGGPSHGNPPPGK

SEQ ID NO: 317 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHEFIEWLKAGGPSSGNPPPGKK

SEQ ID NO: 318 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKQGGPSSGNPPPGK

SEQ ID NO: 319 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVREFIEWLKAGGPSSGNPPPGK

SEQ ID NO: 320 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVHEFIEWLKAGGPSSGNPPPGKK

SEQ ID NO: 321 - protein, 41 aa, synthetic construct; identical to SEQ ID NO: 181
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 322 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVKLFIEWLKAGGPSSGNPPPGK

SEQ ID NO: 323 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVRLFIEWLKAGGPSSGNPPPGK

SEQ ID NO: 324 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVKEFIEWLKAGGPSSGKPPPGKK

SEQ ID NO: 325 - protein, 41 aa, synthetic construct; identical to SEQ ID NO: 261
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 326 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHLFIEWLKAGGPSSGNPPPGKK

SEQ ID NO: 327 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKAGGPSSGNPPPKK

SEQ ID NO: 328 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKAGGPSSGEPPPKK

SEQ ID NO: 329 - protein, 41 aa, synthetic construct; identical to SEQ ID NO: 295
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 330 - protein, 40 aa, synthetic construct; identical to SEQ ID NO: 117
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 331 - protein, 40 aa, synthetic construct; identical to SEQ ID NO: 193
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 332 - protein, 40 aa, synthetic construct; identical to SEQ ID NO: 228
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 333 - protein, 40 aa, synthetic construct; identical to SEQ ID NO: 139
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 334 - protein, 40 aa, synthetic construct; identical to SEQ ID NO: 225
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 335 - protein, 40 aa, synthetic construct; identical to SEQ ID NO: 137
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 336 - protein, 41 aa, synthetic construct; identical to SEQ ID NO: 129
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 337 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHEFIEWLKAGGPSHGKPPPGKK

SEQ ID NO: 338 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHEFIEWLKAGGPSHGEPPPGKK

SEQ ID NO: 339 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHEFIEWLKAGGPSHGNPPPGKK

SEQ ID NO: 340 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHEFIEWLKAGGPSHGHPPPGKK

SEQ ID NO: 341 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVRLFIEWLKAGGPSSGHPPPGKK

SEQ ID NO: 342 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKAGGPSSGHPPPGKK

SEQ ID NO: 343 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKAVHEFIEWLKAGGPSHGKHPPPGKK

SEQ ID NO: 344 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKRVREFIEWLKAGGPSHGHHPPPGKK

SEQ ID NO: 345 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKAVHEFIEWLKAGGPSHGHKPPPGKK

SEQ ID NO: 346 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKAVHEFIEWLKAGGPSHGHEPPPGKK

SEQ ID NO: 347 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSHGNPPPGK

SEQ ID NO: 348 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKAGGPSHGNPPPGK

SEQ ID NO: 349 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKAVHEFIEWLKAGGPSHGNPPPGK

SEQ ID NO: 350 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKAVRLFIEWLKAGGPSSGHPPPKK

SEQ ID NO: 351 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEERVRLFIEWLKAGGPSSGKPPPKK

SEQ ID NO: 352 - protein, 41 aa, synthetic construct; identical to SEQ ID NO: 196
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 353 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVREFIEWLKAGGPSSGHPPPKK

SEQ ID NO: 354 - protein, 40 aa, synthetic construct; identical to SEQ ID NO: 230
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 355 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVKEFIEWLKAGGPSSGKPPPKK

SEQ ID NO: 356 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVKLFIEWLKAGGPSSGHPPPKK

SEQ ID NO: 357 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVKLFIEWLKAGGPSSGKPPPKK

SEQ ID NO: 358 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHLFIEWLKAGGPSSGHPPPKK

SEQ ID NO: 359 - protein, 40 aa, synthetic construct; identical to SEQ ID NO: 224
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 360 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKAGGPSSGHPPPKK

SEQ ID NO: 361 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDVSKQLEEKAVRLFIEWLKAGGPSSGHPPPKK

SEQ ID NO: 362 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDVSKQLEEERVRLFIEWLKAGGPSSGKPPPKK

SEQ ID NO: 363 - protein, 40 aa, synthetic construct; identical to SEQ ID NO: 119
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 364 - protein, 40 aa, synthetic construct; identical to SEQ ID NO: 117
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 365 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKAVRLFIEWLKAGGPSSGNPPPKK

SEQ ID NO: 366 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKAVRLFIEWLKAGGPSSGEPPPKK

SEQ ID NO: 367 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSSGKPPPKK

SEQ ID NO: 368 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSSGHPPPKK

SEQ ID NO: 369 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSSGNPPPKK

SEQ ID NO: 370 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSSGEPPPKK

SEQ ID NO: 371 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSSGQPPPKK

SEQ ID NO: 372 - protein, 40 aa, synthetic construct; identical to SEQ ID NO: 103
  REGION 1..40: note = Component of peptide hormone analogue
  MOD_RES 2: note = Aib
  MOD_RES 40: note = The residue at this position is Lys modified with a Y group attached to its epsilon amino group

SEQ ID NO: 373 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKAGGPSSGNPPPGKK

SEQ ID NO: 374 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKAGGPSSGQPPPGKK

SEQ ID NO: 375 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSSGNPPPGK

SEQ ID NO: 376 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKAGGPSSGKPPPGK

SEQ ID NO: 377 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVREFIEWLKAGGPSSGNPPPGK

SEQ ID NO: 378 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVREFIEWLKAGGPSSGEPPPGK

SEQ ID NO: 379 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVREFIEWLKAGGPSSGQPPPGK

SEQ ID NO: 380 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVREFIEWLKAGGPSSGHPPPGK

SEQ ID NO: 381 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVREFIEWLKAGGPSSGKPPPGK

SEQ ID NO: 382 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKAGGPSSGNPPPGK

SEQ ID NO: 383 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKAGGPSSGEPPPGK

SEQ ID NO: 384 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKAGGPSSGQPPPGK

SEQ ID NO: 385 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKAGGPSSGHPPPGK

SEQ ID NO: 386 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVHEFIEWLKAGGPSSGKPPPGK

SEQ ID NO: 387 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVREFIEWLKHGGPSSGKPPPKK

SEQ ID NO: 388 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVREFIEWLKAGGPSSGKPPPKK

SEQ ID NO: 389 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKAVKEFIEWLKAGGPSSGKPPPKK

SEQ ID NO: 390 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKRVREFIEWLKHGGPSSGKPPPGKK

SEQ ID NO: 391 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKHGGPSSGKPPPGKK

SEQ ID NO: 392 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVKEFIEWLKAGGPSSGNPPPGKK

SEQ ID NO: 393 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVKEFIEWLKAGGPSSGNPPPGKK

SEQ ID NO: 394 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVREFIEWLKAGGPSSGQPPPGKK

SEQ ID NO: 395 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVKEFIEWLKAGGPSSGNPPPGKK

SEQ ID NO: 396 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVKEFIEWLKAGGPSSGQPPPGKK

SEQ ID NO: 397 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVKEFIEWLKAGGPSSGHPPPGKK

SEQ ID NO: 398 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKQGGPSSGKPPPGKK

SEQ ID NO: 399 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVKEFIEWLKQGGPSSGKPPPGKK

SEQ ID NO: 400 - protein, 41 aa, synthetic construct; identical to SEQ ID NO: 251
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 401 - protein, 41 aa, synthetic construct; identical to SEQ ID NO: 261
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 402 - protein, 41 aa, synthetic construct; identical to SEQ ID NO: 251
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 403 - protein, 41 aa, synthetic construct; identical to SEQ ID NO: 261
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 404 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVHEFIEWLKHGGPSSGKPPPGKK

SEQ ID NO: 405 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKAGGPSSGQPPPKK

SEQ ID NO: 406 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKAGGPSSGNPPPKK

SEQ ID NO: 407 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKAGGPSSGEPPPKK

SEQ ID NO: 408 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVRLFIEWLKAGGPSHGKPPPKK

SEQ ID NO: 409 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVRLFIEWLKNAGPSSGKPPPGKK

SEQ ID NO: 410 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEERVREFIEWLKNAGPSSGKPPPGKK

SEQ ID NO: 411 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEERVREFIEWLKNAGPSSGKPPPKK

SEQ ID NO: 412 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVKEFIEWLKAGGPSSGHPPPGK

SEQ ID NO: 413 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDLSKQLEEKRVREFIEWLKAGGPSSGHPPPGK

SEQ ID NO: 414 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKNGGPSSGHPPPGK

SEQ ID NO: 415 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKNAGPSSGHPPPGK

SEQ ID NO: 416 - protein, 40 aa, synthetic construct
  REGION 1..40: note = Component of peptide hormone analogue
  MOD_RES 2: note = Aib
  MOD_RES 40: note = The residue at this position is Lys modified with a Y group attached to its epsilon amino group
HXHGTFTSDYSKQLEEKRVREFIEWLKHGGPSSGHPPPGK

SEQ ID NO: 417 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKRVREFIEWLKAGGPSSGHQPPPGKK

SEQ ID NO: 418 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKRVREFIEWLKAGGPSSGQHPPPGKK

SEQ ID NO: 419 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKRVREFIEWLKAGGPSSGKQPPPGKK

SEQ ID NO: 420 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKRVREFIEWLKAGGPSSGQQPPPGKK

SEQ ID NO: 421 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKRVREFIEWLKAGGPSSGEFPPPGKK

SEQ ID NO: 422 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKRVREFIEWLKAGGPSSGKFPPPGKK

SEQ ID NO: 423 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKAGGPSSGKFPPGKK

SEQ ID NO: 424 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKAGGPSSGKQPPGKK

SEQ ID NO: 425 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKRVREFIEWLKAGGPSSGPSPPPGKK

SEQ ID NO: 426 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKRVREFIEWLKAGGPSSGKSPPPGKK

SEQ ID NO: 427 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVQEFIEWLKAGGPSSGKPPPGKK

SEQ ID NO: 428 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKAVHEFIEWLKAGGPSSGKPPPGKK

SEQ ID NO: 429 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKAVREFIEWLKAGGPSSGKPPPGKK

SEQ ID NO: 430 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEERVREFIEWLKAGGPSSGKPPPGKK

SEQ ID NO: 431 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKAVHEFIEWLKAGGPSSGKPPPGKK

SEQ ID NO: 432 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKAVREFIEWLKAGGPSSGKPPPGKK

SEQ ID NO: 433 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEERVREFIEWLKAGGPSSGKPPPGKK

SEQ ID NO: 434 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHEFIEWLKAGGPSSGQPPPGKK

SEQ ID NO: 435 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKAGGPSSGQPPPGKK

SEQ ID NO: 436 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKAGGPSSGKKPPGKK

SEQ ID NO: 437 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKAGGPSSGKRPPGKK

SEQ ID NO: 438 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKAVREFIEWLKAGGPSSGKKPPPGKK

SEQ ID NO: 439 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKAVREFIEWLKAGGPSSGKRPPPGKK

SEQ ID NO: 440 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKAGGPSSGRPPPGKK

SEQ ID NO: 441 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDYSKQLEEKAVREFIEWLKAGGPSSGKEPPPGKK

SEQ ID NO: 442 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKAGGPSSGNPPPGKK

SEQ ID NO: 443 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKAGGPSSGEPPPGKK

SEQ ID NO: 444 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKNAGPSSGKPPPGKK

SEQ ID NO: 445 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKHGGPSSGKPPPGKK

SEQ ID NO: 446 - protein, 41 aa, synthetic construct; identical to SEQ ID NO: 398
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 447 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKQGGPSSGNPPPGKK

SEQ ID NO: 448 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKQGGPSSGEPPPGKK

SEQ ID NO: 449 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKQGGPSSGQPPPGKK

SEQ ID NO: 450 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKQGGPSSGHPPPGKK

SEQ ID NO: 451 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKRVREFIEWLKQGGPSSGKPPPGKK

SEQ ID NO: 452 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKAVREFIEWLKQGGPSSGKPPPGKK

SEQ ID NO: 453 - protein, 41 aa, synthetic construct; identical to SEQ ID NO: 451
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 454 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKAVREFIEWLKQGGPSSGKPPPGKK

SEQ ID NO: 455 - protein, 41 aa, synthetic construct; identical to SEQ ID NO: 301
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 456 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKAVREFIEWLKAGGPSSGNPPPGKK

SEQ ID NO: 457 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKAVREFIEWLKQGGPSSGNPPPGKK

SEQ ID NO: 458 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKRVREFIEWLKAGGPSSGNPPPGKK

SEQ ID NO: 459 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKRVREFIEWLKQGGPSSGNPPPGKK

SEQ ID NO: 460 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKRVREFIEWLKAGGPSSGKPPPGKK

SEQ ID NO: 461 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKRVREFIEWLKQGGPSSGKPPPGKK

SEQ ID NO: 462 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKRVREFIEWLKHGGPSSGNPPPGKK

SEQ ID NO: 463 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKRVREFIEWLKAGGPSSGNPPPGKK

SEQ ID NO: 464 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKRVREFIEWLKQGGPSSGNPPPGKK

SEQ ID NO: 465 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKAVREFIEWLKQGGPSSGQPPPGKK

SEQ ID NO: 466 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKRVREFIEWLKQGGPSSGQPPPGKK

SEQ ID NO: 467 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKAVREFIEWLKAGGPSSGEPPPGKK

SEQ ID NO: 468 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKAVREFIEWLKAGGPSSGQPPPGKK

SEQ ID NO: 469 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKAVREFIEWLKAGGPSSGHPPPGKK

SEQ ID NO: 470 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKRVREFIEWLKQGGPSSGQPPPGKK

SEQ ID NO: 471 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKAVREFIEWLKAGGPSSGQPPPGKK

SEQ ID NO: 472 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKRVREFIEWLKAGGPSSGKPPPGKK

SEQ ID NO: 473 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKAVHEFIEWLKQGGPSSGKPPPGKK

SEQ ID NO: 474 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKAVHEFIEWLKQGGPSSGHPPPGKK

SEQ ID NO: 475 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKAVHEFIEWLKQGGPSSGQPPPGKK

SEQ ID NO: 476 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKAVHEFIEWLKAGGPSSGHPPPGKK

SEQ ID NO: 477 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKAVHEFIEWLKAGGPSSGQPPPGKK

SEQ ID NO: 478 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKAVREFIEWLKQGGPSSGQPPPGKK

SEQ ID NO: 479 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVHEFIEWLKQGGPSSGKPPPGKK

SEQ ID NO: 480 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDLSKQLEEKAVHEFIEWLKQGGPSSGKPPPGKK

SEQ ID NO: 481 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKRVREFIEWLKAGGPSSGQPPPGKK

SEQ ID NO: 482 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKRVREFIEWLKQGGPSSGQPPPGKK

SEQ ID NO: 483 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDYSKQLEEKAVREFIEWLKQGGPSSGKKPPGKK

SEQ ID NO: 484 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKAVREFIEWLKQGGPSSGKKPPGKK

SEQ ID NO: 485 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKAVREFIEWLKAGGPSSGKKPPGKK

SEQ ID NO: 486 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXHGTFTSDVSKQLEEKAVREFIEWLKAGGPSSGKKPPPGKK

SEQ ID NO: 487 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKRVREFIEWLKQGGPSSGKKPPGKK

SEQ ID NO: 488 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXHGTFTSDVSKQLEEKRVREFIEWLKAGGPSSGKKPPGKK

SEQ ID NO: 489 - protein, 41 aa, synthetic construct; identical to SEQ ID NO: 454
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 490 - protein, 41 aa, synthetic construct; identical to SEQ ID NO: 299
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 491 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
YXHGTFTSDVSKQLEEKAVREFIEWLKQGGPSSGKPPPGKK

SEQ ID NO: 492 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXHGTFTSDVSKQLEEKAVREFIEWLKQGGPSSGKPPPGKK

SEQ ID NO: 493 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
YXEGTFTSDVSKQLEEKAVREFIEWLKQGGPSSGKPPPGKK

SEQ ID NO: 494 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
YXHGTFTSDYSKQLEEKAVREFIEWLKQGGPSSGKPPPGKK

SEQ ID NO: 495 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXHGTFTSDYSKQLEEKAVREFIEWLKQGGPSSGKPPPGKK

SEQ ID NO: 496 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
YXEGTFTSDYSKQLEEKAVREFIEWLKQGGPSSGKPPPGKK

SEQ ID NO: 497 - protein, 41 aa, synthetic construct; identical to SEQ ID NO: 311
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 498 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
YXHGTFTSDYSKQLEEKRVREFIEWLKQGGPSSGKPPPGKK

SEQ ID NO: 499 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXHGTFTSDYSKQLEEKRVREFIEWLKQGGPSSGKPPPGKK

SEQ ID NO: 500 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
YXEGTFTSDYSKQLEEKRVREFIEWLKQGGPSSGKPPPGKK

SEQ ID NO: 501 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXEGTFTSDYSKQLEEKRVREFIEWLKQGGPSSGKPPPGKK

SEQ ID NO: 502 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXEGTFTSDVSKQLEEKRVREFIEWLKQGGPSSGKPPPGKK

SEQ ID NO: 503 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXEGTFTSDLSKQLEEKRVREFIEWLKQGGPSSGKPPPGKK

SEQ ID NO: 504 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXEGTFTSDYSKQLEEKAVREFIEWLKQGGPSSGKPPPGKK

SEQ ID NO: 505 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXEGTFTSDVSKQLEEKAVREFIEWLKQGGPSSGKPPPGKK

SEQ ID NO: 506 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXEGTFTSDLSKQLEEKAVREFIEWLKQGGPSSGKPPPGKK

SEQ ID NO: 507 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXEGTFTSDYSKQLEEKRVREFIEWLKQGGPSSGKPPPGKK

SEQ ID NO: 508 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXEGTFTSDYSKQLEEKAVREFIEWLKAGGPSSGKPPPGKK

SEQ ID NO: 509 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXEGTFTSDVSKQLEEKAVREFIEWLKQGGPSSGKPPPGKK

SEQ ID NO: 510 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXEGTFTSDYSKQLEEKAVREFIEWLKQGGPSSGKPPPGKK

SEQ ID NO: 511 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXEGTFTSDLSKQLEEKAVREFIEWLKQGGPSSGKPPPGKK

SEQ ID NO: 512 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXQGTFTSDVSKQLEEKAVREFIEWLKQGGPSSGKPPPGKK

SEQ ID NO: 513 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXQGTFTSDVSKQLEEKRVREFIEWLKQGGPSSGKPPPGKK

SEQ ID NO: 514 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXQGTFTSDVSKQLEEKRVREFIEWLKQGGPSSGKPPPGKK

SEQ ID NO: 515 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXEGTFTSDVSKQLEEKRVREFIEWLKQGGPSSGKPPPGKK

SEQ ID NO: 516 - protein, 41 aa, synthetic construct; identical to SEQ ID NO: 493
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 517 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
YXEGTFTSDVSKQLEEKRVREFIEWLKQGGPSSGKPPPGKK

SEQ ID NO: 518 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
YXQGTFTSDVSKQLEEKAVREFIEWLKQGGPSSGKPPPGKK

SEQ ID NO: 519 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
HXQGTFTSDVSKQLEEKAVREFIEWLKQGGPSSGKPPPGKK

SEQ ID NO: 520 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXQGTFTSDVSKQLEEKAVREFIEWLKAGGPSSGKKPPPGKK

SEQ ID NO: 521 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXQGTFTSDVSKQLEEKAVREFIEWLKQGGPSSGKKPPPGKK

SEQ ID NO: 522 - protein, 42 aa, synthetic construct
  REGION 1..42: note = Component of peptide hormone analogue
  MOD_RES 2: note = Aib
  MOD_RES 42: note = The residue at this position is Lys modified with a Y group attached to its epsilon amino group
HXQGTFTSDVSKQLEEKRVREFIEWLKQGGPSSGKKPPPGKK

SEQ ID NO: 523 - protein, 41 aa, synthetic construct; identical to SEQ ID NO: 502
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 524 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXEGTFTSDVSKQLEEKRVREFIEWLKAGGPSSGKPPPGKK

SEQ ID NO: 525 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXEGTFTSDVSKQLEEKRVREFIEWLKQGGPSSGEPPPGKK

SEQ ID NO: 526 - protein, 41 aa, synthetic construct
  REGION 1..41: note = Component of peptide hormone analogue
  MOD_RES 2: note = Aib
  MOD_RES 41: note = The residue at this position is Lys modified with a Y group attached to its epsilon amino group
FXEGTFTSDVSKQLEEKRVREFIEWLKQGGPSSGQPPPGKK

SEQ ID NO: 527 - protein, 41 aa, synthetic construct; identical to SEQ ID NO: 502
  REGION 1..41: note = Component of peptide hormone analogue
  MOD_RES 2: note = Aib
  MOD_RES 41: note = The residue at this position is Lys modified with a Y group attached to its epsilon amino group

SEQ ID NO: 528 - protein, 42 aa, synthetic construct
  REGION 1..42: note = Component of peptide hormone analogue
  VARIANT 1: note = The residue at this position is His, Tyr or Phe
  MOD_RES 2: note = Aib
  VARIANT 3: note = The residue at this position is His, Gln or Glu
  VARIANT 10: note = The residue at this position is Leu, Tyr or Val
  VARIANT 17: note = The residue at this position is Glu or Lys
  VARIANT 18: note = The residue at this position is Ala or Arg
  VARIANT 20: note = The residue at this position is Arg, Gln, Lys or His
  VARIANT 21: note = The residue at this position is Leu, Ala, Arg, Lys or Glu
  VARIANT 28: note = The residue at this position is Ala, Asn, Gln, His
  VARIANT 29: note = The residue at this position is Gly or Ala
  VARIANT 33: note = The residue at this position is Ser, His or Gln
  VARIANT 35: note = The residue at this position is Lys, Pro, His, Arg, Asn, Gln, Glu, Ser or Ala
  VARIANT 36: note = The residue at this position is Phe, His, Gln, Glu, Lys, Ser, Arg or absent
  VARIANT 39: note = The residue at this position is Pro or Gly
  VARIANT 40: note = The residue at this position is Gly or absent
  VARIANT 41: note = The residue at this position is Lys or absent
  MOD_RES 42: note = The residue at this position is Lys modified with a Y2 group attached to its epsilon amino group
XXXGTFTSDXSKQLEEXXVXXFIEWLKXXGPSXGXXPPXXXK

SEQ ID NO: 529 - protein, 31 aa, Homo sapiens
HAEGTFTSDVSSYLEGQAAKEFIAWLVKGRG

SEQ ID NO: 530 - protein, 30 aa, Homo sapiens
  MOD_RES 30: note = AMIDATION
HAEGTFTSDVSSYLEGQAAKEFIAWLVKGR

SEQ ID NO: 531 - protein, 29 aa, Homo sapiens
HSQGTFTSDYSKYLDSRRAQDFVQWLMNT

SEQ ID NO: 532 - protein, 37 aa, Homo sapiens
HSQGTFTSDYSKYLDSRRAQDFVQWLMNTKRNRNNIA

SEQ ID NO: 533 - protein, 5 aa, synthetic construct
  REGION 1..5: note = Component of peptide hormone analogue
PPPGK

SEQ ID NO: 534 - protein, 4 aa, synthetic construct
  REGION 1..4: note = Component of peptide hormone analogue
PPPK

SEQ ID NO: 535 - protein, 5 aa, synthetic construct
  REGION 1..5: note = Component of peptide hormone analogue
DDDDY

SEQ ID NO: 536 - protein, 5 aa, synthetic construct
  REGION 1..5: note = Component of peptide hormone analogue
HPFHL